SEQUENCE LISTING (110 sequences)

SEQ ID NO: 1 - DNA, 514 bp, Artificial Sequence
  source: /note=“Description of Artificial Sequence Synthetic polynucleotide”
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccgtgataaaaggcgtacataattcttgtgtctactgtacagaatactgccgccagctggatttcccaattctgagtaacactctgcaatccaaacagggttcgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 2 - DNA, 510 bp, Artificial Sequence
  source: /note=“Description of Artificial Sequence Synthetic polynucleotide”
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcagaccgtgataaaaggcgtacataattcttgtgtctactgtacagaatactgccgccagctggatttcccaattctgagtaacactctgcaatccaaacagggttcgtggaatttttggagtaggctttctggctttttaccggaaagcccctcttatgatgtttgttgccaatgatagattgttttcactgtgcaaaaattatgggtagttttggtggtcttgatgcagttgtaagcttggggtatg

SEQ ID NO: 3 - DNA, 698 bp, Artificial Sequence
  source: /note=“Description of Artificial Sequence Synthetic polynucleotide”
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgtgataaaaggcgtacataattcttgtgtctactgtacagaatactgccgccagctggatttcccaattctgagtaacactctgcaatccaaacagggttcgtggcaggggagataccatgatcacgaaggtggttttcccagggcgaggcttatccattgcactccggatgtgctgacccctgcgatttccccaaatgtgggaaactcgactgcataatttgtggtagtgggggactgcgttcgcgctttcccctgactttctggagtttcaaaaacagactgtacgccaagggtcatatc

SEQ ID NO: 4 - DNA, 650 bp, Artificial Sequence
  source: /note=“Description of Artificial Sequence Synthetic polynucleotide”
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgtgataaaaggcgtacataattcttgtgtctactgtacagaatactgccgccagctggatttcccaattctgagtaacactctgcaatccaaacagggttcgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 5 - DNA, 513 bp, Artificial Sequence
  source: /note=“Description of Artificial Sequence Synthetic polynucleotide”
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccgggggctccgggccgggcgcgtcgcgagggctcccgccgaggaggagaccaaacggaggacagaagcgagaaggtccaagttctggttccagggaactctgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 6 - DNA, 510 bp, Artificial Sequence
  source: /note=“Description of Artificial Sequence Synthetic polynucleotide”
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcagaccgtgactagcctgtcatccaccatcacctccttggtcagaaagtcagctcccattgtggctttgtactgattgctgaatttcttattcacatactggttcatgtggaatttttggagtaggctttctggctttttaccggaaagcccctcttatgatgtttgttgccaatgatagattgttttcactgtgcaaaaattatgggtagttttggtggtcttgatgcagttgtaagcttggggtatg

SEQ ID NO: 7 - DNA, 514 bp, Artificial Sequence
  source: /note=“Description of Artificial Sequence Synthetic polynucleotide”
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccggccacaactccctccttggcctttgaaagtcctttcatgaatacatccacggctaatgaattcctttacaccacactgtcgtcgaatggccactcccagtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 8 - DNA, 514 bp, Artificial Sequence
  source: /note=“Description of Artificial Sequence Synthetic polynucleotide”
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaccgaacatcgtagattgaagccacaaaatccacagcacacaaagaccctgccaccatgtattcacttcagtgaaagggaagcaccgaaatgctgagtgggggcgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 9 - DNA, 100 bp, Artificial Sequence
  source: /note=“Description of Artificial Sequence Synthetic polynucleotide”
tgataaaaggcgtacataattcttgtgtctactgtacagaatactgccgccagctggatttcccaattctgagtaacactctgcaatccaaacagggttc

SEQ ID NO: 10 - DNA, 100 bp, Artificial Sequence
  source: /note=“Description of Artificial Sequence Synthetic polynucleotide”
gggggctccgggccgggcgcgtcgcgagggctcccgccgaggaggagaccaaacggaggacagaagcgagaaggtccaagttctggttccagggaactct

SEQ ID NO: 11 - DNA, 100 bp, Artificial Sequence
  source: /note=“Description of Artificial Sequence Synthetic polynucleotide”
tgactagcctgtcatccaccatcacctccttggtcagaaagtcagctcccattgtggctttgtactgattgctgaatttcttattcacatactggttcat

SEQ ID NO: 12 - DNA, 99 bp, Artificial Sequence
  source: /note=“Description of Artificial Sequence Synthetic oligonucleotide”
tttcaggatctcggggactggcctggatgagaaactcatctctccagccatctagggtttggaatgtgttgggggcagtcacatcaaataccagaacgc

SEQ ID NO: 13 - DNA, 100 bp, Artificial Sequence
  source: /note=“Description of Artificial Sequence Synthetic polynucleotide”
aagtactcacgcagaatctactggccagaagttgatcagagtaacgggaccgcaaaacaaaaaatgaggtggtgaaggagacacacgcaaactcagccgc

SEQ ID NO: 14 - DNA, 100 bp, Artificial Sequence
  source: /note=“Description of Artificial Sequence Synthetic polynucleotide”
ctggttcaaactttggcagtaatgctggattaacaaatgttcatcatctccggaaaataaaatcaaaggttgtggtttgttcccccccttatgttgcttt

SEQ ID NO: 15 - DNA, 100 bp, Artificial Sequence
  source: /note=“Description of Artificial Sequence Synthetic polynucleotide”
gccacaactccctccttggcctttgaaagtcctttcatgaatacatccacggctaatgaattcctttacaccacactgtcgtcgaatggccactcccagt

SEQ ID NO: 16 - DNA, 100 bp, Artificial Sequence
  source: /note=“Description of Artificial Sequence Synthetic polynucleotide”
aacatcgtagattgaagccacaaaatccacagcacacaaagaccctgccaccatgtattcacttcagtgaaagggaagcaccgaaatgctgagtgggggc

SEQ ID NO: 17 - DNA, 100 bp, Artificial Sequence
  source: /note=“Description of Artificial Sequence Synthetic polynucleotide”
agccagtggctgctgcaatgctccctgctccctccactgtcttctgggccactgctgtcacacccgtcaccactgctcctccaacatttgtcacttgctc

SEQ ID NO: 18 - DNA, 146 bp, Artificial Sequence
  source: /note=“Description of Artificial Sequence Synthetic polynucleotide”
gtgccctctgatgtttttatccccattctacagcattacggagcagtgccgtagtgtcgtttctttgcaatgatggcagcattgggatacagtgtgaaaagtggaatttttggagcaggttttctgacttcggtcggaaaacccct

SEQ ID NO: 19 - DNA, 146 bp, Artificial Sequence
  source: /note=“Description of Artificial Sequence Synthetic polynucleotide”
ctggcaacttcaggtgcacgaaaccctggtgtgccctctgatgtttttatccccattctacagcattacggagcagtgccgtagtgtcgtttctttgcaagtggaatttttggagcaggttttctgacttcggtcggaaaacccct

SEQ ID NO: 20 - DNA, 100 bp, Artificial Sequence
  source: /note=“Description of Artificial Sequence Synthetic polynucleotide”
gccggcaccattcactcccaggaggccaaagcactctccagtgagaactcggaccacagcctcccgctgctgggctggaggtgcctggataaatcttggt

SEQ ID NO: 21 - DNA, 100 bp, Artificial Sequence
  source: /note=“Description of Artificial Sequence Synthetic polynucleotide”
ccccagtgagcatcttgaatgtggttgtattgccggcaccattcactcccaggaggccaaagcactctccagtgagaactcggaccacagcctcccgctg

SEQ ID NO: 22 - DNA, 146 bp, Artificial Sequence
  source: /note=“Description of Artificial Sequence Synthetic polynucleotide”
ccccagtgagcatcttgaatgtggttgtattgccggcaccattcactcccaggaggccaaagcactctccagtgagaactcggaccacagcctcccgctggtggaatttttggagcaggttttctgacttcggtcggaaaacccct

SEQ ID NO: 23 - DNA, 146 bp, Artificial Sequence
  source: /note=“Description of Artificial Sequence Synthetic polynucleotide”
cccagtgagcatcttgaatgtggttgttttgccggcaccattcactcccaggaggccaaagcacgctccagggcgaacttatcacatacagcctgtccacgtggaatttttggagcaggttttctgacttcggtcggaaaacccct

SEQ ID NO: 24 - DNA, 100 bp, Artificial Sequence
  source: /note=“Description of Artificial Sequence Synthetic polynucleotide”
gccctgcactgggccgtcgcccttcagcacgcacacggccttcgtcgccacaactcgctaggccacgccgaggtcctggttccgaggactgcaacggaaa

SEQ ID NO: 25 - DNA, 120 bp, Artificial Sequence
  source: /note=“Description of Artificial Sequence Synthetic polynucleotide”
tatgatagggacttagggtggccctgcactgggccgtcgcccttcagcacgcacacggccttcgtcgccacaactcgctaggccacgccgaggtcctggttccgaggactgcaacggaaa

SEQ ID NO: 26 - RNA, 150 nt, Artificial Sequence
  source: /note=“Description of Artificial Sequence Synthetic polynucleotide”
accgauggguauggccucuaaaaacauggccccagcagcuucagucccuuucucgucgauggucagcacagccuuaugcacggccuggaggggagagaagcagaguggaauuuuuggagcagguuuucugacuucggucggaaaaccccu

SEQ ID NO: 27 - RNA, 150 nt, Artificial Sequence
  source: /note=“Description of Artificial Sequence Synthetic polynucleotide”
accgauggguauggccucuaaaaacauggccccagcagcuucagucccuuacucgucgauggucagcacagccuuaugcacggccuggaggggagagaagcagaguggaauuuuuggagcagguuuucugacuucggucggaaaaccccu

SEQ ID NO: 28 - DNA, 572 bp, Artificial Sequence
  source: /note=“Description of Artificial Sequence Synthetic polynucleotide”
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgagagaccagctggatggtctcagtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 29 - DNA, 670 bp, Artificial Sequence
  source: /note=“Description of Artificial Sequence Synthetic polynucleotide”
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgtatgatagggacttagggtgtgataaaaggcgtacataattcttgtgtctactgtacagaatactgccgccagctggatttcccaattctgagtaacactctgcaatccaaacagggttcgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 30 - DNA, 770 bp, Artificial Sequence
  source: /note=“Description of Artificial Sequence Synthetic polynucleotide”
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctcaccgtatgatagggacttagggtgtcttgtgtctactgtacagaatactgccgccagctggatttcccaattctgagtaacactctgcaatccaaacagggttcaaccctccaccttacaggcctgcattacaggacttaaacacataatccaagaatttcttacactaatttatacattttaattggttgcatatattaacatgtactataagattcttttctgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 31 - DNA, 163 bp, Artificial Sequence
  source: /note=“Description of Artificial Sequence Synthetic polynucleotide”
taatctttgaaagtcctttcatgaatacatccacggctaatgaattcctttacaccacattagccagaaggcttgaaggcaaggcgtgagggagcgcccaggacgctctcggagatatataaatttttggagcaggttttctgacttcggtcggaaaacccct

SEQ ID NO: 32 - DNA, 188 bp, Artificial Sequence
  source: /note=“Description of Artificial Sequence Synthetic polynucleotide”
taatctttgaaagtcctttcatgaatacatccacggctaatgaattcctttacaccacaaggggcgaatggccactcccagttctccgctcacgagggtggaaataattaaggcgtgagggagcgcccaggacgctctcatatataaatttttggagcaggttttctgacttcggtcggaaaacccct

SEQ ID NO: 33 - DNA, 166 bp, Artificial Sequence
  source: /note=“Description of Artificial Sequence Synthetic polynucleotide”
taattttctcagcagcagccacaactccgaggaacccctttgaaagtcctttcatgaatacatccacggctaaacttctcctttacaccacactgtcgtcgaatggccactcccagtatatataaatttttggagcaggttttctgacttcggtcggaaaacccct

SEQ ID NO: 34 - DNA, 249 bp, Artificial Sequence
  source: /note=“Description of Artificial Sequence Synthetic polynucleotide”
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 35 - DNA, 341 bp, Artificial Sequence
  source: /note=“Description of Artificial Sequence Synthetic polynucleotide”
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 36 - DNA, 263 bp, Artificial Sequence
  source: /note=“Description of Artificial Sequence Synthetic polynucleotide”
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 37 - DNA, 264 bp, Artificial Sequence
  source: /note=“Description of Artificial Sequence Synthetic polynucleotide”
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgcag

SEQ ID NO: 38 - DNA, 257 bp, Artificial Sequence
  source: /note=“Description of Artificial Sequence Synthetic polynucleotide”
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 39 - DNA, 393 bp, Artificial Sequence
  source: /note=“Description of Artificial Sequence Synthetic polynucleotide”
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 40 - DNA, 698 bp, Artificial Sequence
  source: /note=“Description of Artificial Sequence Synthetic polynucleotide”
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacc

SEQ ID NO: 41 - DNA, 11 bp, Artificial Sequence
  source: /note=“Description of Artificial Sequence Synthetic oligonucleotide”
aatttttggag

SEQ ID NO: 42 - DNA, 31 bp, Artificial Sequence
  source: /note=“Description of Artificial Sequence Synthetic oligonucleotide”
caggttttctgacttcggtcggaaaacccct

SEQ ID NO: 43 - DNA, 32 bp, Artificial Sequence
  source: /note=“Description of Artificial Sequence Synthetic oligonucleotide”
taggctttctggctttttaccggaaagcccct

SEQ ID NO: 44 - DNA, 42 bp, Artificial Sequence
  source: /note=“Description of Artificial Sequence Synthetic oligonucleotide”
aatttktggagcaggytttctgacttcggtcggaaarcccct

SEQ ID NO: 45 - DNA, 149 bp, Artificial Sequence
  source: /note=“Description of Artificial Sequence Synthetic polynucleotide”
aatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 46 - DNA, 154 bp, Artificial Sequence
  source: /note=“Description of Artificial Sequence Synthetic polynucleotide”
ggcaggggagataccatgatcacgaaggtggttytcccagggcgaggcttatccattgcactccggatgtgctgacccctgcgatttccccaaatgtgggaaactcgactgcataatttgtggtagtgggggactgcgttcgcgctttcccctg

SEQ ID NO: 47 - DNA, 20 bp, Artificial Sequence
  source: /note=“Description of Artificial Sequence Synthetic oligonucleotide”
tatgatagggacttagggtg

SEQ ID NO: 48 - DNA, 18 bp, Artificial Sequence
  source: /note=“Description of Artificial Sequence Synthetic oligonucleotide”
tagggatagggataggga

SEQ ID NO: 49 - RNA, 45 nt, Artificial Sequence
  source: /note=“Description of Artificial Sequence Synthetic oligonucleotide”
guggaauaguauaacaauaugcuaaauguuguuauaguaucccac

SEQ ID NO: 50 - DNA, 5 bp, Artificial Sequence
  source: /note=“Description of Artificial Sequence Synthetic oligonucleotide”
atata

SEQ ID NO: 51 - DNA, 5 bp, Artificial Sequence
  source: /note=“Description of Artificial Sequence Synthetic oligonucleotide”
ataat

SEQ ID NO: 52 - RNA, 5 nt, Artificial Sequence
  source: /note=“Description of Artificial Sequence Synthetic oligonucleotide”
auaau

SEQ ID NO: 53 - RNA, 5 nt, Artificial Sequence
  source: /note=“Description of Artificial Sequence Synthetic oligonucleotide”
auaua

SEQ ID NO: 54 - RNA, 5 nt, Artificial Sequence
  source: /note=“Description of Artificial Sequence Synthetic oligonucleotide”
uaaua

SEQ ID NO: 55 - DNA, 11 bp, Artificial Sequence
  source: /note=“Description of Artificial Sequence Synthetic oligonucleotide”
agtttttttta

SEQ ID NO: 56 - DNA, 12 bp, Artificial Sequence
  source: /note=“Description of Artificial Sequence Synthetic oligonucleotide”
agaaaaaaaata

SEQ ID NO: 57 - RNA, 11 nt, Artificial Sequence
  source: /note=“Description of Artificial Sequence Synthetic oligonucleotide”
aauuuuuggag

SEQ ID NO: 58 - RNA, 11 nt, Artificial Sequence
  source: /note=“Description of Artificial Sequence Synthetic oligonucleotide”
aauuugucuag

SEQ ID NO: 59 - protein, 9 aa, Artificial Sequence
  source: /note=“Description of Artificial Sequence Synthetic peptide”
VGGAVVTGV

SEQ ID NO: 60 - protein, 6 aa, Artificial Sequence
  source: /note=“Description of Artificial Sequence Synthetic peptide”
DPDNEA

SEQ ID NO: 61 - DNA, 54 bp, Homo sapiens
tgtctactgtacagaatactgccgctagctggatttcccaattctgagtaacac

SEQ ID NO: 62 - DNA, 54 bp, Artificial Sequence; identical to SEQ ID NO: 61
  source: /note=“Description of Artificial Sequence Synthetic oligonucleotide”

SEQ ID NO: 63 - DNA, 54 bp, Artificial Sequence
  source: /note=“Description of Artificial Sequence Synthetic oligonucleotide”
tgtccactgtacagaatactgccgccagctggatttcccaattctgagtaacac

SEQ ID NO: 64 - DNA, 11 bp, Homo sapiens
cgtttagtctc

SEQ ID NO: 65 - DNA, 11 bp, Artificial Sequence
  source: /note=“Description of Artificial Sequence Synthetic oligonucleotide”
cgtttagtctc

SEQ ID NO: 66 - DNA, 11 bp, Artificial Sequence
  source: /note=“Description of Artificial Sequence Synthetic oligonucleotide”
cgtttggtctc

SEQ ID NO: 67 - DNA, 11 bp, Homo sapiens
acaataggagc

SEQ ID NO: 68 - DNA, 11 bp, Artificial Sequence
  source: /note=“Description of Artificial Sequence Synthetic oligonucleotide”
acaataggagc

SEQ ID NO: 69 - DNA, 14 bp, Homo sapiens
ccagctagcggcag

SEQ ID NO: 70 - DNA, 14 bp, Artificial Sequence
  source: /note=“Description of Artificial Sequence Synthetic oligonucleotide”
ccagctagcggcag

SEQ ID NO: 71 - DNA, 16 bp, Homo sapiens
attagccatggatgta

SEQ ID NO: 72 - DNA, 16 bp, Artificial Sequence
  source: /note=“Description of Artificial Sequence Synthetic oligonucleotide”
attagccatggatgta

SEQ ID NO: 73 - DNA, 15 bp, Homo sapiens
tacatggtagcaggg

SEQ ID NO: 74 - DNA, 15 bp, Artificial Sequence
  source: /note=“Description of Artificial Sequence Synthetic oligonucleotide”
tacatggtagcaggg

SEQ ID NO: 75 - DNA, 15 bp, Artificial Sequence
  source: /note=“Description of Artificial Sequence Synthetic oligonucleotide”
tacatggtggcaggg

SEQ ID NO: 76 - protein, 6 aa, Artificial Sequence
  source: /note=“Description of Artificial Sequence Synthetic peptide”
  MOD_RES (2)..(2): Any amino acid
KXKEGV

SEQ ID NO: 77 - DNA, 34 bp, Homo sapiens
tacagaatactgccgctagctggatttcccaatt

SEQ ID NO: 78 - DNA, 34 bp, Artificial Sequence; identical to SEQ ID NO: 77
  source: /note=“Description of Artificial Sequence Synthetic oligonucleotide”

SEQ ID NO: 79 - DNA, 34 bp, Artificial Sequence
  source: /note=“Description of Artificial Sequence Synthetic oligonucleotide”
tacagaatactgccgccagctggatttcccaatt

SEQ ID NO: 80 - DNA, 42 bp, Artificial Sequence
  source: /note=“Description of Artificial Sequence Synthetic oligonucleotide”
aatttttggagcaggttttctgacttcggtcggaaaacccct

SEQ ID NO: 81 - DNA, 42 bp, Artificial Sequence
  source: /note=“Description of Artificial Sequence Synthetic oligonucleotide”
aatttgtggagcaggttttctgacttcggtcggaaaacccct

SEQ ID NO: 82 - DNA, 42 bp, Artificial Sequence
  source: /note=“Description of Artificial Sequence Synthetic oligonucleotide”
aatttgtctagcaggttttctgacttcggtcggaaaacccct

SEQ ID NO: 83 - DNA, 42 bp, Artificial Sequence
  source: /note=“Description of Artificial Sequence Synthetic oligonucleotide”
aacgcgtcatgcaggttttctgacttcggtcggaaaacccct

SEQ ID NO: 84 - DNA, 42 bp, Artificial Sequence
  source: /note=“Description of Artificial Sequence Synthetic oligonucleotide”
aatttttggagcaggctttctgacttcggtcggaaagcccct

SEQ ID NO: 85 - DNA, 43 bp, Artificial Sequence
  source: /note=“Description of Artificial Sequence Synthetic oligonucleotide”
aatttttggagtaggctttctggctttttaccggaaagcccct

SEQ ID NO: 86 - DNA, 17 bp, Homo sapiens
ccctagatagctggaga

SEQ ID NO: 87 - DNA, 17 bp, Artificial Sequence
  source: /note=“Description of Artificial Sequence Synthetic oligonucleotide”
ccctagatagctggaga

SEQ ID NO: 88 - DNA, 18 bp, Homo sapiens
cagcagtagcccagaaga

SEQ ID NO: 89 - DNA, 18 bp, Artificial Sequence
  source: /note=“Description of Artificial Sequence Synthetic oligonucleotide”
cagcagtagcccagaaga

SEQ ID NO: 90 - DNA, 18 bp, Homo sapiens
catggtagcagggtcttt

SEQ ID NO: 91 - DNA, 18 bp, Artificial Sequence
  source: /note=“Description of Artificial Sequence Synthetic oligonucleotide”
catggtggcagggtcttt

SEQ ID NO: 92 - DNA, 18 bp, Artificial Sequence
  source: /note=“Description of Artificial Sequence Synthetic oligonucleotide”
catggtagcagggtcttt

SEQ ID NO: 93 - DNA, 157 bp, Artificial Sequence
  source: /note=“Description of Artificial Sequence Synthetic polynucleotide”
tttcccaattctgagtaacactctgcaatccaaacagggttcgtggatatactgccatcagtcggcgtggactgtagaaccatgccgactgatggcagataataccgtgataaaaggcgtacataattcttgtgtctactgtacagaatactgccgc

SEQ ID NO: 94 - DNA, 129 bp, Artificial Sequence
  source: /note=“Description of Artificial Sequence Synthetic polynucleotide”
  modified_base (1)..(9): a, c, t, g, unknown or other
nnnnnnnnnggttcgtggatatactgccatcagtcggcgtggactgtagaaccatgccgactgatggcagataataccgtgataaaaggcgtacataattcttgtgtctactgtacagaatactgccgc

SEQ ID NO: 95 - DNA, 199 bp, Artificial Sequence
  source: /note=“Description of Artificial Sequence Synthetic polynucleotide”
tttcccaattctgagtaacactctgcaatccaaacagggttcgtggaatttttggagcaggttttctgacttcggtcggaaaacccctatatactgccatcagtcggcgtggactgtagaaccatgccgactgatggcagataataccgtgataaaaggcgtacataattcttgtgtctactgtacagaatactgccgc

SEQ ID NO: 96 - DNA, 171 bp, Artificial Sequence
  source: /note=“Description of Artificial Sequence Synthetic polynucleotide”
  modified_base (1)..(8): a, c, t, g, unknown or other
nnnnnnnncgggttcgtggatttttggagcaggttttctgacttcggtcggaaaacccctatatactgccatcagtcggcgtggactgtagaaccatgccgactgatggcagataataccgtgataaaaggcgtacataattcttgtgtctactgtacagaatactgccga

SEQ ID NO: 97 - DNA, 57 bp, Artificial Sequence
  source: /note=“Description of Artificial Sequence Synthetic oligonucleotide”
aatttttggagcaggttttctgacttcggtcggaaaacccctatatactgccatcag

SEQ ID NO: 98 - DNA, 63 bp, Artificial Sequence
  source: /note=“Description of Artificial Sequence Synthetic oligonucleotide”
aatttttggagcaggttttctgacttcggtcggaaaacccctagttttttttactgccatcag

SEQ ID NO: 99 - DNA, 73 bp, Artificial Sequence
  source: /note=“Description of Artificial Sequence Synthetic oligonucleotide”
aatttttggagcaggttttctgacttcggtcggaaaacccctagacactctttccctacacgactgccatcag

SEQ ID NO: 100 - DNA, 15 bp, Artificial Sequence
  source: /note=“Description of Artificial Sequence Synthetic oligonucleotide”
atatactgccatcag

SEQ ID NO: 101 - DNA, 57 bp, Artificial Sequence
  source: /note=“Description of Artificial Sequence Synthetic oligonucleotide”
aatttgtggagcaggctttctgacttcggtcggaaagcccctatatactgccatcag

SEQ ID NO: 102 - DNA, 64 bp, Artificial Sequence
  source: /note=“Description of Artificial Sequence Synthetic oligonucleotide”
aatttgtggagcaggctttctgacttcggtcggaaagcccctagaaaaaaaatactgccatcag

SEQ ID NO: 103 - DNA, 73 bp, Artificial Sequence
  source: /note=“Description of Artificial Sequence Synthetic oligonucleotide”
aatttgtggagcaggctttctgacttcggtcggaaagcccctagacactctttccctacacgactgccatcag

SEQ ID NO: 104 - DNA, 32 bp, Homo sapiens
aggcacctccagcccagcagtggacaggctgt

SEQ ID NO: 105 - DNA, 35 bp, Homo sapiens
gccctggagagtgctttggcctcctgggagtgaat

SEQ ID NO: 106 - DNA, 32 bp, Artificial Sequence; identical to SEQ ID NO: 104
  source: /note=“Description of Artificial Sequence Synthetic oligonucleotide”

SEQ ID NO: 107 - DNA, 35 bp, Artificial Sequence; identical to SEQ ID NO: 105
  source: /note=“Description of Artificial Sequence Synthetic oligonucleotide”

SEQ ID NO: 108 - RNA, 18 nt, Artificial Sequence
  source: /note=“Description of Artificial Sequence Synthetic oligonucleotide”
uagggauagggauaggga

SEQ ID NO: 109 - RNA, 20 nt, Artificial Sequence
  source: /note=“Description of Artificial Sequence Synthetic oligonucleotide”
uaugauagggacuuagggug

SEQ ID NO: 110 - RNA, 20 nt, Artificial Sequence
  source: /note=“Description of Artificial Sequence Synthetic oligonucleotide”
uaugauacgcacuuacgcug

(19) United States
(12) Patent Application Publication
(10) Pub. No.: US 2023/0203483 A1
(43) Pub. Date: Jun. 29, 2023

(54) Compositions and Methods Using SNRNA Components

(71) Applicant: Shape Therapeutics Inc., Seattle, WA (US)

(72) Inventor: Susan BYRNE, Cambridge, MA (US)

(21) Appl. No.: 17/920,065

(22) PCT Filed: Apr. 22, 2021
(86) PCT No.: PCT/US2021/028618

§ 371 Date: Oct. 20, 2022

Related U.S. Application Data
(60) Provisional application No. 63/013,774, filed on Apr. 22, 2020.
(60) Provisional application No. 63/030,118, filed on May 26, 2020.
(60) Provisional application No. 63/086,434, filed on Oct. 1, 2020.
(60) Provisional application No. 63/112,486, filed on Nov. 11, 2020.
(60) Provisional application No. 63/119,878, filed on Dec. 1, 2020.
(60) Provisional application No. 63/153,817, filed on Feb. 25, 2021.

(51) Int. Cl. C12N 15/113; C12N 9/78; C12N 15/87

(52) U.S. Cl. CPC C12N 15/113; C12N 9/78; C12Y 305/04; C12N 15/87; C12N 2310/11; C12N 2310/531; C12N 2320/33

(57) ABSTRACT

Disclosed herein are compositions, pharmaceutical compositions, and methods of use comprising an engineered polynucleotide that can be used to hybridize with a target RNA which may contain a nucleotide mismatch. Compositions and methods disclosed herein can be used to edit RNA to ameliorate or treat diseases or conditions in a subject.

### CROSS REFERENCE TO RELATED APPLICATIONS

This application claims priority under 35 U.S.C. § 119 from Provisional Application Ser. No. 63/013,744, filed Apr. 22, 2020, Provisional Application Ser. No. 63/030,118, filed May 26, 2020, Provisional Application Ser. No. 63/086,434, filed Oct. 1, 2020, Provisional Application Ser. No. 63/112,486, filed Nov. 11, 2020, Provisional Application Ser. No. 63/119,878, filed Dec. 1, 2020, and Provisional Application Ser. No. 63/153,817, filed Feb. 25, 2021, the disclosures of which are incorporated herein by reference in their entirety.

### BACKGROUND

Guide RNAs that facilitate RNA editing by endogenous and exogenous RNA editing enzymes have the potential to address a wide array of diseases. However, compositions and methods that enhance RNA stability and retention are needed to quantitatively and qualitatively increase the potency of these guide RNAs.

### SUMMARY

Therapeutic opportunities are possible with the modulation of splicing activity; however, efficiency, specificity, and delivery of this approach has not performed well in a clinical setting. The disclosure provides for engineered polynucleotides that provide significant improvements in efficiency, specificity, and delivery of splicing activity modulation and RNA base editing.

Here we adapt the U7 snRNA gene to engineer a novel chassis for expressing guide RNAs for directing endogenous ADAR deaminase activity to desired gene targets. This RNA base editing can occur within coding sequences, 5′ or 3′ untranslated regions, introns; branch point adenosines, or splice acceptor sites. Such RNA base editing can result in: altering the protein coding sequence; removing a mutated premature stop codon; modulating the stability of the RNA; or altering the splicing of the pre-mRNA.

The present disclosure provides an engineered polynucleotide comprising: a targeting sequence that at least partially hybridizes to at least a portion of a target RNA and contains at least one mismatched nucleotide; an Sm or Sm-like protein binding domain or variant thereof from a spliceosomal snRNA or a non-spliceosomal small nuclear RNA (snRNA); and a hairpin from a spliceosomal snRNA or a non-spliceosomal snRNA or a variant thereof.

The present disclosure also provides an engineered polynucleotide comprising: a targeting sequence that at least partially hybridizes to at least a portion of a target RNA and contains at least one adenine-guanine (A-G) mismatch, at least one adenine-adenine (A-A) mismatch, or at least one adenine-cytosine (A-C) mismatch; an Sm or Sm-like protein binding domain, or variant thereof, from a spliceosomal snRNA or a non-spliceosomal small nuclear RNA (snRNA); a hairpin from a spliceosomal snRNA or a non-spliceosomal snRNA, or a variant thereof, wherein the engineered polynucleotide is configured to facilitate editing of a base of the target RNA by an RNA editing entity.

The mismatch can be at least one adenine-guanine (A-G) mismatch, at least one adenine-adenine (A-A) mismatch, or at least one adenine-cytosine (A-C). The targeting sequence can be at least 30-300 or 50-100 bases in length. The engineered polynucleotide can be operably linked to an RNA polymerase II-type promoter. The RNA polymerase II-type promoter is a U1 promoter. The RNA polymerase II-type promoter is a U7 promoter. The engineered polynucleotide is operably linked to a U6 promoter. The Sm or Sm-like protein binding domain, or variant thereof is a SmOPT sequence. The SmOPT sequence has at least 80% sequence identity to AAUUUUUGGAG or SEQ ID NO: 41. The SmOPT sequence is AAUUUUUGGAG or SEQ ID NO: 41. The hairpin is from a mouse U7 snRNA, a human U7 snRNA, or a human U1 snRNA. The hairpin is a chimeric hairpin of one or more of a mouse U7 snRNA, a human U7 snRNA, a human U1 snRNA. The hairpin has at least 80%, at least 85%, at least 90%, at least 92%, at least 95%, at least 97%, or at least 99% sequence identity to the hairpin sequence in any one of SEQ ID NO: 42, SEQ ID NO: 43, SEQ ID NO: 45, or SEQ ID NO: 46. The hairpin has the hairpin sequence of any one of SEQ ID NO: 42, SEQ ID NO: 43, SEQ ID NO: 45, or SEQ ID NO: 46. The hairpin has a sequence of SEQ ID NO: 43. The engineered polynucleotide can further comprise a U7 box terminator at the 3′ end of the engineered polynucleotide. The targeting sequence from 5′ to 3′ comprises the targeting sequence, the Sm or Sm-like protein binding domain or variant thereof, and the hairpin. The engineered polynucleotide is configured to facilitate editing of a base of the target RNA by an RNA editing entity. The RNA editing comprises an ADAR protein, an APOBEC protein, or both. The RNA editing entity comprises ADAR and wherein ADAR comprises ADAR1 or ADAR2. The targeting sequence at least partially binds to a target RNA that is implemented in a disease or condition. The target RNA can be selected from the group consisting of RAB7A, ABCA4, SERPINA1, SERPINA1 E342K, HEXA, LRRK2, SNCA, DMD, APP, Tau, CFTR, ALAS1, ATP7B, ATP7B G1226R, HFE C282Y, LIPA c.894 G>A, PCSK9 start site, or SCNN1A start site, a fragment any of these, and any combination thereof. The disease or condition which comprises Rett syndrome, Huntington's disease, Parkinson's Disease, Alzheimer's disease, a muscular dystrophy, or Tay-Sachs Disease. The engineered polynucleotide further comprises an additional sequence from an snRNA. An additional sequence from an snRNA can comprise at least in part a U1, U2, U4, U5, U6, or U7 snRNA sequence. A polynucleotide comprising an snRNA sequence can have at least 80% identity to any one of SEQ ID NO: 1-SEQ ID NO: 33 or a variant thereof. The targeting sequence comprises a mismatch relative to a sequence of the target RNA. The mismatch can be an A/C mismatch and wherein the targeting sequence comprises a C of the A/C mismatch and the sequence of the target RNA comprises an A of the A/C mismatch. The A/C mismatch can be configured to promote an edit in the pre-mRNA when associated with the pre-mRNA in the presence of a deaminase. The mismatch is located at least 45, 46, 47, 48, 49, 50, 51, 52, 53, 54, or 55 bases from either end of the targeting sequence. In some embodiments, the mismatch is located from 10 to 30 bases from either end of the targeting sequence. In some embodiments, the mismatch is located about 20 bases from either end of the targeting sequence. In some embodiments, the mismatch is located exactly 20 bases from either end of the targeting sequence. In some embodiments, the mismatch is located about 70 to 90 bases from either end of the targeting sequence. In some embodiments, the mismatch is located about 80 bases from either end of the targeting sequence. In some embodiments, the mismatch is located exactly 80 bases from either end of the targeting sequence. The snRNA promoter comprises at least in part a U1, U2, U4, U5, U6, or U7 snRNA promoter.

The targeting sequence can be at least partially complementary to a splice signal proximal to an exon within the target RNA. The targeting sequence is: (a) at least partially complementary to a branch point upstream of an exon within the target RNA; or (b) the targeting sequence is at least partially complementary to a donor splice site downstream of an exon within the target RNA.

The engineered polynucleotide can have improved efficiency of exon skipping as compared to a comparable exon skipping construct without the Sm or Sm-like binding domain or variant thereof, without the hairpin, or without the Sm or Sm-like binding domain or variant thereof and the hairpin when measured in vitro. The efficiency can be determined by performing a droplet digital PCR assay to detect a percent skipping of the exon skipped by the exon skipping guide RNA construct in a cell transfected by the editing construct relative to a cell comprising the editing construct without the mismatched nucleotide. The engineered polynucleotide can be configured to facilitate an edit of a base within the splice signal. The edit can be configured to promote skipping of an exon. The engineered polynucleotide has increased efficiency of exon skipping of at least 1%, at least 5%, at least 10%, at least 20%, at least 30%, at least 40%, or at least 50% as measured by an in vitro assay. The nucleotide that is not complementary to the pre-mRNA within the region targeting the pre-mRNA configures the engineered polynucleotide to recruit a deaminase when associated with the target mRNA. The targeting sequence when bound to the pre-mRNA and in association with a deaminase facilitates a chemical modification of a base of a polynucleotide of the pre-mRNA by the deaminase. The engineered polynucleotide further comprises a deaminase recruiting domain.

The deaminase recruiting domain can be selected from the group consisting of at least a portion of: GluR2, Alu, a variant of either of these, and any combination thereof. The deaminase recruiting domain comprises a stem loop. The stem loop is a left-handed stem loop or a right-handed stem loop. The stem loop comprises at least about 80% sequence identity to a GluR2 domain. The engineered polynucleotide comprises at least one chemically modified nucleotide. The chemical modification comprises at least one modification of one or both of non-linking phosphate oxygen atoms in a phosphodiester backbone linkage of the engineered polynucleotide as provided in Table 1.

The engineered polynucleotide, when present in an aqueous solution and not bound to the target RNA, lacks at least one of a hairpin, a bulge, a polynucleotide loop, a structured domain, or any combination thereof. For example, one of the aforementioned structural features, a hairpin, a bulge, a polynucleotide loop, a structured domain, or any combination thereof, may form in a duplex RNA formed by binding of an engineered polynucleotide of the present disclosure to a target RNA. The structural feature comprises a bulge, an internal loop, a hairpin, or any combination thereof. The structural feature is a bulge. The bulge is an asymmetric bulge. The bulge is a symmetric bulge. The structural feature is an internal loop. The internal loop is an asymmetric loop. The internal loop is a symmetric loop. The engineered polynucleotide comprises a structural loop stabilized scaffold. The structural loop stabilized scaffold further comprises a targeting sequence that, in association with the target RNA, recruits an RNA editing enzyme, wherein the RNA editing enzyme chemically modifies the base of the nucleotide of the target RNA; and wherein the targeting sequence comprises more than or equal to about 4 contiguous nucleotides. The engineered polynucleotide further comprises a first spacer domain and a second spacer domain flanking the targeting sequence, wherein the engineered polynucleotide is configured to self-circularize after transcription in a mammalian cell. The engineered polynucleotide comprises a ribozyme domain 5′ to the first spacer domain or 3′ to the second spacer domain. The engineered polynucleotide comprises a ligation domain between the ribozyme domain and the first spacer domain or between the ribozyme domain and the second spacer domain. The engineered polynucleotide is linear, when the polynucleotide sequence is represented 2-dimensionally. The engineered polynucleotide is circular, when the polynucleotide sequence is represented 2-dimensionally. The engineered polynucleotide is configured to be circularized in situ in a mammalian cell. The targeting sequence does not comprise an aptamer. The engineered polynucleotide does not comprise or encode a sequence encoding an sequence configured for RNA interference (RNAi). The targeting sequence is configured to at least partially associate with at least a portion of a 3′ or 5′ untranslated region (UTR) of the target RNA. The targeting sequence is configured to at least partially associate with at least a portion of an intronic region of the target RNA. The targeting sequence is configured to at least partially associate with at least a portion of an exonic region of the target RNA. The engineered polynucleotide is about 80 nucleotides to about 600 nucleotides. The engineered polynucleotide comprises a first spacer domain 5′ to the targeting sequence. The engineered polynucleotide comprises a second spacer domain distinct from or identical to the first spacer domain. The first spacer domain, the second spacer domain or both comprises a polynucleotide sequence of: AUAUA (SEQ ID NO: 53). The first spacer domain, the second spacer domain or both comprises a polynucleotide sequence of: AUAAU (SEQ ID NO: 52). The engineered polynucleotide comprises a first ribozyme domain on a 5′ end and a second ribozyme domain on a 3′ end. The first or second ribozyme is independently selected from the group consisting of a Hammerhead ribozyme, a glmS ribozyme, an HDV-like ribozyme, an R2 element, a peptidyl transferase 23S rRNA, a GIR1 branching ribozyme, a leadzyme, a group II intron, a hairpin ribozyme, a VS ribozyme, a CPEB3 ribozyme, a CoTC ribozyme, and a group I intron

Provided herein, is a vector comprising or encoding the engineered polynucleotide of the present disclosure. The vector comprises a liposome, a nanoparticle, or a dendrimer. The vector is a viral vector. The viral vector is an adeno-associated viral (AAV) vector. The AAV vector is an AAV2 vector, AAV5 vector, AAV8 vector, AAV9 vector, or a hybrid of any of these. The viral vector is a self-complementary adeno-associated viral (scAAV) vector. The viral vector is a single-stranded AAV vector.

Provided herein is an isolated cell comprising the engineered polynucleotide of the present disclosure, the polynucleotide encoding the engineered polynucleotide of the present disclosure, or the vector of the present disclosure. The isolated cell can be a T cell, a neuron, a myocyte, a hepatocyte, and the like.

Provided herein is a pharmaceutical composition in unit dose form comprising the engineered polynucleotide of the present disclosure, a polynucleotide encoding engineered polynucleotide of the present disclosure, or the vector of the present disclosure; and a pharmaceutically acceptable: excipient, diluent, or carrier.

Provided herein is a method of treating or preventing a condition in a subject in need thereof, comprising administering to the subject the engineered polynucleotide of the present disclosure, a polynucleotide encoding engineered polynucleotide of the present disclosure, the vector of the present disclosure, or the pharmaceutical composition of the present disclosure. The condition can be Duchenne's Muscular Dystrophy (DMD), Rett's syndrome, Charcot-Marie-Tooth disease, Alzheimer's disease, Parkinson's disease, alpha-1 anti trypsin deficiency, or Stargardt's disease. The condition is associated with a mutation in a protein selected from the group consisting of RAB7A, ABCA4, SERPINA1, SERPINA1 E342K, HEXA, LRRK2, SNCA, DMD, APP, Tau, CFTR, ALAS1, ATP7B, ATP7B G1226R, HFE C282Y, LIPA c.894 G>A, PCSK9 start site, or SCNN1A start site, a fragment any of these, and any combination thereof. The administering can be parental. The administering can be a parenchymal injection, an intra-thecal injection, an intra-ventricular injection, an intra-cisternal injection, an intravenous injection, an intramuscular injection, intracerbroventricular (ICV) administration, subcutaneous injection, oral administration, mucosal administration, sublingual administration, buccal administration, rectal administration, ocular administration, otic administration, nasal administration, topical administration, cutaneous administration, transdermal administration, or any combination thereof.

The method of the present disclosure can further comprise administering an additional treatment. The additional treatment can be administered concurrently or consecutively. The administering can be performed at least once a week. The subject has been diagnosed with the disease or condition by an in vitro diagnostic prior to the administering. The subject can be human, and optionally the subject is male or female, and the subject is optionally an adult, or the subject is optionally less than 18 years of age.

Provided herein is a kit comprising the engineered polynucleotide of the present disclosure in a container, a polynucleotide encoding engineered polynucleotide of the present disclosure in a container, the vector of the present disclosure in a container, or the pharmaceutical composition of the present disclosure in a container.

Provided herein is a method of making a pharmaceutical composition, comprising contacting a pharmaceutically acceptable: excipient, carrier, or diluent with at least one of the engineered polynucleotide of the present disclosure, the polynucleotide encoding engineered polynucleotide of the present disclosure, or the vector of the present disclosure.

Provided herein is a method of making a kit, comprising placing at least in part, into a container: 1) the engineered polynucleotide of the present disclosure; 2) a polynucleotide encoding engineered polynucleotide of the present disclosure; 3) the vector of the present disclosure; or 4) the pharmaceutical composition of the present disclosure.

### DESCRIPTION OF DRAWINGS

FIG. 1 shows an example assay for assessment of exon skipping of DMD Exon 71 and exon 74. The arrows denote primers and the solid lines denote the probe, for use in either Quantitative PCR or droplet digital PCR. The below panels show representative data of a droplet digital PCR exon skipping assay performed on extracted cDNA derived from human skeletal muscle.

FIGS. 2A-2B show U7/U1 promoters enhance specific guide RNA editing in conjunction with a 3′ hairpin. 100 nt guide RNAs targeting the human RAB7A 3′UTR, human DMD exon 71 Splice Acceptor, or human DMD exon 74 Splice Acceptor were expressed using the hU6, mU7, hU7, or hU1 promoters with or without a corresponding 3′ hairpin. RNA was measured 38 hours later. FIG. 2A shows engineered polynucleotides with a promoter sequence (U7 or U1), combined with a 3′ hairpin (SmOPT U7 or hU1), effect RAB7A ADAR editing and DMD exon 71 or 74 skipping (measured by ddPCR). The white bars indicate experiments where cells were transfected with a GFP expressing vector, whereas the solid bar indicates transfection with an ADAR2 overexpression vector. Dots represent two independent experiments with the bar graph representing the average of the two experiments. FIG. 2B shows Sanger sequencing chromatograms of specific editing at the target adenosine in the RAB7A 3′ UTR (box). Since a reverse primer was used for sequencing, an A>G edit appears as T>C.

FIGS. 3A-3D show U7 promoter-driven expression of engineered polynucleotides with a 3′ SmOPT and U7 hairpin enhances specific guide RNA editing at additional gene targets with minimal unintended exon skipping. FIG. 3A shows the exon structure of human RAB7A and SNCA. Exons are shown as gray segments; the coding region is denoted as a black line above. Locations of the guide RNA targeting sites are shown; PCR primers are also shown. FIG. 3B shows ADAR editing at each target site (measured by Sanger sequencing). FIG. 3C shows cDNA from edited transcripts that was PCR amplified using the above primers and analyzed on an agarose gel. PCR amplicons showed the predicted size for correctly spliced exons. FIG. 3D shows Sanger sequencing chromatograms revealing specific editing at the target adenosine of the indicated transcripts (red box).

FIG. 4 shows editing of the SNCA start codon (Translation Initiation Site) and SNCA 3′UTR in additional cell types at multiple time points. Engineered polynucleotides targeting the human SNCA start codon or human SNCA 3′UTR were expressed using a hU6 promoter with no hairpin, a mU7 promoter with a 3′ SmOPT mU7 hairpin, a hU7 promoter with a 3′ SmOPT hU7 hairpin, or a hU1 promoter with a 3′ SmOPT mU7 hairpin. FIG. 4A shows Sanger sequencing chromatograms revealing base editing in HEK 293 cells, with or without ADAR2 overexpression, at the on-target site (red box) along with additional off-target editing sites. The percent of editing is noted below each peak. FIG. 4B shows the percent of editing of the SNCA 3′ UTR in K562 cells which overexpress SNCA, under different transfection conditions (nucleofection, Lonza) at the indicated time points. Cells were transfected with a GFP expressing vector (endogenous ADAR, open symbols) or an ADAR2 overexpression vector (solid symbols).

FIG. 5 shows that a human U1 promoter can be paired with a 3′ SmOPT sequence and U7 hairpin for guide RNA editing with minimal knockdown of transcript levels. Guide RNAs targeting the human RAB7A 3′UTR, human DMD exon 71 Splice Acceptor, or human DMD exon 74 Splice Acceptor were expressed using the hU6, mU7, hU7, or hU1 promoters with a 3′ SmOPT U7 hairpin and U7 termination sequence. Alternatively, these 100 nt guide RNAs were expressed using the hU6, mU7, hU7, or hU1 promoters with circularizing RtcB ribozyme sites from Litke and Jaffrey 2019 (no SmOPT or U7 hairpin). RNA was measured at the indicated time point post transfection by ddPCR.

FIG. 6 shows that adding hnRNP A1 binding domains [UAGGGW] at the 5′ end of the guide RNA opposite a 3′ SmOPT U7 hairpin affects RAB7A editing and DMD exon 74 skipping. Guide RNAs targeting the human RAB7A 3′UTR, human DMD exon 71 Splice Acceptor, or human DMD exon 74 Splice Acceptor were expressed using the mU7 or hU1 promoters with a 3′ SmOPT mU7 hairpin and mU7 termination sequence. Alternatively, these 100 nt guide RNAs were expressed using the hU6 promoter with circularizing RtcB ribozyme sites from Litke and Jaffrey 2019 (no SmOPT or U7 hairpin). Added at the 5′ end of the guide RNA was either: no tag; a triple hnRNP A1 binding domain (Dickson 2008); a double hnRNP A1 binding domain; or a mutated domain that does not bind hnRNP A1 as a negative control. RNA was measured 42 hr later. FIG. 6A shows the hnRNP binding domains, when combined with the U7/U1 promoters and a 3′ SmOPT U7 hairpin, increased RAB7A ADAR editing and DMD exon 74 skipping (measured by ddPCR). FIG. 6B shows Sanger sequencing chromatograms showing specific editing at the target adenosine in the RAB7A 3′UTR (box). Since a reverse primer was used for sequencing, an A>G edit appears as T>C.

FIG. 7 shows that the SmOPT sequence is required for full editing, but that a U7 hairpin from mouse, human, or a hybrid mouse/human combination can suffice for editing. Guide RNAs targeting 100 nt of the human RAB7A exon 4, RAB7A 3′UTR, SNCA exon 4, SNCA 3′UTR, DMD exon 71 Splice Acceptor, or DMD exon 74 Splice Acceptor were expressed using the mU7 promoter with a variation of the SmOPT domain or a variation of the U7 hairpin along with a mU7 termination sequence. FIG. 7A lists the sequence variations of the Sm domain and U7 hairpin. The SmOPT sequence was replaced by a natural Sm binding domain from the U1 or U7 snRNA or a mutated version which does not bind Sm. Alternatively, the mU7 hairpin was replaced by the hU7 hairpin or a hybrid mouse/human combination. FIG. 7B shows that guide RNA's containing a SmOPT sequence, but not a variation, can produce robust editing. FIG. 7C shows Sanger sequencing chromatograms showing specific editing at the target adenosines of the indicated transcripts (red box).

FIG. 8 shows constructs of piggyBac vectors carrying a LRRK2 minigene having a G2019S mutation and mCherry (at top) or a carrying a LRRK2 minigene having a G2019S mutation, mCherry, CMV, and ADAR2 (at bottom).

FIG. 9A shows in vitro on and off-target editing of the LRRK2 G2019S mutation by ADAR1 after administration of two guide RNAs and a control (GFP plasmid). FIG. 9B shows in vitro on and off-target editing of the LRRK2 G2019S mutation by ADAR1 and ADAR2 after administration of two guide RNAs and a control (GFP plasmid).

FIG. 10 shows that a guide RNA containing a 3′ SmOPT sequence and U7 hairpin can be circularized and expressed by U7 or U6 promoters to produce ADAR editing. FIG. 10A illustrates a 100 nt guide RNA with or without a 3′ SmOPT U7 hairpin flanked by RtcB circular ribozyme sites. Sanger sequencing with a guide-specific primer (black) shows that the ribozyme sites have been successfully joined together, with the guide RNA and 3′ SmOPT U7 hairpin present inside the circular RNA. FIG. 10B compares different variations of the SmOPT U7 circular guide RNA using either the mU7 or hU6 promoter, different Sm binding domains and U7 hairpins, and various length linkers between the U7 hairpin and P1 circular ribozyme (upper panel). As above, a linear 100 nt guide RNA with a 3′ SmOPT sequence and U7 hairpin could cause ADAR RNA editing all six gene targets: human RAB7A exon 4, RAB7A 3′UTR, SNCA exon 4, SNCA 3′UTR, DMD exon 71 Splice Acceptor, or DMD exon 74 Splice Acceptor (middle panel). Circular variations of a 100 nt guide RNA with a 3′ SmOPT sequence and U7 hairpin could also generate substantial editing, whether expressed by the mU7 or hU6 promoters. Side effects of target transcript knockdown or inadvertent exon skipping were minimal (bottom panel).

FIG. 11A shows percent RNA editing for constructs encoding for a guide RNA targeting a mutation in ABCA4, an SmOPT sequence, and a U7 hairpin, where expression is driven by a U1 promoter. FIG. 11B shows Sanger sequencing traces for the various constructs shown in FIG. 11A.

FIG. 12A shows percent RNA editing in cells by ADAR1 and ADAR2 for multiple doses of constructs encoding for a guide RNA targeting a mutation in ABCA4, an SmOPT sequence, and a U7 hairpin, where expression is driven by a U1 promoter. FIG. 12B shows percent RNA editing in cells by ADAR1 for multiple doses of constructs encoding for a guide RNA targeting a mutation in ABCA4, an SmOPT sequence, and a U7 hairpin, where expression is driven by a U1 promoter.

FIG. 13 show a plot of RNA editing of the guide RNA listed as SEQ ID NO: 31 at the target A to be edited (“0” on the x-axis) and at RNA editing at off-target positions (represented as black bars at positions that are not “0”).

FIG. 14 show a plot of RNA editing of the guide RNA listed as SEQ ID NO: 32 at the target A to be edited (“0” on the x-axis) and at RNA editing at off-target positions (represented as black bars at positions that are not “0”).

FIG. 15 show a plot of RNA editing of the guide RNA listed as SEQ ID NO: 33 at the target A to be edited (“0” on the x-axis) and at RNA editing at off-target positions (represented as black bars at positions that are not “0”).

FIG. 16 shows editing of the human SOD1 start codon where the engineered polynucleotide expression construct was delivered to HEK 293T cells either by plasmid transient transfection, or integrated into the genome as a single copy. Engineered polynucleotides targeting the human SOD1 start codon were expressed using a hU6 promoter with circularizing RtcB ribozyme sites (no SmOPT or U7 hairpin); a mU7 promoter with a 3′ SmOPT mU7 hairpin; or a mU7 promoter with a 5′ double hnRNP A1 binding domain and 3′ SmOPT hU7 hairpin.

FIG. 17 shows editing of RAB7A in muscle cells using guide RNAs expressed using a hU1 promoter with a 3′ SmOPT hU7 hairpin.

FIG. 18 shows editing of SERPINA1 minigenes 1 and 2 using guide RNAs expressed using a U6 or U7 promoter with a 3′ SmOPT hU7 hairpin.

FIG. 19 shows a plot of RNA editing of SERPINA1 for the guide RNAs listed as SEQ ID NO: 26 and SEQ ID NO: 27 at the target A to be edited (“0” on the x-axis) and at RNA editing at off-target positions (represented as black bars at positions that are not “0”)

FIG. 20 shows editing of RAB7A in LHCN muscle cells using guide RNAs with a 3′ SmOPT hU7 hairpin.

FIG. 21A and FIG. 21B shows editing of Rab7a using guide RNA's expressed using an AAV vector as a function of dose, as determined by ddPCR (FIG. 21A) and Sanger sequencing (FIG. 21B) after 9 days of differentiation. FIG. 21C shows editing of Rab7a using guide RNA's expressed using an AAV vector as a function of dose, as determined by ddPCR after 17 or more days of differentiation

FIG. 22A depicts the % transduction plotted against the Rab7 editing efficiency after 9 days of differentiation. FIG. 22B depicts the % transduction plotted against the Rab7 editing efficiency in cells harvested after varying days of differentiation.

FIG. 23 shows off-target editing profiles for the U7 smOPT linear guide relative to control.

### DETAILED DESCRIPTION OF THE DISCLOSURE

The term “a” and “an” refers to one or to more than one (e.g., to at least one) of the grammatical object of the article. By way of example, “an element” means one element or more than one element.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” meaning up to an acceptable error range for the particular value should be assumed. Also, where ranges, subranges, or both, of values can be provided, the ranges or subranges can include the endpoints of the ranges or subranges. The terms “substantially”, “substantially no”, “substantially free”, and “approximately” can be used when describing a magnitude, a position or both to indicate that the value described can be up to a reasonable expected range of values. For example, a numeric value can have a value that can be +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein can be intended to include all sub-ranges subsumed therein.

The term “partially”, “at least partially”, or as used herein can refer to a value approaching 100% of a given value. In some cases, the term can refer to an amount that can be at least about 60%, 65%, 70%, 75%, 80%, 85%, 90%, 91%, 92%, 93%, 94%, 95%, 96%, 97%, 98%, 99%, 99.9%, or 99.99% of a total amount. In some cases, the term can refer to an amount that can be about 100% of a total amount.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this disclosure. Embodiments defined by each of these transition terms are within the scope of this disclosure.

The term “subject,” “host,” “individual,” and “patient” are as used interchangeably herein to refer to animals, typically mammalian animals. Any suitable mammal can be treated by a method, cell or composition described herein. A mammal can be administered a vector, an engineered polynucleotide, a precursor guide RNA, a nucleic acid, or a pharmaceutical composition, as described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In some embodiments a subject is a human. In some embodiments, a subject has or is suspected of having a disease such as a neurodegenerative disease. In some embodiments, a subject has or can be suspected of having a cancer or neoplastic disorder. In other embodiments, a subject has or can be suspected of having a disease or disorder associated with aberrant protein expression. In some cases, a human can be more than about: 1 day to about 10 months old, from about 9 months to about 24 months old, from about 1 year to about 8 years old, from about 5 years to about 25 years old, from about 20 years to about 50 years old, from about 1 year old to about 130 years old or from about 30 years to about 100 years old. Humans can be more than about: 1, 2, 5, 10, 20, 30, 40, 50, 60, 70, 80, 90, 100, 110, or 120 years of age. Humans can be less than about: 1, 2, 5, 10, 20, 30, 40, 50, 60, 70, 80, 90, 100, 110, 120 or 130 years of age.

The term “subject,” “host,” “individual,” and “patient” are as used interchangeably herein to refer to animals, typically mammalian animals. Any suitable mammal can be administered a composition as described herein (such as an engineered polynucleotides) or treated by a method as described herein. A subject can be a vertebrate or an invertebrate. A subject can be a laboratory animal. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. In some embodiments a subject is a human. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

“Homology” or “identity” or “similarity” can refer to sequence similarity between two peptides or between two nucleic acid molecules. Homology can be determined by comparing a position in each sequence which can be aligned for purposes of comparison. When a position in the compared sequence can be occupied by the same base or amino acid, then the molecules can be homologous at that position. A degree of homology between sequences can be a function of the number of matching or homologous positions shared by the sequences. An “unrelated” or “non-homologous” sequence shares less than 40% identity, or alternatively less than 25% identity, with one of the sequences of the disclosure. Sequence homology can refer to a % identity of a sequence to a reference sequence. As a practical matter, whether any particular sequence can be at least 50%, 60%, 70%, 80%, 85%, 90%, 92%, 95%, 96%, 97%, 98% or 99% identical to any sequence described herein (which can correspond with a particular nucleic acid sequence described herein), such particular polypeptide sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence, the parameters can be set such that the percentage of identity can be calculated over the full length of the reference sequence and that gaps in sequence homology of up to 5% of the total reference sequence can be allowed.

In some cases, the identity between a reference sequence (query sequence, e.g., a sequence of the disclosure) and a subject sequence, also referred to as a global sequence alignment, can be determined using the FASTDB computer program. In some embodiments, parameters for a particular embodiment in which identity can be narrowly construed, used in a FASTDB amino acid alignment, can include: Scoring Scheme=PAM (Percent Accepted Mutations) 0, k-tuple=2, Mismatch Penalty=1, Joining Penalty=20, Randomization Group Length=0, Cutoff Score=1, Window Size=sequence length, Gap Penalty=5, Gap Size Penalty=0.05, Window Size=500 or the length of the subject sequence, whichever can be shorter. According to this embodiment, if the subject sequence can be shorter than the query sequence due to N- or C-terminal deletions, not because of internal deletions, a manual correction can be made to the results to take into consideration the fact that the FASTDB program does not account for N- and C-terminal truncations of the subject sequence when calculating global percent identity. For subject sequences truncated at the N- and C-termini, relative to the query sequence, the percent identity can be corrected by calculating the number of residues of the query sequence that can be lateral to the N- and C-terminal of the subject sequence, which can be not matched/aligned with a corresponding subject residue, as a percent of the total bases of the query sequence. A determination of whether a residue can be matched/aligned can be determined by results of the FASTDB sequence alignment. This percentage can be then subtracted from the percent identity, calculated by the FASTDB program using the specified parameters, to arrive at a final percent identity score. This final percent identity score can be used for the purposes of this embodiment. In some cases, only residues to the N- and C-termini of the subject sequence, which can be not matched/aligned with the query sequence, can be considered for the purposes of manually adjusting the percent identity score. That is, only query residue positions outside the farthest N- and C-terminal residues of the subject sequence can be considered for this manual correction. For example, a 90-residue subject sequence can be aligned with a 100-residue query sequence to determine percent identity. The deletion occurs at the N-terminus of the subject sequence, and therefore, the FASTDB alignment does not show a matching/alignment of the first 10 residues at the N-terminus. The 10 unpaired residues represent 10% of the sequence (number of residues at the N- and C-termini not matched/total number of residues in the query sequence) so 10% can be subtracted from the percent identity score calculated by the FASTDB program. If the remaining 90 residues were perfectly matched, the final percent identity can be 90%. In another example, a 90-residue subject sequence can be compared with a 100-residue query sequence. This time the deletions can be internal deletions, so there can be no residues at the N- or C-termini of the subject sequence which can be not matched/aligned with the query. In this case, the percent identity calculated by FASTDB can be not manually corrected. Once again, only residue positions outside the N- and C-terminal ends of the subject sequence, as displayed in the FASTDB alignment, which can be not matched/aligned with the query sequence can be manually corrected for.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA of a sequence, isolated RNA of a sequence, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA). A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double and single stranded molecules. Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction. Unless otherwise specified or required, any embodiment of this disclosure that is a polynucleotide encompasses both the double stranded form and each of two complementary single stranded forms known or predicted to make up the double stranded form.

The term “guide RNA” can be used interchangeably with the terms “engineered polynucleotide,” “polynucleotide,” “oligonucleotide,” etc. to refer to the polynucleotides of the present disclosure.

Polynucleotides useful in the methods of the disclosure can comprise natural nucleic acid sequences and variants thereof, artificial nucleic acid sequences, or a combination of such sequences.

A polynucleotide is composed of a specific sequence of four nucleotide bases: adenine (A); cytosine (C); guanine (G); thymine (T); and uracil (U) for thymine when the polynucleotide is RNA. Any sequence described herein can be DNA; where the sequence is transcribed into RNA, the thymine (T) can be replaced with a uracil (U). In some instances, an RNA sequence described herein can be represented as a DNA sequence with a T in place of a U. In some embodiments, the polynucleotide may comprise one or more other nucleotide bases, such as inosine (I), a nucleoside formed when hypoxanthine is attached to ribofuranose via a β-N9-glycosidic bond, resulting in the chemical structure:

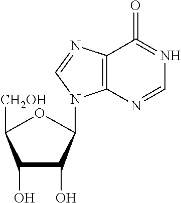

Inosine is read by the translation machinery as guanine (G).

The term “polynucleotide sequence” is the alphabetical representation of a polynucleotide molecule. This alphabetical representation can be input into databases in a computer having a central processing unit and used for bioinformatics applications such as functional genomics and homology searching.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The terms “equivalent” or “biological equivalent” are used interchangeably when referring to a particular molecule, biological, or cellular material and intend those having minimal homology while still maintaining desired structure or functionality.

“Droplet digital PCR” (ddPCR) refers to a digital PCR assay that measures absolute quantities by counting nucleic acid molecules encapsulated in discrete, volumetrically defined, water-in-oil droplet partitions that support PCR amplification. A single ddPCR reaction may contain at least 20,000 partitioned droplets per well.

A “droplet” or “water-in-oil droplet” refers to an individual partition of the droplet digital PCR assay. A droplet supports PCR amplification of template molecule(s) using homogenous assay chemistries and workflows similar to those widely used for real-time PCR applications.

Droplet digital PCR may be performed using any platform that performs a digital PCR assay that measures absolute quantities by counting nucleic acid molecules encapsulated in discrete, volumetrically defined, water-in-oil droplet partitions that support PCR amplification. The strategy for droplet digital PCR may be summarized as follows: a sample is diluted and partitioned into thousands to millions of separate reaction chambers (water-in-oil droplets) so that each contains one or no copies of the nucleic acid molecule of interest. The number of “positive” droplets detected, which contain the target amplicon (e.g., nucleic acid molecule of interest), versus the number of “negative” droplets, which do not contain the target amplicon (e.g., nucleic acid molecule of interest), may be used to determine the number of copies of the nucleic acid molecule of interest that were in the original sample. Examples of droplet digital PCR systems include the QX100™ Droplet Digital PCR System by Bio-Rad, which partitions samples containing nucleic acid template into 20,000 nanoliter-sized droplets; and the RainDrop™ digital PCR system by RainDance, which partitions samples containing nucleic acid template into 1,000,000 to 10,000,000 picoliter-sized droplets.

The term “mutation” as used herein, refers to an alteration to a nucleic acid sequence encoding a protein relative to the consensus sequence of said protein. “Missense” mutations result in the substitution of one codon for another; “nonsense” mutations change a codon from one encoding a particular amino acid to a stop codon. Nonsense mutations often result in truncated translation of proteins. “Silent” mutations are those which have no effect on the resulting protein. As used herein the term “point mutation” refers to a mutation affecting only one nucleotide in a gene sequence. “Splice site mutations” are those mutations present pre-mRNA (prior to processing to remove introns) resulting in mistranslation and often truncation of proteins from incorrect delineation of the splice site. A mutation can comprise a single nucleotide variation (SNV). A mutation can comprise a sequence variant, a sequence variation, a sequence alteration, or an allelic variant. The reference DNA sequence can be obtained from a reference database. A mutation can affect function. A mutation may not affect function. A mutation can occur at the DNA level in one or more nucleotides, at the ribonucleic acid (RNA) level in one or more nucleotides, at the protein level in one or more amino acids, or any combination thereof. The reference sequence can be obtained from a database such as the NCBI Reference Sequence Database (RefSeq) database. Specific changes that can constitute a mutation can include a substitution, a deletion, an insertion, an inversion, or a conversion in one or more nucleotides or one or more amino acids. A mutation can be a point mutation. A mutation can be a fusion gene. A fusion pair or a fusion gene can result from a mutation, such as a translocation, an interstitial deletion, a chromosomal inversion, or any combination thereof. A mutation can constitute variability in the number of repeated sequences, such as triplications, quadruplications, or others. For example, a mutation can be an increase or a decrease in a copy number associated with a given sequence (i.e., copy number variation, or CNV). A mutation can include two or more sequence changes in different alleles or two or more sequence changes in one allele. A mutation can include two different nucleotides at one position in one allele, such as a mosaic. A mutation can include two different nucleotides at one position in one allele, such as a chimeric. A mutation can be present in a malignant tissue. A presence or an absence of a mutation can indicate an increased risk to develop a disease or condition. A presence or an absence of a mutation can indicate a presence of a disease or condition. A mutation can be present in a benign tissue. Absence of a mutation may indicate that a tissue or sample is benign. As an alternative, absence of a mutation may not indicate that a tissue or sample is benign. Methods as described herein can comprise identifying a presence of a mutation in a sample.

DNA is transcribed by RNA polymerases to synthesize a pre-mRNA transcript containing sequences that are important for RNA stability and regulatory function. In the case of coding RNAs (mRNAs), pre-mRNAs are subjected to RNA processing in order to convert a pre-mRNA into mature mRNA through alternative splicing of exons. Mature mRNA is subsequently translated into a protein by the ribosome. Mutations in genomic DNA can be present, arising mutations in the pre-mRNA transcript, affecting protein function or expression of said protein. These mutations, if present at splicing sites, can prevent proper alternative splicing by affecting selection of affected exons. Specifically, the pre-mRNA splicing sites contain conserved splice acceptor and splice donor sites. A splice donor site is characterized by a nucleotide motif, N/GT, wherein N is any nucleotide, and “/” represents the exon-intron junction. A splice acceptor site is characterized by the motif, NAG/NN, wherein N represents any nucleotide, and the “I” denotes the exon-intron junction. Mutations that remove or introduce this motif into intron and exon junctions can cause aberrant mRNA splicing leading to improper protein production.

Disclosed herein are compositions and methods for the editing of mRNA using a polynucleotide exon skipping construct capable of facilitating editing of a target RNA via a deaminase.

RNA splicing is a highly regulated process requiring many proteins to participate in RNA processing in mammals. RNA splicing is a form of RNA processing in which a pre-mRNA transcript is spliced into a mature mRNA via the joining of exon and removal of intervening exons. The mature mRNA is then destined for translation by the ribosomal machinery. Distinct base sequences in the pre-mRNA transcript define the exonic and intronic boundaries to facilitate proper splicing and subsequent expression of proteins and their isoforms. However, mutations present in genomic DNA can be present in a pre-mRNA transcript, preventing proper splicing of the pre-mRNA into a functional protein product. These mutations cause incorrect joining of exons, leading to non-functional proteins, potentially leading to disease. Alternatively, a mutation present in genomic DNA may retain proper splicing of the pre-mRNA, but instead be rescued by an intervention leading to incorrect joining of the exons. Examples of such diseases include Duchenne muscular dystrophy, Spinal Muscular Atrophy, cystic fibrosis, Beta thalassaemia (β-globin), Hurler syndrome, and Dravet Syndrome

Ribonucleic acids (RNA) are transcribed from genomic DNA in the cell nucleus. After transporting out of the nucleus, RNA is translated by ribosomes to produce functional proteins to carry out biological processes. In eukaryotic systems, RNA is synthesized initially as pre-mRNA, wherein the pre-mRNA is subjected to RNA processing, such as alternative splicing, in order to produce mature RNA that is ready for translation by the ribosome.

Alternative splicing is the process in which pre-mRNA is processed in order to generate mature RNA. Pre-mRNA is comprised of exons and introns, as well as other untranslated regions (3′ and 5′ UTR). During alternative splicing, splicing signals demarcating the exons and introns of the pre-mRNA enable the spliceosome to join multiple exons together to form a functional protein, and thereby removing the interceding introns.

The presence of “splicing signals” on an expression vector often results in higher levels of expression of the recombinant transcript. Splicing signals mediate the removal of introns from the primary RNA transcript and consist of a splice donor and acceptor site.

Recent work has highlighted the possibility of utilizing methods to induce exon skipping of a protein coding transcript. In many cases, a number of proteins such as alpha-synuclein and DMD can be expressed as different splice variants, some of which can be implicated in disease. It is thought that by promoting exon skipping events, exons containing a mutation implicated in a disease can be bypassed, or a codon reading frame can be restored, thereby facilitating the translation of variants that are sufficient to correct a disease or disorder, or alleviate symptoms of a disease or disorder

Guide RNAs can be expressed using the human or mouse U6 snRNA promoter. RNA polymerase III-type promoters, like U6, typically transcribe short “housekeeping” RNAs. U6 promoters can also be used to express single guide RNAs for Crispr/Cas9 nucleases and short hairpin RNAs for RNA interference.

SnRNA promoters (besides U6) are RNA polymerase II-type promoters. Like those used for mRNA expression, snRNA transcripts can be processed differently from mRNA (no polyadenylation, different 5′ capping). The polymerase can associate with an integrator complex. In some cases, sequence elements in both the promoter and 3′ terminator may need to be recognized.

An SmOPT sequence can be represented as AAUUUUUGGAG or AATTTTTGGAG (SEQ ID NO: 41) if represented with thymine in place of uracil. Certain nucleotides of the U7 snRNA (which naturally hybridizes with the spacer element of histone pre-mRNA) have been replaced with antisense sequences (e.g. against Beta-globin pre-mRNA).

Endogenous U7 snRNA can be expressed at a low level, approximately 2 to 15×10³ molecules per cell. However, the expression level and the nuclear concentration of U7 snRNA can be increased significantly by converting the wild-type U7 Sm-binding site (AAUUUGUCUAG) to the consensus Sm-binding sequence derived from the major spliceosomal snRNPs (SmOPT, AAUUUUUGGAG) as described herein. Moreover, this SmOPT modification of U7 snRNA as described herein can reduce histone pre-mRNA processing. This potentially has two beneficial effects: (i) the target RNA may not be cleaved by the histone 3′ end processing machinery, and (ii) the RNA may not compete with endogenous U7 snRNP for potentially limiting U7-specific proteins. Finally, a guide RNA with an SmOPT modification as described herein may be redirected to the sites of pre-mRNA splicing.

In some instances, a modified U7 snRNA construct under a U1 promoter can be used (i.e. a hybrid with a modified U7 snRNA gene under the control of the U1 promoter and terminator sequences).

A modified SmOPT U7 snRNA chassis may be used with antisense sequences of other genes to modify RNA splicing for other diseases as described herein. For example, U7 antisense oligo constructs for exon skipping can include binding sites for hnRNP A1. When hnRNP A1 is recruited to splice sites, it can block splicing machinery and promote exon skipping.

In some diseases, such as Duchenne muscular dystrophy (DMD), an exon of a protein may possess a mutation which causes disease when expressed. Inclusion of exons with mutations that cause disease have been explored for therapeutic intervention. Exon skipping has been explored as a method in which to prevent the mutated exon from being selected for during alternative splicing events. The in-frame exons 71 and 74 can contain Becker's and Duchenne's mutations, respectively.

The ability to skip an exon has proven to be an attractive solution to prevent the use of an exon that would cause disease. Previous attempts on using exon skipping have utilized reagents that mask the splicing signals in order to facilitate exon exclusion or exon inclusion for therapeutic purposes.

### Engineered Polynucleotide Constructs for RNA Editing

Disclosed herein are compositions and methods for the editing of mRNA using an engineered polynucleotide having snRNA sequences and snRNA hairpins (e.g., SmOPT sequences and U7 hairpins or variants thereof) capable of mediating editing of a pre-mRNA by a naturally occurring deaminase enzyme. In some cases, such an engineered polynucleotide can be used to affect usage of exons during alternative splicing. The present disclosure describes, in some embodiments, an engineered polynucleotide that can be used to facilitate RNA editing. In some cases, the RNA editing can mediate exon skipping.

The engineered polynucleotide having snRNA sequences and snRNA hairpins (e.g., SmOPT sequences and U7 hairpins or variants thereof) can be circular or can be configured to be circular in a cell. In some embodiments, the engineered polynucleotides are linear. In some embodiments, when the engineered polynucleotides are represented 2-dimensionally, the engineered polynucleotide is linear or circular. The engineered polynucleotides encode for an engineered guide RNA. The engineered guide RNAs may comprise a targeting sequence (or “targeting region” or “targeting domain”) and a recruiting domain (or “recruiting region”). In some embodiments, the engineered guide RNAs are just a targeting sequence.

Small Nuclear RNAs (snRNAs)

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing the first 18 nt of the U7 snRNA (which naturally hybridizes to the spacer element of histone pre-mRNA) with a short targeting (or antisense) sequence of a disease gene, redirects the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this invention.

Wild-type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g. an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g. a pre-mRNA or a mature RNA) at a greater efficiency than a comparable polynucleotide lacking such features.

snRNA Promoters

The compositions and methods of the present disclosure provide engineered polynucleotides encoding for guide RNAs that may also comprise an snRNA promoter. The promoter can promote the transcription of the engineered polynucleotide. The promoter can be operably linked to the engineered polynucleotide. For example, the promoter can be linked to the 5′ or 3′ end of a targeting sequence of the engineered polynucleotide.

A promoter is a non-coding genomic DNA sequence, usually upstream (5′) to the relevant coding sequence, to which RNA polymerase binds before initiating transcription. This binding aligns the RNA polymerase so that transcription will initiate at a specific transcription initiation site. A promoter includes a nucleic acid fragment capable of controlling transcription of another nucleic acid fragment. A promoter is capable of controlling the expression of a coding sequence or functional RNA. Functional RNA includes, but is not limited to, crRNA, tracrRNA, transfer RNA (tRNA) and ribosomal RNA (rRNA). It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters” if the promoters direct RNA synthesis preferably in certain tissues but also in other tissues at reduced levels. Since patterns of expression of a chimeric gene (or genes) introduced into an organism are controlled using promoters, there is an ongoing interest in the isolation of novel promoters which are capable of controlling the expression of a chimeric gene or (genes) at certain levels in specific tissue types or at specific developmental stages.

Certain promoters are able to direct RNA synthesis at relatively similar levels across all tissues. These are called “constitutive promoters” or “tissue-independent” promoters. Constitutive promoters can be divided into strong, moderate and weak according to their effectiveness to direct RNA synthesis. Since it is necessary in many cases to simultaneously express a chimeric gene (or genes) in different tissues to get the desired functions of the gene (or genes), constitutive promoters are especially useful in this consideration.

The promoter sequence can have at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a promoter sequence selected from a group consisting of U1, U2, U3, U4, U5, U6, and U7 promoter sequences. The promoter sequence can be a U1, U2, U3, U4, U5, U6 or U7 promoter sequence.

snRNA Hairpins and Additional Secondary Structures

The compositions and methods of the present disclosure provide engineered polynucleotides encoding for guide RNAs that may include a hairpin or other secondary structures. Hairpins and other secondary structures present in an RNA can increase the stability of an RNA molecule.

As disclosed herein, a hairpin is an RNA duplex wherein a single RNA strand has folded in upon itself to form the RNA duplex. The single RNA strand folds upon itself due to having nucleotide sequences upstream and downstream of the folding region base pairs to each other. A hairpin may have from 10 to 500 nucleotides in length of the entire duplex structure. The stem-loop structure of a hairpin may be from 3 to 15 nucleotides long. A hairpin may be present in any of the engineered guide RNAs disclosed herein. The engineered guide RNAs disclosed herein may have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin may refer to a recruitment hairpin or a hairpin or a non-recruitment hairpin. A hairpin can be located anywhere within the engineered guide RNAs of the present disclosure. In some embodiments, one or more hairpins is present at the 3′ end of an engineered guide RNA of the present disclosure, at the 5′ end of an engineered guide RNA of the present disclosure or within the targeting sequence of the engineered guide RNAs of the present disclosure, or any combination thereof.

A recruitment hairpin, as disclosed herein, may recruit an RNA editing entity, such as ADAR. In some embodiments, a recruitment hairpin is a GluR2 domain or variant thereof. In some embodiments, a recruitment hairpin is an Alu domain or variant thereof.

A non-recruitment hairpin, as disclosed herein, may exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA.

Hairpins and other RNA secondary structures are formed through intermolecular interactions of RNA bases to form base-pairing through Watson-Crick base pairing.

The term “hairpin loop” refers to a single stranded region that loops back on itself and is closed by complementary binding of domains.

The hairpin can comprise a sequence derived from a spliceosomal snRNA, a non-splicesosomal snRNA sequence, or any combination thereof. The hairpin can comprise a sequence that has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a spliceosomal or non-spliceosomal snRNA sequence.

The spliceosomal or non-spliceosomal snRNA sequence can include at least a portion of U1, U2, U4, U5, U6, U7, or any combination thereof. The snRNA sequence can have at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any one of the promoter sequences of SEQ ID NO: 34-SEQ ID NO: 39.

An engineered polynucleotide having snRNA sequences and snRNA hairpins (e.g., SmOPT sequences and U7 hairpins or variants thereof) can comprise a sequence that forms a hairpin secondary structure. In some cases, the sequence can have at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to a sequence of a U7 hairpin.

Other secondary structures can be appended to the engineered polynucleotide. The appended secondary structures can be, but are not limited to, hnRNPA1 and its mutated version, the SIRLOIN nuclear tag, the MALAT1 MG fragment, and Cas9 gRNA containing an additional inert secondary structure.

### Targeting Sequence

The engineered polynucleotide having snRNA sequences and snRNA hairpins (e.g., SmOPT sequences and U7 hairpins or variants thereof) of the present disclosure includes a targeting sequence that directs said construct to the region of interest. The targeting sequence can comprise a sequence of nucleotides that is at least partially complementary to the sequence of interest, thereby providing a means of hybridizing to the target sequence.

Provided herein are polynucleotides and compositions that comprise the same. In an aspect, a polynucleotide can be an engineered polynucleotide. In an embodiment, an engineered polynucleotide can be an engineered polynucleotide. In some embodiments, an engineered polynucleotide of the disclosure may be utilized for RNA editing, for example to prevent or treat a disease or condition. In some cases, an engineered polynucleotide can be used in association with a subject RNA editing entity to edit a target RNA or modulate expression of a polypeptide encoded by the target RNA. In an embodiment, compositions disclosed herein can include engineered polynucleotides capable of facilitating editing by subject RNA editing entities such as ADAR or ADAT polypeptides or biologically active fragments thereof.

Engineered polynucleotides can be engineered in any way suitable for RNA targeting. In an aspect, an engineered polynucleotide generally comprises at least a targeting sequence that allows it to hybridize to a region of a target RNA. In some cases, a targeting sequence may also be referred to as a targeting domain or a targeting region.

In an aspect, a targeting sequence of an engineered polynucleotide allows the engineered polynucleotide to target an RNA sequence through base pairing, such as Watson Crick base pairing. In an embodiment, the targeting sequence can be located at either the N-terminus or C-terminus of the engineered polynucleotide. In some cases, the targeting sequence is located at both termini. The targeting sequence can be of any length. In some cases, the targeting sequence is at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or up to about 200 nucleotides in length. In an embodiment, an engineered polynucleotide comprises a targeting sequence that is about 25-200, 50-150, 75-100, 80-110, 90-120, or 95-115 nucleotides in length. In an embodiment, an engineered polynucleotide comprises a targeting sequence that is about 100 nucleotides in length.

In some cases, a subject targeting sequence comprises at least partial sequence complementarity to a region of a target RNA that at least partially encodes a subject polypeptide. In some cases, a targeting sequence comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence may have a single base mismatch. In other cases, the targeting sequence of a subject engineered polynucleotide comprises at least about 1, 2, 3, 4, 5, 6, 7, 8, 9, 10, 11, 12, 13, 14, 15, 16, 20, 30, 40 or up to about 50 base mismatches. In some aspects, nucleotide mismatches can be associated with structural features provided herein. In some aspects, a targeting sequence comprises at least about 1, 2, 3, 4, 5, 6, 7, 8, 9, 10, 11, 12, 13, 14, or up to about 15 nucleotides that differ in complementarity from a wildtype RNA of a subject target RNA. In some cases, a targeting sequence comprises at least 50 nucleotides having complementarity to a target RNA. In some cases, a targeting sequence comprises from 50 to 150 nucleotides having complementarity to a target RNA. In some cases, a targeting sequence comprises from 50 to 200 nucleotides having complementarity to a target RNA. In some cases, a targeting sequence comprises from 50 to 250 nucleotides having complementarity to a target RNA. In some cases, a targeting sequence comprises from 50 to 300 nucleotides having complementarity to a target RNA. In some cases, a targeting sequence comprises 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 nucleotides having complementarity to a target RNA. In some cases, a targeting sequence comprises more than 50 nucleotides total and has at least 50 nucleotides having complementarity to a target RNA. In some cases, a targeting sequence comprises from 50 to 400 nucleotides total and has from 50 to 150 nucleotides having complementarity to a target RNA. In some cases, a targeting sequence comprises from 50 to 400 nucleotides total and has from 50 to 200 nucleotides having complementarity to a target RNA. In some cases, a targeting sequence comprises from 50 to 400 nucleotides total and has from 50 to 250 nucleotides having complementarity to a target RNA. In some cases, a targeting sequence comprises from 50 to 400 nucleotides total and has from 50 to 300 nucleotides having complementarity to a target RNA. In some cases, the at least 50 nucleotides having complementarity to a target RNA are separated by one or more mismatches, one or more bulges, or one or more loops, or any combination thereof. In some cases, the from 50 to 150 nucleotides having complementarity to a target RNA are separated by one or more mismatches, one or more bulges, or one or more loops, or any combination thereof. In some cases, the from 50 to 200 nucleotides having complementarity to a target RNA are separated by one or more mismatches, one or more bulges, or one or more loops, or any combination thereof. In some cases, the from 50 to 250 nucleotides having complementarity to a target RNA are separated by one or more mismatches, one or more bulges, or one or more loops, or any combination thereof. In some cases, the from 50 to 300 nucleotides having complementarity to a target RNA are separated by one or more mismatches, one or more bulges, or one or more loops, or any combination thereof. For example, a targeting sequence comprises a total of 54 nucleotides wherein, sequentially, 25 nucleotides are complementarity to a target RNA, 4 nucleotides form a bulge, and 25 nucleotides are complementarity to a target RNA. As another example, a targeting sequence comprises a total of 118 nucleotides wherein, sequentially, 25 nucleotides are complementarity to a target RNA, 4 nucleotides form a bulge, 25 nucleotides are complementarity to a target RNA, 14 nucleotides form a loop, and 50 nucleotides are complementary to a target RNA.

In some cases, an engineered polynucleotide of the present disclosure bound to a target RNA forms a double stranded RNA that recruits ADAR and is itself a substrate for ADAR.

In some cases, an engineered polynucleotide can comprise multiple targeting sequences. In some instances, one or more target sequence domains in the engineered polynucleotide can bind to one or more regions of a target RNA. For example, a first targeting sequence can be configured to be at least partially complementary to a first region of a target RNA (e.g. a first exon of a pre-mRNA), while a second targeting sequence can be configured to be at least partially complementary to a second region of a target RNA (e.g. a second exon of a pre-mRNA). In some instances, multiple target sequences can be operatively linked to provide continuous hybridization of multiple regions of a target RNA. In some instances, multiple target sequences can provide non-continuous hybridization of multiple regions of a target RNA. A “non-continuous” overlap or hybridization can refer to hybridization of a first region of a target RNA by a first targeting sequence, along with hybridization of a second region of a target RNA by a second targeting sequence, where the first region and the second region of the target RNA are discontinuous (e.g., where there is intervening sequence between the first and the second region of the target RNA). For example, a targeting sequence can be configured to bind to a portion of a first exon and can comprise a second targeting sequence that is configured to bind to a portion of a second exon, while the intervening sequence between the portion of exon 1 and the portion of exon 2 is not hybridized by either the targeting sequence or the oligo tether. Use of an engineered polynucleotide as described herein configured for non-continuous hybridization can provide a number of benefits. For instance, such a guide can potentially target pre-mRNA during transcription (or shortly thereafter), which can then facilitate chemical modification using a deaminase (e.g. ADAR) co-transcriptionally and thus increase the overall efficiency of the chemical modification. Further, using polynucleotides with non-continuous hybridization while skipping intervening sequence can result in shorter, more specific guide RNA with fewer off-target editing.

In some instances, an engineered polynucleotide configured for non-continuous hybridization to a target RNA can be configured to bind distinct regions or a target RNA separated by intervening sequence. In some instances, the intervening sequence can be at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 bases. In some instances, the targeting sequences can target distinct non-continuous regions of the same intron or exon. In some instances, the targeting sequences can target distinct non-continuous regions of adjacent exons or introns. In some instances, the targeting sequences can target distinct non-continuous regions of distal exons or introns.

### Recruiting Region

In an aspect, a subject engineered polynucleotide comprises an RNA editing entity recruiting domain. An RNA editing entity can be recruited by an RNA editing entity recruiting domain on an engineered polynucleotide. In some cases, a subject engineered polynucleotide is configured to facilitate editing of a base of a nucleotide of a polynucleotide of a region of a subject target RNA, modulation expression of a polypeptide encoded by the subject target RNA, or both. In some cases, an engineered polynucleotide can be configured to facilitate an editing of a base of a nucleotide or polynucleotide of a region of an RNA by a subject RNA editing entity. In order to facilitate editing, an engineered polynucleotide of the disclosure may recruit an RNA editing entity. In certain embodiments, an engineered polynucleotide lacks an RNA editing entity recruiting domain. Either way, a subject engineered polynucleotide can be capable of binding an RNA editing entity, or be bound by it, and facilitate editing of a subject target RNA.

In some examples, a subject targeting sequence comprises an RNA editing entity recruiting domain. An RNA editing entity can be recruited by an RNA editing entity recruiting domain on an engineered polynucleotide. In some examples, a subject engineered polynucleotide is configured to facilitate editing of a base of a nucleotide of a polynucleotide of a region of a subject target RNA, modulation expression of a polypeptide encoded by the subject target RNA, or both. In some cases, an engineered polynucleotide can be configured to facilitate an editing of a base of a nucleotide or polynucleotide of a region of an RNA by a subject RNA editing entity. In order to facilitate editing, an engineered polynucleotide of the disclosure may recruit an RNA editing entity.

Various RNA editing entity recruiting domains can be utilized. In some examples, a recruiting domain comprises: Glutamate ionotropic receptor AMPA type subunit 2 (GluR2), APOBEC, MS2-bacteriophage-coat-protein-recruiting domain, Alu, a TALEN recruiting domain, a Zn-finger polypeptide recruiting domain, a mega-TAL recruiting domain, or a Cas13 recruiting domain, combinations thereof, or modified versions thereof. In some examples, more than one recruiting domain can be included in an engineered polynucleotide of the disclosure. In examples where a recruiting sequence is present, the recruiting sequence can be utilized to position the RNA editing entity to effectively react with a subject target RNA after the targeting sequence (for example, a portion of the targeting sequence that is at least partially complementary to the target RNA), hybridizes to a target RNA. In some cases, a recruiting sequence can allow for transient binding of the RNA editing entity to the engineered polynucleotide. In some examples, the recruiting sequence allows for permanent binding of the RNA editing entity to the engineered polynucleotide. A recruiting sequence can be of any length. In some cases, a recruiting sequence is from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting sequence is no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting sequence is about 45 nucleotides in length. In some cases, at least a portion of a recruiting sequence comprises at least 1 to about 75 nucleotides. In some cases, at least a portion of a recruiting sequence comprises about 45 nucleotides to about 60 nucleotides.

In an embodiment, an RNA editing entity recruiting domain comprises a GluR2 sequence, variant, or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 80%, 85%, 90%, 95%, 98%, 99%, or 100% identity to: GUGGAAUAGUAUAACAAUAUGCUAAAUGUUGUUAUAGUAUCCCAC (SEQ ID NO: 49). In some cases, a recruiting domain can comprise at least about 80% sequence homology to at least about 10, 15, 20, 25, or 30 nucleotides of SEQ ID NO: 49. In some examples, a recruiting domain can comprise at least about 90%, 95%, 96%, 97%, 98%, or 99% sequence homology to SEQ ID NO: 49.

In some examples, a recruiting domain comprises a CRISPR associated recruiting domain sequence. For example, a CRISPR associated recruiting sequence can comprise a Cas protein sequence. In some cases, a Cas13 recruiting domain can comprise a Cas13a recruiting domain, a Cas13b recruiting domain, a Cas13c recruiting domain, or a Cas13d recruiting domain. In some examples, an RNA editing entity recruiting domain can comprise at least about 80% sequence homology to at least about 20 nucleic acids of a Cas13b recruiting domain. In some examples, an RNA editing entity recruiting domain can comprise at least about 80% sequence homology to a Cas13b recruiting domain. In some cases, an RNA editing entity recruiting domain can comprise at least about: 80%, 85%, 90%, or 95% sequence homology to at least about: 15, 20, 25, 30, or 35 nucleic acids of a Cas13b domain. In some examples, at least a portion of an RNA editing entity recruiting domain can comprise at least about 80% sequence homology to a Cas13b domain encoding sequence. In some examples, at least a portion of an RNA editing entity recruiting domain can comprise at least about 85% sequence homology to a Cas13b domain encoding sequence. In some examples, at least a portion of an RNA editing entity recruiting domain can comprise at least about 90% sequence homology to a Cas13b domain encoding sequence. In some examples, at least a portion of an RNA editing entity recruiting domain can comprise at least about 95% sequence homology to a Cas13b domain encoding sequence. In some examples, a Cas13b-domain-encoding sequence can be a non-naturally occurring sequence. In some examples, a Cas13b-domain-encoding sequence can comprise a modified portion. In some examples, a Cas13b-domain-encoding sequence can comprise a portion of a naturally occurring Cas13b-domain-encoding-sequence.

Any number of recruiting sequences may be found in an engineered polynucleotide of the present disclosure. In some examples, at least about 1, 2, 3, 4, 5, 6, 7, 8, 9, or up to about 10 recruiting sequences are included in an engineered polynucleotide. Recruiting sequences may be located at any position of subject polynucleotides. In some cases, a recruiting sequence is on an N-terminus, middle, or C-terminus of a polynucleotide. A recruiting sequence can be upstream or downstream of a targeting sequence. In some cases, a recruiting sequence flanks a targeting sequence of a subject polynucleotide. A recruiting sequence can comprise all ribonucleotides or deoxyribonucleotides, although a recruiting sequence comprising both ribo- and deoxyribonucleotides is not excluded.

In some examples, the engineered polynucleotides disclosed herein lack a recruiting region and recruitment of the RNA editing entity is effectuated by the double stranded substrate formed by the engineered polynucleotide and the target RNA. In some examples, an engineered polynucleotide disclosed herein, when present in an aqueous solution and not bound to the target RNA molecule, does not recruit an RNA editing entity. In some examples, when present in an aqueous solution and not bound to the target RNA molecule, if it binds to the RNA editing entity, an engineered polynucleotide disclosed herein does so with a dissociation constant of about greater than or equal to 500 nM. In some examples, the dissociation constant is about 22 nM. In some examples, the engineered polynucleotides disclosed herein, when present in an aqueous solution and not bound to the target RNA molecule, lack a structural feature. In some examples, the engineered polynucleotides disclosed herein, when present in an aqueous solution and not bound to the target RNA molecule, lack a bulge, an internal loop, a hairpin, or any combination thereof. In some examples, the engineered polynucleotides disclosed herein, when present in an aqueous solution and not bound to the target RNA molecule, are linear and do not comprise any structural features.

In cases where a recruiting sequence is absent, an engineered polynucleotide is still capable of associating with a subject RNA editing entity (e.g., ADAR) to facilitate editing of a target RNA and/or modulate expression of a polypeptide encoded by a subject target RNA. This may be achieved through structural features. Structural features may comprise any one of a: mismatch, symmetrical bulge, asymmetrical bulge, symmetrical internal loop, asymmetrical internal loop, hairpins, wobble base pairs, a structured motif, circularized RNA, chemical modification, or any combination thereof. In an aspect, a double stranded RNA (dsRNA) substrate, for example hybridized polynucleotide strands, can be formed upon hybridization of an engineered polynucleotide of the present disclosure to a target RNA. Described herein is a feature, which corresponds to one of several structural features that may be present in a dsRNA substrate of the present disclosure. Examples of features include a mismatch, a bulge (symmetrical bulge or asymmetrical bulge), an internal loop (symmetrical internal loop or asymmetrical internal loop), or a hairpin (a recruiting hairpin or a hairpin comprising a non-targeting domain). Engineered polynucleotides of the present disclosure may have from 1 to 50 features. Engineered polynucleotides of the present disclosure may have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features.

As disclosed herein, a structured motif comprises two or more features in a dsRNA substrate.

A double stranded RNA (dsRNA) substrate is formed upon hybridization of an engineered guide RNA of the present disclosure to a target RNA. As disclosed herein, a mismatch refers to a nucleotide in a guide RNA that is unpaired to an opposing nucleotide in a target RNA within the dsRNA. A mismatch can comprise any two nucleotides that do not base pair, are not complementary, or both. In some embodiments, a mismatch is an A/C mismatch. An A/C mismatch may comprise a C in an engineered guide RNA of the present disclosure opposite an A in a target RNA. An A/C mismatch may comprise a A in an engineered guide RNA of the present disclosure opposite an C in a target RNA. In an embodiment, a G/G mismatch may comprise a Gin an engineered guide RNA of the present disclosure opposite a Gin a target RNA. In some embodiments, a mismatch positioned 5′ of the edit site may facilitate base-flipping of the target A to be edited. A mismatch may also help confer sequence specificity. In an embodiment, a mismatch comprises a G/G mismatch. In an embodiment, a mismatch comprises an A/C mismatch, wherein the A is in the target RNA and the C is in the targeting sequence of the engineered polynucleotide. In another embodiment, the A in the A/C mismatch is the base of the nucleotide in the target RNA edited by a subject RNA editing entity.

A mismatch as described herein can be located at a distance proximal to either end of a targeting sequence. In some instances, a mismatch can be located at a distance of from about 1 base to about 200 bases, from about 2 bases to about 200 bases, from about 3 bases to about 200 bases, from about 4 bases to about 200 bases, from about 5 bases to about 200 bases, from about 6 bases to about 200 bases, from about 7 bases to about 200 bases, from about 8 bases to about 200 bases, from about 9 bases to about 200 bases, from about 10 bases to about 200 bases, from about 11 bases to about 200 bases, from about 12 bases to about 200 bases, from about 13 bases to about 200 bases, from about 14 bases to about 200 bases, from about 15 bases to about 200 bases, from about 16 bases to about 200 bases, from about 17 bases to about 200 bases, from about 18 bases to about 200 bases, from about 19 bases to about 200 bases, from about 20 bases to about 200 bases, from about 21 bases to about 200 bases, from about 22 bases to about 200 bases, from about 23 bases to about 200 bases, from about 24 bases to about 200 bases, from about 25 bases to about 200 bases, from about 26 bases to about 200 bases, from about 27 bases to about 200 bases, from about 28 bases to about 200 bases, from about 29 bases to about 200 bases, from about 30 bases to about 200 bases, from about 31 bases to about 200 bases, from about 32 bases to about 200 bases, from about 33 bases to about 200 bases, from about 34 bases to about 200 bases, from about 35 bases to about 200 bases, from about 36 bases to about 200 bases, from about 37 bases to about 200 bases, from about 38 bases to about 200 bases, from about 39 bases to about 200 bases, from about 40 bases to about 200 bases, from about 41 bases to about 200 bases, from about 42 bases to about 200 bases, from about 43 bases to about 200 bases, from about 44 bases to about 200 bases, from about 45 bases to about 200 bases, from about 46 bases to about 200 bases, from about 47 bases to about 200 bases, from about 48 bases to about 200 bases, from about 49 bases to about 200 bases, from about 50 bases to about 200 bases, from about 51 bases to about 200 bases, from about 52 bases to about 200 bases, from about 53 bases to about 200 bases, from about 54 bases to about 200 bases, from about 55 bases to about 200 bases, from about 56 bases to about 200 bases, from about 57 bases to about 200 bases, from about 58 bases to about 200 bases, from about 59 bases to about 200 bases, from about 60 bases to about 200 bases, from about 61 bases to about 200 bases, from about 62 bases to about 200 bases, from about 63 bases to about 200 bases, from about 64 bases to about 200 bases, from about 65 bases to about 200 bases, from about 66 bases to about 200 bases, from about 67 bases to about 200 bases, from about 68 bases to about 200 bases, from about 69 bases to about 200 bases, from about 70 bases to about 200 bases, from about 71 bases to about 200 bases, from about 72 bases to about 200 bases, from about 73 bases to about 200 bases, from about 74 bases to about 200 bases, from about 75 bases to about 200 bases, from about 76 bases to about 200 bases, from about 77 bases to about 200 bases, from about 78 bases to about 200 bases, from about 79 bases to about 200 bases, from about 80 bases to about 200 bases, from about 81 bases to about 200 bases, from about 82 bases to about 200 bases, from about 83 bases to about 200 bases, from about 84 bases to about 200 bases, from about 85 bases to about 200 bases, from about 86 bases to about 200 bases, from about 87 bases to about 200 bases, from about 88 bases to about 200 bases, from about 89 bases to about 200 bases, from about 90 bases to about 200 bases, from about 91 bases to about 200 bases, from about 92 bases to about 200 bases, from about 93 bases to about 200 bases, from about 94 bases to about 200 bases, from about 95 bases to about 200 bases, from about 96 bases to about 200 bases, from about 97 bases to about 200 bases, from about 98 bases to about 200 bases, from about 99 bases to about 200 bases, from about 100 bases to about 200 bases, from about 101 bases to about 200 bases, from about 102 bases to about 200 bases, from about 103 bases to about 200 bases, from about 104 bases to about 200 bases, from about 105 bases to about 200 bases, from about 106 bases to about 200 bases, from about 107 bases to about 200 bases, from about 108 bases to about 200 bases, from about 109 bases to about 200 bases, from about 110 bases to about 200 bases, from about 111 bases to about 200 bases, from about 112 bases to about 200 bases, from about 113 bases to about 200 bases, from about 114 bases to about 200 bases, from about 115 bases to about 200 bases, from about 116 bases to about 200 bases, from about 117 bases to about 200 bases, from about 118 bases to about 200 bases, from about 119 bases to about 200 bases, from about 120 bases to about 200 bases, from about 121 bases to about 200 bases, from about 122 bases to about 200 bases, from about 123 bases to about 200 bases, from about 124 bases to about 200 bases, from about 125 bases to about 200 bases, from about 126 bases to about 200 bases, from about 127 bases to about 200 bases, from about 128 bases to about 200 bases, from about 129 bases to about 200 bases, from about 130 bases to about 200 bases, from about 131 bases to about 200 bases, from about 132 bases to about 200 bases, from about 133 bases to about 200 bases, from about 134 bases to about 200 bases, from about 135 bases to about 200 bases, from about 136 bases to about 200 bases, from about 137 bases to about 200 bases, from about 138 bases to about 200 bases, from about 139 bases to about 200 bases, from about 140 bases to about 200 bases, from about 141 bases to about 200 bases, from about 142 bases to about 200 bases, from about 143 bases to about 200 bases, from about 144 bases to about 200 bases, from about 145 bases to about 200 bases, from about 146 bases to about 200 bases, from about 147 bases to about 200 bases, from about 148 bases to about 200 bases, from about 149 bases to about 200 bases, from about 150 bases to about 200 bases, from about 151 bases to about 200 bases, from about 152 bases to about 200 bases, from about 153 bases to about 200 bases, from about 154 bases to about 200 bases, from about 155 bases to about 200 bases, from about 156 bases to about 200 bases, from about 157 bases to about 200 bases, from about 158 bases to about 200 bases, from about 159 bases to about 200 bases, from about 160 bases to about 200 bases, from about 161 bases to about 200 bases, from about 162 bases to about 200 bases, from about 163 bases to about 200 bases, from about 164 bases to about 200 bases, from about 165 bases to about 200 bases, from about 166 bases to about 200 bases, from about 167 bases to about 200 bases, from about 168 bases to about 200 bases, from about 169 bases to about 200 bases, from about 170 bases to about 200 bases, from about 171 bases to about 200 bases, from about 172 bases to about 200 bases, from about 173 bases to about 200 bases, from about 174 bases to about 200 bases, from about 175 bases to about 200 bases, from about 176 bases to about 200 bases, from about 177 bases to about 200 bases, from about 178 bases to about 200 bases, from about 179 bases to about 200 bases, from about 180 bases to about 200 bases, from about 181 bases to about 200 bases, from about 182 bases to about 200 bases, from about 183 bases to about 200 bases, from about 184 bases to about 200 bases, from about 185 bases to about 200 bases, from about 186 bases to about 200 bases, from about 187 bases to about 200 bases, from about 188 bases to about 200 bases, from about 189 bases to about 200 bases, from about 190 bases to about 200 bases, from about 191 bases to about 200 bases, from about 192 bases to about 200 bases, from about 193 bases to about 200 bases, from about 194 bases to about 200 bases, from about 195 bases to about 200 bases, from about 196 bases to about 200 bases, from about 197 bases to about 200 bases, from about 198 bases to about 200 bases, or from about 199 bases to about 200 bases, with respect to either end of a targeting sequence. In some instances, a mismatch can be located at a distance of at least 1 base, at least 2 bases, at least 3 bases, at least 4 bases, at least 5 bases, at least 6 bases, at least 7 bases, at least 8 bases, at least 9 bases, at least 10 bases, at least 11 bases, at least 12 bases, at least 13 bases, at least 14 bases, at least 15 bases, at least 16 bases, at least 17 bases, at least 18 bases, at least 19 bases, at least 20 bases, at least 21 bases, at least 22 bases, at least 23 bases, at least 24 bases, at least 25 bases, at least 26 bases, at least 27 bases, at least 28 bases, at least 29 bases, at least 30 bases, at least 31 bases, at least 32 bases, at least 33 bases, at least 34 bases, at least 35 bases, at least 36 bases, at least 37 bases, at least 38 bases, at least 39 bases, at least 40 bases, at least 41 bases, at least 42 bases, at least 43 bases, at least 44 bases, at least 45 bases, at least 46 bases, at least 47 bases, at least 48 bases, at least 49 bases, at least 50 bases, at least 51 bases, at least 52 bases, at least 53 bases, at least 54 bases, at least 55 bases, at least 56 bases, at least 57 bases, at least 58 bases, at least 59 bases, at least 60 bases, at least 61 bases, at least 62 bases, at least 63 bases, at least 64 bases, at least 65 bases, at least 66 bases, at least 67 bases, at least 68 bases, at least 69 bases, at least 70 bases, at least 71 bases, at least 72 bases, at least 73 bases, at least 74 bases, at least 75 bases, at least 76 bases, at least 77 bases, at least 78 bases, at least 79 bases, at least 80 bases, at least 81 bases, at least 82 bases, at least 83 bases, at least 84 bases, at least 85 bases, at least 86 bases, at least 87 bases, at least 88 bases, at least 89 bases, at least 90 bases, at least 91 bases, at least 92 bases, at least 93 bases, at least 94 bases, at least 95 bases, at least 96 bases, at least 97 bases, at least 98 bases, at least 99 bases, at least 100 bases, at least 101 bases, at least 102 bases, at least 103 bases, at least 104 bases, at least 105 bases, at least 106 bases, at least 107 bases, at least 108 bases, at least 109 bases, at least 110 bases, at least 111 bases, at least 112 bases, at least 113 bases, at least 114 bases, at least 115 bases, at least 116 bases, at least 117 bases, at least 118 bases, at least 119 bases, at least 120 bases, at least 121 bases, at least 122 bases, at least 123 bases, at least 124 bases, at least 125 bases, at least 126 bases, at least 127 bases, at least 128 bases, at least 129 bases, at least 130 bases, at least 131 bases, at least 132 bases, at least 133 bases, at least 134 bases, at least 135 bases, at least 136 bases, at least 137 bases, at least 138 bases, at least 139 bases, at least 140 bases, at least 141 bases, at least 142 bases, at least 143 bases, at least 144 bases, at least 145 bases, at least 146 bases, at least 147 bases, at least 148 bases, at least 149 bases, at least 150 bases, at least 151 bases, at least 152 bases, at least 153 bases, at least 154 bases, at least 155 bases, at least 156 bases, at least 157 bases, at least 158 bases, at least 159 bases, at least 160 bases, at least 161 bases, at least 162 bases, at least 163 bases, at least 164 bases, at least 165 bases, at least 166 bases, at least 167 bases, at least 168 bases, at least 169 bases, at least 170 bases, at least 171 bases, at least 172 bases, at least 173 bases, at least 174 bases, at least 175 bases, at least 176 bases, at least 177 bases, at least 178 bases, at least 179 bases, at least 180 bases, at least 181 bases, at least 182 bases, at least 183 bases, at least 184 bases, at least 185 bases, at least 186 bases, at least 187 bases, at least 188 bases, at least 189 bases, at least 190 bases, at least 191 bases, at least 192 bases, at least 193 bases, at least 194 bases, at least 195 bases, at least 196 bases, at least 197 bases, at least 198 bases, at least 199 bases, or at least 200 bases from either end of a targeting sequence.

In an aspect, a structural feature can form in an engineered polynucleotide independently. In other cases, a structural feature can form when an engineered polynucleotide binds to a target RNA. A structural feature can also form when an engineered polynucleotide associates with other molecules such as a peptide, a nucleotide, or a small molecule. In certain embodiments, a structural feature of an engineered polynucleotide can be formed independent of a target RNA, and its structure can change as a result of the engineered polypeptide hybridization with a target RNA region. In certain embodiments, a structural feature is present when an engineered polynucleotide is in association with a target RNA.

In some cases, a structural feature is a hairpin. In some cases, an engineered polynucleotide can lack a hairpin domain. In other cases, an engineered polynucleotide can contain a hairpin domain or more than one hairpin domain. A hairpin can be located anywhere in a polynucleotide. As disclosed herein, a hairpin is an RNA duplex wherein a single RNA strand has folded in upon itself to form the RNA duplex. The single RNA strand folds upon itself due to having nucleotide sequences upstream and downstream of the folding region base pairs to each other. A hairpin may have from 10 to 500 nucleotides in length of the entire duplex structure. The stem-loop structure of a hairpin may be from 3 to 15 nucleotides long. A hairpin may be present in any of the engineered polynucleotides disclosed herein. The engineered polynucleotides disclosed herein may have from 1 to 10 hairpins. In some embodiments, the engineered polynucleotides disclosed herein have 1 hairpin. In some embodiments, the engineered polynucleotides disclosed herein have 2 hairpins. As disclosed herein, a hairpin may refer to a recruitment hairpin or a hairpin or a non-recruitment hairpin. A hairpin can be located anywhere within the engineered polynucleotides of the present disclosure. In some embodiments, one or more hairpins is present at the 3′ end of an engineered polynucleotide of the present disclosure, at the 5′ end of an engineered polynucleotide of the present disclosure or within the targeting sequence of an engineered polynucleotide of the present disclosure, or any combination thereof.

In one aspect, a structural feature comprises a recruitment hairpin, as disclosed herein. A recruitment hairpin may recruit an RNA editing entity, such as ADAR. In some embodiments, a recruitment hairpin comprises a GluR2 domain or variant thereof. In some embodiments, a recruitment hairpin comprises an Alu domain or variant thereof.

In yet another aspect, a structural feature comprises a non-recruitment hairpin. A non-recruitment hairpin, as disclosed herein, may exhibit functionality that improves localization of the engineered polynucleotide to the target RNA. In some embodiments, the non-recruitment hairpin improves nuclear retention. In some embodiments, the non-recruitment hairpin comprises a hairpin from U7 snRNA.

In another aspect, a structural feature comprises a wobble base. A wobble base pair refers to two bases that weakly pair. For example, a wobble base pair of the present disclosure may refer to a G paired with a U.

A hairpin of the present disclosure can be of any length. In an aspect, a hairpin can be from about 5-200 or more nucleotides. In some cases, a hairpin can comprise about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 or more nucleotides. In other cases, a hairpin can also comprise 5 to 10, 5 to 20, 5 to 30, 5 to 40, 5 to 50, 5 to 60, 5 to 70, 5 to 80, 5 to 90, 5 to 100, 5 to 110, 5 to 120, 5 to 130, 5 to 140, 5 to 150, 5 to 160, 5 to 170, 5 to 180, 5 to 190, 5 to 200, 5 to 210, 5 to 220, 5 to 230, 5 to 240, 5 to 250, 5 to 260, 5 to 270, 5 to 280, 5 to 290, 5 to 300, 5 to 310, 5 to 320, 5 to 330, 5 to 340, 5 to 350, 5 to 360, 5 to 370, 5 to 380, 5 to 390, or 5 to 400 nucleotides. A hairpin is a structural feature formed from a single strand of RNA with sufficient complementarity to itself to hybridize into a double stranded RNA motif/structure consisting of double-stranded hybridized RNA separated by a nucleotide loop.

In some cases, a structural feature is a bulge. A bulge can comprise a single (intentional) nucleic acid mismatch between the target strand and an engineered polynucleotide strand. In other cases, more than one consecutive mismatch between strands constitutes a bulge as long as the bulge region, mismatched stretch of bases, is flanked on both sides with hybridized, complementary dsRNA regions. A bulge can be located at any location of a polynucleotide. In some cases, a bulge is located from about 30 to about 70 nucleotides from a 5′ hydroxyl or the 3′ hydroxyl.

In an embodiment, a double stranded RNA (dsRNA) substrate is formed upon hybridization of an engineered polynucleotide of the present disclosure to a target RNA. As disclosed herein, a bulge refers to the structure formed upon formation of the dsRNA substrate, where nucleotides in either the engineered polynucleotide or the target RNA are not complementary to their positional counterparts on the opposite strand. A bulge may change the secondary or tertiary structure of the dsRNA substrate. A bulge may have from 1 to 4 nucleotides on the engineered polynucleotide side of the dsRNA substrate or the target RNA side of the dsRNA substrate. In some embodiments, the engineered polynucleotides of the present disclosure have 2 bulges. In some embodiments, the engineered polynucleotides of the present disclosure have 3 bulges. In some embodiments, the engineered polynucleotides of the present disclosure have 4 bulges. In some embodiments, the presence of a bulge in a dsRNA substrate may position ADAR to selectively edit the target A in the target RNA and reduce off-target editing of non-targets. In some embodiments, the presence of a bulge in a dsRNA substrate may recruit additional ADAR. Bulges in dsRNA substrates disclosed herein may recruit other proteins, such as other RNA editing entities. In some embodiments, a bulge positioned 5′ of the edit site may facilitate base-flipping of the target A to be edited. A bulge may also help confer sequence specificity. A bulge may help direct ADAR editing by constraining it in an orientation that yield selective editing of the target A.

In an aspect, a double stranded RNA (dsRNA) substrate is formed upon hybridization of an engineered polynucleotide of the present disclosure to a target RNA. A bulge may be a symmetrical bulge or an asymmetrical bulge. A bulge may be formed by 1 to 4 participating nucleotides on either the guide RNA side or the target RNA side of the dsRNA substrate. A symmetrical bulge is formed when the same number of nucleotides is present on each side of the bulge. A symmetrical bulge may have from 2 to 4 nucleotides on the engineered polynucleotide side of the dsRNA substrate or the target RNA side of the dsRNA substrate. For example, a symmetrical bulge in a dsRNA substrate of the present disclosure may have the same number of nucleotides on the engineered polynucleotide side and the target RNA side of the dsRNA substrate. A symmetrical bulge of the present disclosure may be formed by 2 nucleotides on the engineered polynucleotide side of the dsRNA target and 2 nucleotides on the target RNA side of the dsRNA substrate. A symmetrical bulge of the present disclosure may be formed by 3 nucleotides on the engineered polynucleotide side of the dsRNA target and 3 nucleotides on the target RNA side of the dsRNA substrate. A symmetrical bulge of the present disclosure may be formed by 4 nucleotides on the engineered polynucleotide side of the dsRNA target and 4 nucleotides on the target RNA side of the dsRNA substrate.

A double stranded RNA (dsRNA) substrate is formed upon hybridization of an engineered guide RNA of the present disclosure to a target RNA. A bulge may be a symmetrical bulge or an asymmetrical bulge. An asymmetrical bulge is formed when a different number of nucleotides is present on each side of the bulge. An asymmetrical bulge may have from 1 to 4 participating nucleotides on either the guide RNA side or the target RNA side of the dsRNA substrate. For example, an asymmetrical bulge in a dsRNA substrate of the present disclosure may have different numbers of nucleotides on the engineered guide RNA side and the target RNA side of the dsRNA substrate. An asymmetrical bulge of the present disclosure may be formed by 0 nucleotides on the engineered guide RNA side of the dsRNA substrate and 1 nucleotide on the target RNA side of the dsRNA substrate. An asymmetrical bulge of the present disclosure may be formed by 0 nucleotides on the target RNA side of the dsRNA substrate and 1 nucleotide on the engineered guide RNA side of the dsRNA substrate. An asymmetrical bulge of the present disclosure may be formed by 0 nucleotides on the engineered guide RNA side of the dsRNA substrate and 2 nucleotides on the target RNA side of the dsRNA substrate. An asymmetrical bulge of the present disclosure may be formed by 0 nucleotides on the target RNA side of the dsRNA substrate and 2 nucleotides on the engineered guide RNA side of the dsRNA substrate. An asymmetrical bulge of the present disclosure may be formed by 0 nucleotides on the engineered guide RNA side of the dsRNA substrate and 3 nucleotides on the target RNA side of the dsRNA substrate. An asymmetrical bulge of the present disclosure may be formed by 0 nucleotides on the target RNA side of the dsRNA substrate and 3 nucleotides on the engineered guide RNA side of the dsRNA substrate. An asymmetrical bulge of the present disclosure may be formed by 0 nucleotides on the engineered guide RNA side of the dsRNA substrate and 4 nucleotides on the target RNA side of the dsRNA substrate. An asymmetrical bulge of the present disclosure may be formed by 0 nucleotides on the target RNA side of the dsRNA substrate and 4 nucleotides on the engineered guide RNA side of the dsRNA substrate. An asymmetrical bulge of the present disclosure may be formed by 1 nucleotide on the engineered guide RNA side of the dsRNA substrate and 2 nucleotides on the target RNA side of the dsRNA substrate. An asymmetrical bulge of the present disclosure may be formed by 1 nucleotide on the target RNA side of the dsRNA substrate and 2 nucleotides on the engineered guide RNA side of the dsRNA substrate. An asymmetrical bulge of the present disclosure may be formed by 1 nucleotide on the engineered guide RNA side of the dsRNA substrate and 3 nucleotides on the target RNA side of the dsRNA substrate. An asymmetrical bulge of the present disclosure may be formed by 1 nucleotide on the target RNA side of the dsRNA substrate and 3 nucleotides on the engineered guide RNA side of the dsRNA substrate. An asymmetrical bulge of the present disclosure may be formed by 1 nucleotide on the engineered guide RNA side of the dsRNA substrate and 4 nucleotides on the target RNA side of the dsRNA substrate. An asymmetrical bulge of the present disclosure may be formed by 1 nucleotide on the target RNA side of the dsRNA substrate and 4 nucleotides on the engineered guide RNA side of the dsRNA substrate. An asymmetrical bulge of the present disclosure may be formed by 2 nucleotides on the engineered guide RNA side of the dsRNA substrate and 3 nucleotides on the target RNA side of the dsRNA substrate. An asymmetrical bulge of the present disclosure may be formed by 2 nucleotides on the target RNA side of the dsRNA substrate and 3 nucleotides on the engineered guide RNA side of the dsRNA substrate. An asymmetrical bulge of the present disclosure may be formed by 2 nucleotides on the engineered guide RNA side of the dsRNA substrate and 4 nucleotides on the target RNA side of the dsRNA substrate. An asymmetrical bulge of the present disclosure may be formed by 2 nucleotides on the target RNA side of the dsRNA substrate and 4 nucleotides on the engineered guide RNA side of the dsRNA substrate. An asymmetrical bulge of the present disclosure may be formed by 3 nucleotides on the engineered guide RNA side of the dsRNA substrate and 4 nucleotides on the target RNA side of the dsRNA substrate. An asymmetrical bulge of the present disclosure may be formed by 3 nucleotides on the target RNA side of the dsRNA substrate and 4 nucleotides on the engineered guide RNA side of the dsRNA substrate.

In an aspect, a double stranded RNA (dsRNA) substrate is formed upon hybridization of an engineered polynucleotide of the present disclosure to a target RNA. As disclosed herein, an internal loop refers to the structure formed upon formation of the dsRNA substrate, where nucleotides in either the engineered polynucleotide or the target RNA are not complementary to their positional counterparts on the opposite strand and where one side of the internal loop, either on the target RNA side or the engineered polynucleotide side of the dsRNA substrate, has greater than 5 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. One side of the internal loop, either on the target RNA side or the engineered polynucleotide side of the dsRNA substrate, may be formed by from 5 to 150 nucleotides. One side of the internal loop may be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop may be formed by 5 nucleotides. One side of the internal loop may be formed by 10 nucleotides. One side of the internal loop may be formed by 15 nucleotides. One side of the internal loop may be formed by 20 nucleotides. One side of the internal loop may be formed by 25 nucleotides. One side of the internal loop may be formed by 30 nucleotides. One side of the internal loop may be formed by 35 nucleotides. One side of the internal loop may be formed by 40 nucleotides. One side of the internal loop may be formed by 45 nucleotides. One side of the internal loop may be formed by 50 nucleotides. One side of the internal loop may be formed by 55 nucleotides. One side of the internal loop may be formed by 60 nucleotides. One side of the internal loop may be formed by 65 nucleotides. One side of the internal loop may be formed by 70 nucleotides. One side of the internal loop may be formed by 75 nucleotides. One side of the internal loop may be formed by 80 nucleotides. One side of the internal loop may be formed by 85 nucleotides. One side of the internal loop may be formed by 90 nucleotides. One side of the internal loop may be formed by 95 nucleotides. One side of the internal loop may be formed by 100 nucleotides. One side of the internal loop may be formed by 110 nucleotides. One side of the internal loop may be formed by 120 nucleotides. One side of the internal loop may be formed by 130 nucleotides. One side of the internal loop may be formed by 140 nucleotides. One side of the internal loop may be formed by 150 nucleotides. One side of the internal loop may be formed by 200 nucleotides. One side of the internal loop may be formed by 250 nucleotides. One side of the internal loop may be formed by 300 nucleotides. One side of the internal loop may be formed by 350 nucleotides. One side of the internal loop may be formed by 400 nucleotides. One side of the internal loop may be formed by 450 nucleotides. One side of the internal loop may be formed by 500 nucleotides. One side of the internal loop may be formed by 600 nucleotides. One side of the internal loop may be formed by 700 nucleotides. One side of the internal loop may be formed by 800 nucleotides. One side of the internal loop may be formed by 900 nucleotides. One side of the internal loop may be formed by 1000 nucleotides. An internal loop may be a symmetrical internal loop or an asymmetrical internal loop. Internal loops present in the vicinity of the edit site may help with base flipping of the target A in the target RNA to be edited. A double stranded RNA (dsRNA) substrate is formed upon hybridization of an engineered polynucleotide of the present disclosure to a target RNA. An internal loop may be a symmetrical internal loop or an asymmetrical internal loop. A symmetrical internal loop is formed when the same number of nucleotides is present on each side of the internal loop. For example, a symmetrical internal loop in a dsRNA substrate of the present disclosure may have the same number of nucleotides on the engineered polynucleotide side and the target RNA side of the dsRNA substrate. A symmetrical internal loop of the present disclosure may be formed by from 5 to 150 nucleotides on the engineered polynucleotide side of the dsRNA target and from 5 to 150 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by from 5 to 1000 nucleotides on the engineered polynucleotide side of the dsRNA target and from 5 to 1000 nucleotides on the target RNA side of the dsRNA substrate, wherein the number of nucleotides is the same on the engineered side of the dsRNA target and the target RNA side of the dsRNA substrate. A symmetrical internal loop of the present disclosure may be formed by 5 nucleotides on the engineered polynucleotide side of the dsRNA target and 5 nucleotides on the target RNA side of the dsRNA substrate. A symmetrical internal loop of the present disclosure may be formed by 6 nucleotides on the engineered polynucleotide side of the dsRNA target and 6 nucleotides on the target RNA side of the dsRNA substrate. A symmetrical internal loop of the present disclosure may be formed by 7 nucleotides on the engineered polynucleotide side of the dsRNA target and 7 nucleotides on the target RNA side of the dsRNA substrate. A symmetrical internal loop of the present disclosure may be formed by 8 nucleotides on the engineered polynucleotide side of the dsRNA target and 8 nucleotides on the target RNA side of the dsRNA substrate. A symmetrical internal loop of the present disclosure may be formed by 9 nucleotides on the engineered polynucleotide side of the dsRNA target and 9 nucleotides on the target RNA side of the dsRNA substrate. A symmetrical internal loop of the present disclosure may be formed by 10 nucleotides on the engineered polynucleotide side of the dsRNA target and 10 nucleotides on the target RNA side of the dsRNA substrate. A symmetrical internal loop of the present disclosure may be formed by 15 nucleotides on the engineered polynucleotide side of the dsRNA target and 15 nucleotides on the target RNA side of the dsRNA substrate. A symmetrical internal loop of the present disclosure may be formed by 20 nucleotides on the engineered polynucleotide side of the dsRNA target and 20 nucleotides on the target RNA side of the dsRNA substrate. A symmetrical internal loop of the present disclosure may be formed by 30 nucleotides on the engineered polynucleotide side of the dsRNA target and 30 nucleotides on the target RNA side of the dsRNA substrate. A symmetrical internal loop of the present disclosure may be formed by 40 nucleotides on the engineered polynucleotide side of the dsRNA target and 40 nucleotides on the target RNA side of the dsRNA substrate. A symmetrical internal loop of the present disclosure may be formed by 50 nucleotides on the engineered polynucleotide side of the dsRNA target and 50 nucleotides on the target RNA side of the dsRNA substrate. A symmetrical internal loop of the present disclosure may be formed by 60 nucleotides on the engineered polynucleotide side of the dsRNA target and 60 nucleotides on the target RNA side of the dsRNA substrate. A symmetrical internal loop of the present disclosure may be formed by 70 nucleotides on the engineered polynucleotide side of the dsRNA target and 70 nucleotides on the target RNA side of the dsRNA substrate. A symmetrical internal loop of the present disclosure may be formed by 80 nucleotides on the engineered polynucleotide side of the dsRNA target and 80 nucleotides on the target RNA side of the dsRNA substrate. A symmetrical internal loop of the present disclosure may be formed by 90 nucleotides on the engineered polynucleotide side of the dsRNA target and 90 nucleotides on the target RNA side of the dsRNA substrate. A symmetrical internal loop of the present disclosure may be formed by 100 nucleotides on the engineered polynucleotide side of the dsRNA target and 100 nucleotides on the target RNA side of the dsRNA substrate. A symmetrical internal loop of the present disclosure may be formed by 110 nucleotides on the engineered polynucleotide side of the dsRNA target and 110 nucleotides on the target RNA side of the dsRNA substrate. A symmetrical internal loop of the present disclosure may be formed by 120 nucleotides on the engineered polynucleotide side of the dsRNA target and 120 nucleotides on the target RNA side of the dsRNA substrate. A symmetrical internal loop of the present disclosure may be formed by 130 nucleotides on the engineered polynucleotide side of the dsRNA target and 130 nucleotides on the target RNA side of the dsRNA substrate. A symmetrical internal loop of the present disclosure may be formed by 140 nucleotides on the engineered polynucleotide side of the dsRNA target and 140 nucleotides on the target RNA side of the dsRNA substrate. A symmetrical internal loop of the present disclosure may be formed by 150 nucleotides on the engineered polynucleotide side of the dsRNA target and 150 nucleotides on the target RNA side of the dsRNA substrate. A symmetrical internal loop of the present disclosure may be formed by 200 nucleotides on the engineered polynucleotide side of the dsRNA target and 200 nucleotides on the target RNA side of the dsRNA substrate. A symmetrical internal loop of the present disclosure may be formed by 250 nucleotides on the engineered polynucleotide side of the dsRNA target and 250 nucleotides on the target RNA side of the dsRNA substrate. A symmetrical internal loop of the present disclosure may be formed by 300 nucleotides on the engineered polynucleotide side of the dsRNA target and 300 nucleotides on the target RNA side of the dsRNA substrate. A symmetrical internal loop of the present disclosure may be formed by 350 nucleotides on the engineered polynucleotide side of the dsRNA target and 350 nucleotides on the target RNA side of the dsRNA substrate. A symmetrical internal loop of the present disclosure may be formed by 400 nucleotides on the engineered polynucleotide side of the dsRNA target and 400 nucleotides on the target RNA side of the dsRNA substrate. A symmetrical internal loop of the present disclosure may be formed by 450 nucleotides on the engineered polynucleotide side of the dsRNA target and 450 nucleotides on the target RNA side of the dsRNA substrate. A symmetrical internal loop of the present disclosure may be formed by 500 nucleotides on the engineered polynucleotide side of the dsRNA target and 500 nucleotides on the target RNA side of the dsRNA substrate. A symmetrical internal loop of the present disclosure may be formed by 600 nucleotides on the engineered polynucleotide side of the dsRNA target and 600 nucleotides on the target RNA side of the dsRNA substrate. A symmetrical internal loop of the present disclosure may be formed by 700 nucleotides on the engineered polynucleotide side of the dsRNA target and 700 nucleotides on the target RNA side of the dsRNA substrate. A symmetrical internal loop of the present disclosure may be formed by 800 nucleotides on the engineered polynucleotide side of the dsRNA target and 800 nucleotides on the target RNA side of the dsRNA substrate. A symmetrical internal loop of the present disclosure may be formed by 900 nucleotides on the engineered polynucleotide side of the dsRNA target and 900 nucleotides on the target RNA side of the dsRNA substrate. A symmetrical internal loop of the present disclosure may be formed by 1000 nucleotides on the engineered polynucleotide side of the dsRNA target and 1000 nucleotides on the target RNA side of the dsRNA substrate.

In an aspect, a double stranded RNA (dsRNA) substrate is formed upon hybridization of an engineered polynucleotide of the present disclosure to a target RNA. An internal loop may be a symmetrical internal loop or an asymmetrical internal loop. An asymmetrical internal loop is formed when a different number of nucleotides is present on each side of the internal loop. For example, an asymmetrical internal loop in a dsRNA substrate of the present disclosure may have different numbers of nucleotides on the engineered polynucleotide side and the target RNA side of the dsRNA substrate. An asymmetrical internal loop of the present disclosure may be formed by from 5 to 150 nucleotides on the engineered polynucleotide side of the dsRNA substrate and from 5 to 150 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by from 5 to 1000 nucleotides on the engineered polynucleotide side of the dsRNA substrate and from 5 to 1000 nucleotides on the target RNA side of the dsRNA substrate, wherein the number of nucleotides is the different on the engineered side of the dsRNA target than the number of nucleotides on the target RNA side of the dsRNA substrate. An asymmetrical internal loop of the present disclosure may be formed by 5 nucleotides on the engineered polynucleotide side of the dsRNA substrate and 6 nucleotides on the target RNA side of the dsRNA substrate. An asymmetrical internal loop of the present disclosure may be formed by 5 nucleotides on the target RNA side of the dsRNA substrate and 6 nucleotides on the engineered polynucleotide side of the dsRNA substrate. An asymmetrical internal loop of the present disclosure may be formed by 5 nucleotides on the engineered polynucleotide side of the dsRNA substrate and 7 nucleotides on the target RNA side of the dsRNA substrate. An asymmetrical internal loop of the present disclosure may be formed by 5 nucleotides on the target RNA side of the dsRNA substrate and 7 nucleotides on the engineered polynucleotide side of the dsRNA substrate. An asymmetrical internal loop of the present disclosure may be formed by 5 nucleotides on the engineered polynucleotide side of the dsRNA substrate and 8 nucleotides internal loop the target RNA side of the dsRNA substrate. An asymmetrical internal loop of the present disclosure may be formed by 5 nucleotides on the target RNA side of the dsRNA substrate and 8 nucleotides on the engineered polynucleotide side of the dsRNA substrate. An asymmetrical internal loop of the present disclosure may be formed by 5 nucleotides on the engineered polynucleotide side of the dsRNA substrate and 9 nucleotides internal loop the target RNA side of the dsRNA substrate. An asymmetrical internal loop of the present disclosure may be formed by 5 nucleotides on the target RNA side of the dsRNA substrate and 9 nucleotides on the engineered polynucleotide side of the dsRNA substrate. An asymmetrical internal loop of the present disclosure may be formed by 5 nucleotides on the engineered polynucleotide side of the dsRNA substrate and 10 nucleotides internal loop the target RNA side of the dsRNA substrate. An asymmetrical internal loop of the present disclosure may be formed by 5 nucleotides on the target RNA side of the dsRNA substrate and 10 nucleotides on the engineered polynucleotide side of the dsRNA substrate. An asymmetrical internal loop of the present disclosure may be formed by 6 nucleotides on the engineered polynucleotide side of the dsRNA substrate and 7 nucleotides internal loop the target RNA side of the dsRNA substrate. An asymmetrical internal loop of the present disclosure may be formed by 6 nucleotides on the target RNA side of the dsRNA substrate and 7 nucleotides on the engineered polynucleotide side of the dsRNA substrate. An asymmetrical internal loop of the present disclosure may be formed by 6 nucleotides on the engineered polynucleotide side of the dsRNA substrate and 8 nucleotides internal loop the target RNA side of the dsRNA substrate. An asymmetrical internal loop of the present disclosure may be formed by 6 nucleotides on the target RNA side of the dsRNA substrate and 8 nucleotides on the engineered polynucleotide side of the dsRNA substrate. An asymmetrical internal loop of the present disclosure may be formed by 6 nucleotides on the engineered polynucleotide side of the dsRNA substrate and 9 nucleotides internal loop the target RNA side of the dsRNA substrate. An asymmetrical internal loop of the present disclosure may be formed by 6 nucleotides on the target RNA side of the dsRNA substrate and 9 nucleotides on the engineered polynucleotide side of the dsRNA substrate. An asymmetrical internal loop of the present disclosure may be formed by 6 nucleotides on the engineered polynucleotide side of the dsRNA substrate and 10 nucleotides internal loop the target RNA side of the dsRNA substrate. An asymmetrical internal loop of the present disclosure may be formed by 6 nucleotides on the target RNA side of the dsRNA substrate and 10 nucleotides on the engineered polynucleotide side of the dsRNA substrate. An asymmetrical internal loop of the present disclosure may be formed by 7 nucleotides on the engineered polynucleotide side of the dsRNA substrate and 8 nucleotides internal loop the target RNA side of the dsRNA substrate. An asymmetrical internal loop of the present disclosure may be formed by 7 nucleotides on the target RNA side of the dsRNA substrate and 8 nucleotides on the engineered polynucleotide side of the dsRNA substrate. An asymmetrical internal loop of the present disclosure may be formed by 7 nucleotides on the engineered polynucleotide side of the dsRNA substrate and 9 nucleotides internal loop the target RNA side of the dsRNA substrate. An asymmetrical internal loop of the present disclosure may be formed by 7 nucleotides on the target RNA side of the dsRNA substrate and 9 nucleotides on the engineered polynucleotide side of the dsRNA substrate. An asymmetrical internal loop of the present disclosure may be formed by 7 nucleotides on the engineered polynucleotide side of the dsRNA substrate and 10 nucleotides internal loop the target RNA side of the dsRNA substrate. An asymmetrical internal loop of the present disclosure may be formed by 7 nucleotides on the target RNA side of the dsRNA substrate and 10 nucleotides on the engineered polynucleotide side of the dsRNA substrate. An asymmetrical internal loop of the present disclosure may be formed by 8 nucleotides on the engineered polynucleotide side of the dsRNA substrate and 9 nucleotides internal loop the target RNA side of the dsRNA substrate. An asymmetrical internal loop of the present disclosure may be formed by 8 nucleotides on the target RNA side of the dsRNA substrate and 9 nucleotides on the engineered polynucleotide side of the dsRNA substrate. An asymmetrical internal loop of the present disclosure may be formed by 8 nucleotides on the engineered polynucleotide side of the dsRNA substrate and 10 nucleotides internal loop the target RNA side of the dsRNA substrate. An asymmetrical internal loop of the present disclosure may be formed by 8 nucleotides on the target RNA side of the dsRNA substrate and 10 nucleotides on the engineered polynucleotide side of the dsRNA substrate. An asymmetrical internal loop of the present disclosure may be formed by 9 nucleotides on the engineered polynucleotide side of the dsRNA substrate and 10 nucleotides internal loop the target RNA side of the dsRNA substrate. An asymmetrical internal loop of the present disclosure may be formed by 9 nucleotides on the target RNA side of the dsRNA substrate and 10 nucleotides on the engineered polynucleotide side of the dsRNA substrate. An asymmetrical internal loop of the present disclosure may be formed by 5 nucleotides on the target RNA side of the dsRNA substrate and 50 nucleotides on the engineered polynucleotide side of the dsRNA substrate. An asymmetrical internal loop of the present disclosure may be formed by 5 nucleotides on the target RNA side of the dsRNA substrate and 100 nucleotides on the engineered polynucleotide side of the dsRNA substrate. An asymmetrical internal loop of the present disclosure may be formed by 5 nucleotides on the target RNA side of the dsRNA substrate and 150 nucleotides on the engineered polynucleotide side of the dsRNA substrate. An asymmetrical internal loop of the present disclosure may be formed by 5 nucleotides on the target RNA side of the dsRNA substrate and 200 nucleotides on the engineered polynucleotide side of the dsRNA substrate. An asymmetrical internal loop of the present disclosure may be formed by 5 nucleotides on the target RNA side of the dsRNA substrate and 300 nucleotides on the engineered polynucleotide side of the dsRNA substrate. An asymmetrical internal loop of the present disclosure may be formed by 5 nucleotides on the target RNA side of the dsRNA substrate and 400 nucleotides on the engineered polynucleotide side of the dsRNA substrate. An asymmetrical internal loop of the present disclosure may be formed by 5 nucleotides on the target RNA side of the dsRNA substrate and 500 nucleotides on the engineered polynucleotide side of the dsRNA substrate. An asymmetrical internal loop of the present disclosure may be formed by 5 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 5 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 5 nucleotides on the engineered polynucleotide side of the dsRNA substrate. An asymmetrical internal loop of the present disclosure may be formed by 300 nucleotides on the target RNA side of the dsRNA substrate and 5 nucleotides on the engineered polynucleotide side of the dsRNA substrate. An asymmetrical internal loop of the present disclosure may be formed by 200 nucleotides on the target RNA side of the dsRNA substrate and 5 nucleotides on the engineered polynucleotide side of the dsRNA substrate. An asymmetrical internal loop of the present disclosure may be formed by 150 nucleotides on the target RNA side of the dsRNA substrate and 5 nucleotides on the engineered polynucleotide side of the dsRNA substrate. An asymmetrical internal loop of the present disclosure may be formed by 100 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 5 nucleotides on the engineered polynucleotide side of the dsRNA substrate. An asymmetrical internal loop of the present disclosure may be formed by 50 nucleotides on the target RNA side of the dsRNA substrate and 100 nucleotides on the engineered polynucleotide side of the dsRNA substrate. An asymmetrical internal loop of the present disclosure may be formed by 50 nucleotides on the target RNA side of the dsRNA substrate and 150 nucleotides on the engineered polynucleotide side of the dsRNA substrate. An asymmetrical internal loop of the present disclosure may be formed by 50 nucleotides on the target RNA side of the dsRNA substrate and 200 nucleotides on the engineered polynucleotide side of the dsRNA substrate. An asymmetrical internal loop of the present disclosure may be formed by 50 nucleotides on the target RNA side of the dsRNA substrate and 300 nucleotides on the engineered polynucleotide side of the dsRNA substrate. An asymmetrical internal loop of the present disclosure may be formed by 50 nucleotides on the target RNA side of the dsRNA substrate and 400 nucleotides on the engineered polynucleotide side of the dsRNA substrate. An asymmetrical internal loop of the present disclosure may be formed by 50 nucleotides on the target RNA side of the dsRNA substrate and 500 nucleotides on the engineered polynucleotide side of the dsRNA substrate. An asymmetrical internal loop of the present disclosure may be formed by 50 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 50 nucleotides on the engineered polynucleotide side of the dsRNA substrate. An asymmetrical internal loop of the present disclosure may be formed by 500 nucleotides on the target RNA side of the dsRNA substrate and 50 nucleotides on the engineered polynucleotide side of the dsRNA substrate. An asymmetrical internal loop of the present disclosure may be formed by 400 nucleotides on the target RNA side of the dsRNA substrate and 50 nucleotides on the engineered polynucleotide side of the dsRNA substrate. An asymmetrical internal loop of the present disclosure may be formed by 300 nucleotides on the target RNA side of the dsRNA substrate and 50 nucleotides on the engineered polynucleotide side of the dsRNA substrate. An asymmetrical internal loop of the present disclosure may be formed by 200 nucleotides on the target RNA side of the dsRNA substrate and 50 nucleotides on the engineered polynucleotide side of the dsRNA substrate. An asymmetrical internal loop of the present disclosure may be formed by 150 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 50 nucleotides on the engineered polynucleotide side of the dsRNA substrate. An asymmetrical internal loop of the present disclosure may be formed by 100 nucleotides on the target RNA side of the dsRNA substrate and 150 nucleotides on the engineered polynucleotide side of the dsRNA substrate. An asymmetrical internal loop of the present disclosure may be formed by 100 nucleotides on the target RNA side of the dsRNA substrate and 200 nucleotides on the engineered polynucleotide side of the dsRNA substrate. An asymmetrical internal loop of the present disclosure may be formed by 100 nucleotides on the target RNA side of the dsRNA substrate and 300 nucleotides on the engineered polynucleotide side of the dsRNA substrate. An asymmetrical internal loop of the present disclosure may be formed by 100 nucleotides on the target RNA side of the dsRNA substrate and 400 nucleotides on the engineered polynucleotide side of the dsRNA substrate. An asymmetrical internal loop of the present disclosure may be formed by 100 nucleotides on the target RNA side of the dsRNA substrate and 500 nucleotides on the engineered polynucleotide side of the dsRNA substrate. An asymmetrical internal loop of the present disclosure may be formed by 100 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 100 nucleotides on the engineered polynucleotide side of the dsRNA substrate. An asymmetrical internal loop of the present disclosure may be formed by 500 nucleotides on the target RNA side of the dsRNA substrate and 100 nucleotides on the engineered polynucleotide side of the dsRNA substrate. An asymmetrical internal loop of the present disclosure may be formed by 400 nucleotides on the target RNA side of the dsRNA substrate and 100 nucleotides on the engineered polynucleotide side of the dsRNA substrate. An asymmetrical internal loop of the present disclosure may be formed by 300 nucleotides on the target RNA side of the dsRNA substrate and 100 nucleotides on the engineered polynucleotide side of the dsRNA substrate. An asymmetrical internal loop of the present disclosure may be formed by 200 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 100 nucleotides on the engineered polynucleotide side of the dsRNA substrate. An asymmetrical internal loop of the present disclosure may be formed by 150 nucleotides on the target RNA side of the dsRNA substrate and 200 nucleotides on the engineered polynucleotide side of the dsRNA substrate. An asymmetrical internal loop of the present disclosure may be formed by 150 nucleotides on the target RNA side of the dsRNA substrate and 300 nucleotides on the engineered polynucleotide side of the dsRNA substrate. An asymmetrical internal loop of the present disclosure may be formed by 150 nucleotides on the target RNA side of the dsRNA substrate and 400 nucleotides on the engineered polynucleotide side of the dsRNA substrate. An asymmetrical internal loop of the present disclosure may be formed by 150 nucleotides on the target RNA side of the dsRNA substrate and 500 nucleotides on the engineered polynucleotide side of the dsRNA substrate. An asymmetrical internal loop of the present disclosure may be formed by 150 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 150 nucleotides on the engineered polynucleotide side of the dsRNA substrate. An asymmetrical internal loop of the present disclosure may be formed by 500 nucleotides on the target RNA side of the dsRNA substrate and 5 nucleotides on the engineered polynucleotide side of the dsRNA substrate. An asymmetrical internal loop of the present disclosure may be formed by 400 nucleotides on the target RNA side of the dsRNA substrate and 150 nucleotides on the engineered polynucleotide side of the dsRNA substrate. An asymmetrical internal loop of the present disclosure may be formed by 300 nucleotides on the target RNA side of the dsRNA substrate and 150 nucleotides on the engineered polynucleotide side of the dsRNA substrate. An asymmetrical internal loop of the present disclosure may be formed by 200 nucleotides on the target RNA side of the dsRNA substrate and 300 nucleotides on the engineered polynucleotide side of the dsRNA substrate. An asymmetrical internal loop of the present disclosure may be formed by 200 nucleotides on the target RNA side of the dsRNA substrate and 400 nucleotides on the engineered polynucleotide side of the dsRNA substrate. An asymmetrical internal loop of the present disclosure may be formed by 200 nucleotides on the target RNA side of the dsRNA substrate and 500 nucleotides on the engineered polynucleotide side of the dsRNA substrate. An asymmetrical internal loop of the present disclosure may be formed by 200 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 200 nucleotides on the engineered polynucleotide side of the dsRNA substrate. An asymmetrical internal loop of the present disclosure may be formed by 500 nucleotides on the target RNA side of the dsRNA substrate and 200 nucleotides on the engineered polynucleotide side of the dsRNA substrate. An asymmetrical internal loop of the present disclosure may be formed by 400 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 200 nucleotides on the engineered polynucleotide side of the dsRNA substrate. An asymmetrical internal loop of the present disclosure may be formed by 300 nucleotides on the target RNA side of the dsRNA substrate and 400 nucleotides on the engineered polynucleotide side of the dsRNA substrate. An asymmetrical internal loop of the present disclosure may be formed by 300 nucleotides on the target RNA side of the dsRNA substrate and 500 nucleotides on the engineered polynucleotide side of the dsRNA substrate. An asymmetrical internal loop of the present disclosure may be formed by 300 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 300 nucleotides on the engineered polynucleotide side of the dsRNA substrate. An asymmetrical internal loop of the present disclosure may be formed by 500 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 300 nucleotides on the engineered polynucleotide side of the dsRNA substrate. An asymmetrical internal loop of the present disclosure may be formed by 400 nucleotides on the target RNA side of the dsRNA substrate and 500 nucleotides on the engineered polynucleotide side of the dsRNA substrate. An asymmetrical internal loop of the present disclosure may be formed by 400 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 400 nucleotides on the engineered polynucleotide side of the dsRNA substrate. An asymmetrical internal loop of the present disclosure may be formed by 500 nucleotides on the target RNA side of the dsRNA substrate and 1000 nucleotides on the engineered polynucleotide side of the dsRNA substrate. An asymmetrical internal loop of the present disclosure may be formed by 1000 nucleotides on the target RNA side of the dsRNA substrate and 500 nucleotides on the engineered polynucleotide side of the dsRNA substrate.

Structural features that comprise a bulge or loop can be of any size. In some cases, a bulge or loop comprise at least: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 200, 250, 300, 350, 400, 450, 500, 600, 700, 800, 900, or 1000 bases. In some cases, a bulge or loop comprise at least about 1-10, 5-15, 10-20, 15-25, 20-30, 1-30, 1-40, 1-50, 1-60, 1-70, 1-80, 1-90, 1-100, 1-110, 1-120, 1-130, 1-140, 1-150, 1-200, 1-250, 1-300, 1-350, 1-400, 1-450, 1-500, 1-600, 1-700, 1-800, 1-900, 1-1000, 20-50, 20-60, 20-70, 20-80, 20-90, 20-100, 20-110, 20-120, 20-130, 20-140, 20-150, 1-200, 1-250, 1-300, 1-350, 1-400, 1-450, 1-500, 1-600, 1-700, 1-800, 1-900, 1-1000, 30-40, 30-50, 30-60, 30-70, 30-80, 30-90, 30-100, 30-110, 30-120, 30-130, 30-140, 30-150, 30-200, 30-250, 30-300, 30-350, 30-400, 30-450, 30-500, 30-600, 30-700, 30-800, 30-900, 30-1000, 40-50, 40-60, 40-70, 40-80, 40-90, 40-100, 40-110, 40-120, 40-130, 40-140, 40-150, 40-200, 40-250, 40-300, 40-350, 40-400, 40-450, 40-500, 40-600, 40-700, 40-800, 40-900, 40-1000, 50-60, 50-70, 50-80, 50-90, 50-100, 50-110, 50-120, 50-130, 50-140, 50-150, 50-200, 50-250, 50-300, 50-350, 50-400, 50-450, 50-500, 50-600, 50-700, 50-800, 50-900, 50-1000, 60-70, 60-80, 60-90, 60-100, 60-110, 60-120, 60-130, 60-140, 60-150, 60-200, 60-250, 60-300, 60-350, 60-400, 60-450, 60-500, 60-600, 60-700, 60-800, 60-900, 60-1000, 70-80, 70-90, 70-100, 70-110, 70-120, 70-130, 70-140, 70-150, 70-200, 70-250, 70-300, 70-350, 70-400, 70-450, 70-500, 70-600, 70-700, 70-800, 70-900, 70-1000, 80-90, 80-100, 80-110, 80-120, 80-130, 80-140, 80-150, 80-200, 80-250, 80-300, 80-350, 80-400, 80-450, 80-500, 80-600, 80-700, 80-800, 80-900, 80-1000, 90-100, 90-110, 90-120, 90-130, 90-140, 90-150, 90-200, 90-250, 90-300, 90-350, 90-400, 90-450, 90-500, 90-600, 90-700, 90-800, 90-900, 90-1000, 100-110, 100-120, 100-130, 100-140, 100-150, 100-200, 100-250, 100-300, 100-350, 100-400, 100-450, 100-500, 100-600, 100-700, 100-800, 100-900, 100-1000, 110-120, 110-130, 110-140, 110-150, 110-200, 110-250, 110-300, 110-350, 110-400, 110-450, 110-500, 110-600, 110-700, 110-800, 110-900, 110-1000, 120-130, 120-140, 120-150, 120-200, 120-250, 120-300, 120-350, 120-400, 120-450, 120-500, 120-600, 120-700, 120-800, 120-900, 120-1000, 130-140, 130-150, 130-200, 130-250, 130-300, 130-350, 130-400, 130-450, 130-500, 130-600, 130-700, 130-800, 130-900, 130-1000, 140-150, 140-200, 140-250, 140-300, 140-350, 140-400, 140-450, 140-500, 140-600, 140-700, 140-800, 140-900, 140-1000, 150-200, 150-250, 150-300, 150-350, 150-400, 150-450, 150-500, 150-600, 150-700, 150-800, 150-900, 150-1000, 200-250, 200-300, 200-350, 200-400, 200-450, 200-500, 200-600, 200-700, 200-800, 200-900, 200-1000, 250-300, 250-350, 250-400, 250-450, 250-500, 250-600, 250-700, 250-800, 250-900, 250-1000, 300-350, 300-400, 300-450, 300-500, 300-600, 300-700, 300-800, 300-900, 300-1000, 350-400, 350-450, 350-500, 350-600, 350-700, 350-800, 350-900, 350-1000, 400-450, 400-500, 400-600, 400-700, 400-800, 400-900, 400-1000, 500-600, 500-700, 500-800, 500-900, 500-1000, 600-700, 600-800, 600-900, 600-1000, 700-800, 700-900, 700-1000, 800-900, 800-1000, or 900-1000 bases in total.

In some cases, a structural feature is a structured motif. As disclosed herein, a structured motif comprises two or more structural features in a dsRNA substrate. A structured motif can comprise any combination of structural features, such as in the above claims, to generate an ideal substrate for ADAR editing at a precise location(s). These structural motifs could be artificially engineered to maximized ADAR editing, and/or these structural motifs can be modeled to recapitulate known ADAR substrates.

In some cases, a structural feature comprises an at least partial circularization of a polynucleotide. In some cases, a polynucleotide provided herein can be circularized or in a circular configuration. In some aspects, an at least partially circular polynucleotide lacks a 5′ hydroxyl or a 3′ hydroxyl.

In some embodiments, an engineered polynucleotide having snRNA sequences and snRNA hairpins (e.g., SmOPT sequences and U7 hairpins or variants thereof) or a precursor engineered polynucleotide may not comprise a sequence encoding a sequence configured for RNA interference (RNAi). In some embodiments, an engineered polynucleotide may not comprise a sequence configured for RNAi. In some embodiments, an engineered polynucleotide can comprise a sequence configured for RNAi. In some cases, an engineered polynucleotide may not comprise a sequence encoding a short interfering RNA (siRNA), microRNA (miRNA), short hairpin RNA (shRNA), or Dicer substrate. In some cases, an engineered polynucleotide can comprise a sequence encoding a short interfering RNA (siRNA), microRNA (miRNA), short hairpin RNA (shRNA), or Dicer substrate.

An engineered polynucleotide having snRNA sequences and snRNA hairpins (e.g., SmOPT sequences and U7 hairpins or variants thereof) can be produced from a precursor engineered polynucleotide. In some cases, a precursor engineered polynucleotide can be a precursor engineered linear polynucleotide. In some cases, a precursor engineered polynucleotide can be linear. For example, a precursor engineered polynucleotide can be a linear RNA transcribed from a plasmid. In another example, a precursor engineered polynucleotide can be constructed to be a linear polynucleotide with domains such as a ribozyme domain and a ligation domain that allow for circularization in a cell. The linear polynucleotide with the ligation and ribozyme domains can be transfected into a cell where it can circularize via endogenous cellular enzymes. In some cases, a precursor engineered polynucleotide can be circular. In some cases, a precursor engineered polynucleotide can comprise DNA, RNA or both. In some cases, a precursor engineered polynucleotide can comprise a precursor engineered guide RNA. In some cases, a precursor engineered guide RNA can be used to produce an engineered guide RNA.

An engineered polynucleotide having snRNA sequences and snRNA hairpins (e.g., SmOPT sequences and U7 hairpins or variants thereof), or a precursor engineered polynucleotide as described herein can comprise a spacer domain. In some cases, an engineered polynucleotide or a precursor engineered polynucleotide as described herein may not comprise a spacer domain. In some cases, a spacer domain lower Gibbs free energy (ΔG) of binding of the engineered polynucleotide to a target RNA, relative to a ΔG of binding of a corresponding engineered polynucleotide that lacks the spacer domain, as determined by, for example, Kelvin Probe Force Microscopy (KPFM). In some embodiments, when a targeting sequence at least partially binds to a target RNA, a spacer domain can be separated from the targeting sequence by at least 1 nucleotide, and if the spacer domain binds to the target RNA, the binding of the spacer domain may not produce an edit of the target RNA at the portion of the target RNA that binds to the spacer domain. In some cases, when a spacer domain may be adjacent to a 5′ end or a 3′ end of the targeting sequence, the spacer domain may not be complementary to a target RNA.

In some embodiments, an engineered polynucleotide having snRNA sequences and snRNA hairpins (e.g., SmOPT sequences and U7 hairpins or variants thereof), or a precursor engineered polynucleotide can be more than about: 10, 20, 30, 40, 50, 60, 70, 80, 90, 100, 200, 300, 400, 500, 600, 700, 800, 900, 1000, 2500 or 5000 nucleotides in length. In some embodiments, an engineered polynucleotide (e.g. an engineered guide polynucleotide), or a precursor engineered polynucleotide can be less than about: 20, 30, 40, 50, 60, 70, 80, 90, 100, 200, 300, 400, 500, 600, 700, 800, 900, 1000, 2500 or 5000 nucleotides in length. In some cases, an engineered polynucleotide (e.g. an engineered guide polynucleotide), or a precursor engineered polynucleotide can comprise about: 20 nucleotides to about 5000 nucleotides, 20 nucleotides to about 50 nucleotides, 40 nucleotides to about 80 nucleotides, 70 nucleotides to about 140 nucleotides, 80 nucleotides to about 160 nucleotides, 90 nucleotides to about 200 nucleotides, 100 nucleotides to about 250 nucleotides, 150 nucleotides to about 350 nucleotides, 200 nucleotides to about 500 nucleotides, 450 nucleotides to about 800 nucleotides, 750 nucleotides to about 1250 nucleotides, 1000 nucleotides to about 2000 nucleotides, or about 2000 nucleotides to about 5000 nucleotides.

In some embodiments, a spacer domain can be separated from a targeting sequence by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

In some embodiments an engineered polynucleotide may not comprise a 5′ reducing hydroxyl, a 3′ reducing hydroxyl, or both, capable of being exposed to a solvent. In some instances, an engineered polynucleotide can comprise a secondary structure that can be less susceptible to hydrolytic degradation than an mRNA naturally present in a human cell.

A spacer domain can be configured to facilitate an engineered polynucleotide adopting a conformation that facilitates at least partial binding to a target RNA. In some cases, a spacer domain can change the geometry of a targeting sequence of a polynucleotide so that the targeting sequence of the polynucleotide can be substantially linear. In some embodiments, a spacer domain can facilitate the synthesis of an engineered polynucleotide. In some instances, a spacer domain can facilitate the linkage of the solvent-exposing ends of a precursor engineered polynucleotide. In some embodiments, a spacer domain can bring two ligating ends of a precursor engineered polynucleotide closer than those that lack the spacer domain. In some embodiments, a linkage in an engineered polynucleotide can be covalent or non-covalent. In some cases, a linkage can be formed by a ligation reaction. In some embodiments, a linkage can be formed by a homologous recombination reaction.

An engineered polynucleotide having snRNA sequences and snRNA hairpins (e.g., SmOPT sequences and U7 hairpins or variants thereof) comprising a spacer domain can have an increase in the binding specificity to a target RNA, among a plurality of other RNAs, relative to the binding specificity of a corresponding polynucleotide that lacks the spacer domain. In some embodiments, an increase in the binding specificity to a target RNA can be determined by sequencing of a target RNA and plurality of other RNAs after contacting with an engineered polynucleotide comprising a spacer domain or a corresponding polynucleotide that lacks the spacer domain. In some instances, a spacer domain can be configured to facilitate a lower entropy (ΔS) of binding of an engineered polynucleotide to a target RNA. In some embodiments, a spacer domain can be configured to at least maintain an editing efficiency of an engineered polynucleotide to a target RNA, relative to the editing efficiency of a corresponding polynucleotide that lacks the spacer domain. In some cases, an editing efficiency can be determined by sequencing of a target RNA after contacting with an engineered polynucleotide with a spacer domain or a corresponding polynucleotide that lacks the spacer domain. In some embodiments, an at least maintain can comprise an increase. In some instances, an editing efficiency can be determined by mass spectroscopy of a target RNA after contacting with an engineered polynucleotide with a spacer domain or a corresponding polynucleotide that lacks the spacer domain.

An engineered guide RNA, an engineered polynucleotide, or a precursor engineered linear polynucleotide encoding an engineered polynucleotide having snRNA sequences and snRNA hairpins (e.g., SmOPT sequences and U7 hairpins or variants thereof), can facilitate an edit of a target RNA, for example, via an RNA editing entity. In some cases, an engineered polynucleotide may not facilitate an edit of a target RNA. In some instances, an engineered polynucleotide can have an increased editing efficiency to a target RNA by at least about 90%, relative to an otherwise comparable polynucleotide that can comprise a 5′ reducing hydroxyl, a 3′ reducing hydroxyl, or both. In some embodiments, an editing efficiency can be determined by (i) transfecting a target RNA into a primary cell line, (ii) transfecting an engineered polynucleotide and an otherwise comparable polynucleotide that can comprise a 5′ reducing hydroxyl, a 3′ reducing hydroxyl or both, into a primary cell line, and (iii) sequencing the target RNA. In some embodiments, an editing efficiency can be determined by (i) transfecting a target RNA into a primary cell line, (ii) transfecting an engineered polynucleotide and an otherwise comparable polynucleotide that can comprise the 5′ reducing hydroxyl, the 3′ reducing hydroxyl or both, into a primary cell line, and (iii) mass spectroscopy of the target RNA. In some embodiments, an edit of a base of a nucleotide of a target RNA by an RNA editing entity can be determined in an in vitro assay comprising: (i) directly or indirectly introducing (e.g. transfecting) the target RNA into a primary cell line, (ii) directly or indirectly introducing (e.g. transfecting) the engineered polynucleotide into a primary cell line, and (iii) sequencing the target RNA. In some cases, transfecting the target RNA into a primary cell line can comprise transfecting a plasmid encoding for the target RNA into a primary cell line. In some instances, transfecting an engineered polynucleotide into a primary cell line can comprise transfecting a precursor engineered polynucleotide, or a polynucleotide (e.g. plasmid) that encodes for a precursor engineered polynucleotide into a primary cell line. In some cases, sequencing can comprise Sanger sequencing of a target RNA after the target RNA has been converted to cDNA by reverse transcriptase. In some instances, a primary cell line can comprise a neuron, a photoreceptor cell, a retinal pigment epithelium cell, a glia cell, a myoblast cell, a myotube cell, a hepatocyte, a lung epithelial cell, or a fibroblast cell.

In some cases, a spacer domain can have a sequence length of from about: 1 nucleotide to about 1,000 nucleotides, 2 nucleotides to about 20 nucleotides, 10 nucleotides to about 100 nucleotides, 50 nucleotides to about 500 nucleotides or about 400 nucleotides to about 1000 nucleotides in length. In some embodiments, about 80% of the nucleotides of a spacer domain can be non-complementary to the target RNA. In some cases, a spacer domain can have a sequence length of about 5 nucleotides. In some cases, a spacer domain can have a sequence length of about 10 nucleotides. In some cases, a spacer domain can have a sequence length of about 15 nucleotides. In some cases, a spacer domain can have a sequence length of about 20 nucleotides. In some embodiments, a spacer domain can comprise a polynucleotide sequence of ATATA (SEQ ID NO: 50), ATAAT (SEQ ID NO: 51), or any combination thereof. In some cases, a spacer domain can comprise a sequence of AUAAU (SEQ ID NO: 52), AUAUA (SEQ ID NO: 53), or UAAUA (SEQ ID NO: 54). In some embodiments, a spacer domain can be at least a single nucleotide, such as A, T, G, C or U.

A spacer domain can be located proximal to a targeting sequence, proximal to a ligation domain, proximal to a ribozyme domain, proximal to a RNA editing recruiting domain, or proximal to another spacer domain, where proximal can mean separated by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 nucleotides.

An engineered polynucleotide, or a precursor engineered polynucleotide can comprise a single spacer domain. In some embodiments an engineered polynucleotide, or a precursor engineered polynucleotide can comprise multiple spacer domains, for example 2, 3, 4, 5, 6, 7, or 8 spacer domains. In some instances, an engineered polynucleotide, or a precursor engineered polynucleotide can comprise one targeting sequence. In some cases, an engineered polynucleotide, or a precursor engineered polynucleotide can comprise more than one targeting sequence. In some embodiments, an engineered polynucleotide, or a precursor engineered polynucleotide can comprise two targeting sequences. In some instances, an engineered polynucleotide, or a precursor engineered polynucleotide can comprise two targeting sequences that target a same target RNA. In some instances, an engineered polynucleotide, or a precursor engineered polynucleotide can comprise two targeting sequences that target different target RNAs. In some instances, an engineered polynucleotide, or a precursor engineered polynucleotide can comprise two targeting sequences that comprise a same polynucleotide sequence identity. In some instances, an engineered polynucleotide, or a precursor engineered polynucleotide can comprise two targeting sequences that comprise different polynucleotide sequence identities.

An in vitro half-life of an engineered guide RNA, an engineered polynucleotide, or a precursor engineered polynucleotide with a spacer domain can be at least about: 1×, 1.5×, 2×, 2.5×, 3×, 3.5×, 4×, 5×, 10×, 20×longer or more as compared to a substantially comparable RNA or comparable polynucleotide that lacks the spacer domain. An in vivo half-life of an engineered guide RNA, an engineered polynucleotide, or a precursor engineered polynucleotide with a spacer domain can be at least about: 1×, 1.5×, 2×, 2.5×, 3×, 3.5×, 4×, 5×, 10×, 20× longer or more as compared to a substantially comparable RNA or comparable polynucleotide that lacks the spacer domain. A dosage of a composition comprising an engineered guide RNA, an engineered polynucleotide, or a precursor engineered polynucleotide with a spacer domain administered to a subject in need thereof can be at least about: lx, 1.5×, 2×, 2.5×, 3×, 3.5×, 4×, 5×, 10×, or 20× less as compared to a composition comprising a substantially comparable RNA or comparable polynucleotide that lacks the spacer domain administered to a subject in need thereof. A composition comprising an engineered guide RNA, an engineered polynucleotide, or a precursor engineered polynucleotide with a spacer domain administered to a subject in need thereof can be given as a single time treatment as compared to a composition comprising a substantially comparable RNA or comparable polynucleotide that lacks the spacer domain given as a two-time treatment or more.

In some embodiments, the engineered polynucleotide can comprise at least one chemical modification. In some embodiments, the engineered polynucleotide can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, or more chemical modifications. In some embodiments, the engineered polynucleotide comprises no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 chemical modifications. In some embodiments, the engineered polynucleotide can comprise at least 1, 2, 3, 4, 5, 6, 7, 8, 9, 10, 50, 100, or more chemically modified nucleotides at the 5′ end of the engineered polynucleotide. In some embodiments, the engineered polynucleotide can comprise at least 1, 2, 3, 4, 5, 6, 7, 8, 9, 10, 50, 100, or more chemically modified nucleotides at the 3′ end of the engineered polynucleotide. In some embodiments, the engineered polynucleotide can comprise at least 1, 2, 3, 4, 5, 6, 7, 8, 9, 10, 50, 100, or more chemically modified nucleotides at both the 5′ and the 3′ end of the engineered polynucleotide. In some embodiments, the engineered polynucleotide can comprise at least one chemical modification in the targeting sequence of the engineered polynucleotide. In some embodiments, the engineered polynucleotide can comprise at least one chemical modification in the nucleotide bases adjacent the targeting sequence. In some embodiments, the at least one chemical modification can be introduced within an intramolecular secondary structure.

In some embodiments, the chemical modifications of the engineered polynucleotide can comprise at least one chemical modification to one or both of non-linking phosphate oxygen atoms in a phosphodiester backbone linkage of the engineered polynucleotide. In some embodiments, the at least one chemical modification of the engineered polynucleotide can comprise a chemical modification to one or more of linking phosphate oxygen atoms in a phosphodiester backbone linkage of the engineered polynucleotide. In some embodiments, the chemical modifications of the engineered polynucleotide can comprise at least one chemical modification to a sugar of a nucleotide of the engineered polynucleotide. In some embodiments, the chemical modifications of the engineered polynucleotide can comprise at least one chemical modification to the sugar of the nucleotide of the engineered polynucleotide comprising at least one locked nucleic acid (LNA). In some embodiments, the chemical modifications of the engineered polynucleotide can comprise at least one chemical modification to the sugar of the nucleotide of the engineered polynucleotide comprising at least one unlocked nucleic acid (UNA). In some embodiments, the chemical modifications of the engineered polynucleotide can comprise at least one chemical modification to the sugar comprising a modification of a constituent of the sugar, where the sugar is a ribose sugar. In some embodiments, the chemical modifications of the engineered polynucleotide can comprise at least one chemical modification to the constituent of the ribose sugar of the nucleotide of the engineered polynucleotide comprising a 2′-O-Methyl group. In some embodiments, the chemical modifications of the engineered polynucleotide can comprise at least one chemical modification comprising replacement of a phosphate moiety of the engineered polynucleotide with a dephospho linker. In some embodiments, the chemical modifications of the engineered polynucleotide can comprise at least one chemical modification of a phosphate backbone of the engineered polynucleotide. In some embodiments, the engineered polynucleotide can comprise a phosphorothioate group. In some embodiments, the chemical modifications of the engineered polynucleotide can comprise at least one chemical modification comprising a modification to a base of a nucleotide of the engineered polynucleotide. In some embodiments, the chemical modifications of the engineered polynucleotide can comprise at least one chemical modification comprising an unnatural base of a nucleotide. In some embodiments, the chemical modifications of the engineered polynucleotide can comprise at least one chemical modification comprising a morpholino group, a cyclobutyl group, pyrrolidine group, or peptide nucleic acid (PNA) nucleoside surrogate. In some embodiments, the chemical modifications of the engineered polynucleotide can comprise at least one chemical modification comprising at least one stereopure nucleic acid. In some embodiments, the at least one chemical modification can be positioned proximal to a 5′ end of the engineered polynucleotide. In some embodiments, the at least one chemical modification can be positioned proximal to a 3′ end of the engineered polynucleotide. In some embodiments, the at least one chemical modification can be positioned proximal to both 5′ and 3′ ends of the engineered polynucleotide.

In some embodiments, an engineered polynucleotide can comprise a backbone comprising a plurality of sugar and phosphate moieties covalently linked together. In some cases, a backbone of an engineered polynucleotide can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered polynucleotide can lack a 5′ reducing hydroxyl, a 3′ reducing hydroxyl, or both, capable of being exposed to a solvent. In some embodiments, a backbone of an engineered polynucleotide can lack a 5′ reducing hydroxyl, a 3′ reducing hydroxyl, or both, capable of being exposed to nucleases. In some embodiments, a backbone of an engineered polynucleotide can lack a 5′ reducing hydroxyl, a 3′ reducing hydroxyl, or both, capable of being exposed to hydrolytic enzymes. In some instances, a backbone of an engineered polynucleotide can be represented as a polynucleotide sequence in a circular 2-dimensional format with one nucleotide after the other. In some instances, a backbone of an engineered polynucleotide can be represented as a polynucleotide sequence in a looped 2-dimensional format with one nucleotide after the other. In some cases, a 5′ hydroxyl, a 3′ hydroxyl, or both, are joined through a phosphorus-oxygen bond. In some cases, a 5′ hydroxyl, a 3′ hydroxyl, or both, are modified into a phosphoester with a phosphorus-containing moiety.

In some embodiments, the engineered polynucleotide described herein can comprise at least one chemical modification. A chemical modification can be a substitution, insertion, deletion, chemical modification, physical modification, stabilization, purification, or any combination thereof. In some cases, a modification is a chemical modification. Suitable chemical modifications comprise any one of: 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′deoxyribonucleoside analog purine, 2′deoxyribonucleoside analog pyrimidine, ribonucleoside analog, 2′-O-methyl ribonucleoside analog, sugar modified analogs, wobble/universal bases, fluorescent dye label, 2′fluoro RNA, 2′O-methyl RNA, methylphosphonate, phosphodiester DNA, phosphodiester RNA, phosphorothioate DNA, phosphorothioate RNA, UNA, pseudouridine-5′-triphosphate, 5-methylcytidine-5′-triphosphate, 2-O-methyl 3phosphorothioate or any combinations thereof.

A chemical modification can be made at any location of the engineered polynucleotide. In some cases, a modification is located in a 5′ or 3′ end. In some cases, a polynucleotide comprises a modification at a base selected from: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, or 150. More than one modification can be made to the engineered polynucleotide. In some cases, a modification can be permanent. In other cases, a modification can be transient. In some cases, multiple modifications are made to the engineered polynucleotide. The engineered polynucleotide modification can alter physio-chemical properties of a nucleotide, such as their conformation, polarity, hydrophobicity, chemical reactivity, base-pairing interactions, or any combination thereof.

A chemical modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a polynucleic acid. A modification can also enhance biological activity. In some cases, a phosphorothioate enhanced RNA polynucleic acid can inhibit RNase A, RNase T1, calf serum nucleases, or any combinations thereof. These properties can allow the use of PS-RNA polynucleic acids to be used in applications where exposure to nucleases is of high probability in vivo or in vitro. For example, phosphorothioate (PS) bonds can be introduced between the last 3-5 nucleotides at the 5′- or 3′-end of a polynucleic acid which can inhibit exonuclease degradation. In some cases, phosphorothioate bonds can be added throughout an entire polynucleic acid to reduce attack by endonucleases.

The engineered polynucleotide described herein can have any frequency of bases. For example, a polynucleotide can have a percent adenine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent cytosine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent thymine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent guanine of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%. A polynucleotide can have a percent uracil of 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 1-5%, 3-8%, 5-12%, 10-15%, 8-20%, 15-25%, 20-30%, 25-35%, or up to about 30-40%.

In some cases, the engineered polynucleotide can undergo quality control after a modification. In some cases, quality control may include PAGE, HPLC, MS, or any combination thereof. In some cases, a mass of a polynucleotide can be determined. A mass can be determined by LC-MS assay. A mass can be 30,000 amu, 50,000 amu, 70,000 amu, 90,000 amu, 100,000 amu, 120,000 amu, 150,000 amu, 175,000 amu, 200,000 amu, 250,000 amu, 300,000 amu, 350,000 amu, 400,000 amu, to about 500,000 amu. A mass can be of a sodium salt of a polynucleotide.

In some cases, an endotoxin level of a polynucleotide can be determined. A clinically/therapeutically acceptable level of an endotoxin can be less than 3 EU/mL. A clinically/therapeutically acceptable level of an endotoxin can be less than 10 EU/mL. A clinically/therapeutically acceptable level of an endotoxin can be less than 8 EU/mL. A clinically/therapeutically acceptable level of an endotoxin can be less than 5 EU/mL. A clinically/therapeutically acceptable level of an endotoxin can be less than 4 EU/mL. A clinically/therapeutically acceptable level of an endotoxin can be less than 3 EU/mL. A clinically/therapeutically acceptable level of an endotoxin can be less than 2 EU/mL. A clinically/therapeutically acceptable level of an endotoxin can be less than 1 EU/mL. A clinically/therapeutically acceptable level of an endotoxin can be less than 0.5 EU/mL.

In some embodiments, engineered polynucleotides described herein can comprise at least one chemical modification. In some embodiments, the engineered polynucleotide comprises at least one, two, three, four, five, six, seven, eight, nine, ten, 11, 12, 13, 14, 15, 16, 17, 18, 19, 20, 30, 50, 100, or more chemical modifications.

In some embodiments, chemical modification can occur at 3′OH, group, 5′OH group, at the backbone, at the sugar component, or at the nucleotide base. Chemical modification can include non-naturally occurring linker molecules of interstrand or intrastrand cross links. In one aspect, the chemically modified nucleic acid comprises modification of one or more of the 3′OH or 5′OH group, the backbone, the sugar component, or the nucleotide base, or addition of non-naturally occurring linker molecules. In some embodiments, chemically modified backbone comprises a backbone other than a phosphodiester backbone. In some embodiments, a modified sugar comprises a sugar other than deoxyribose (in modified DNA) or other than ribose (modified RNA). In some embodiments, a modified base comprises a base other than adenine, guanine, cytosine, thymine or uracil. In some embodiments, the engineered polynucleotide comprises at least one chemically modified base. In some instances, the engineered polynucleotide comprises 2, 3, 4, 5, 6, 7, 8, 9, 10, 15, 20, or more modified bases. In some cases, chemical modifications to the base moiety include natural and synthetic modifications of adenine, guanine, cytosine, thymine, or uracil, and purine or pyrimidine bases.

In some embodiments, chemical modification of the engineered polynucleotide can comprise a modification of any one of or any combination of: modification of one or both of the non-linking phosphate oxygens in the phosphodiester backbone linkage; modification of one or more of the linking phosphate oxygens in the phosphodiester backbone linkage; modification of a constituent of the ribose sugar; Replacement of the phosphate moiety with “dephospho” linkers; modification or replacement of a naturally occurring nucleobase; modification of the ribose-phosphate backbone; modification of 5′ end of polynucleotide; modification of 3′ end of polynucleotide; modification of the deoxyribose phosphate backbone; substitution of the phosphate group; modification of the ribophosphate backbone; modifications to the sugar of a nucleotide; modifications to the base of a nucleotide; or stereopure of nucleotide. Exemplary chemical modification to the engineered polynucleotide can be seen in TABLE 1.

TABLE 1
Exemplary Chemical Modification

| Modification of engineered | |
|---|---|
| polynucleotide | Examples |
| Modification of one or both of | sulfur (S), selenium (Se), BR₃ (wherein R can be, e.g., |
| the non-linking phosphate | hydrogen, alkyl, or aryl), C (e.g., an alkyl group, an aryl group, |
| oxygens in the phosphodiester | and the like), H, NR₂, wherein R can be, e.g., hydrogen, alkyl, |
| backbone linkage | or aryl, or wherein R can be, e.g., alkyl or aryl |
| Modification of one or more of | sulfur (S), selenium (Se), BR₃ (wherein R can be, e.g., |
| the linking phosphate oxygens | hydrogen, alkyl, or aryl), C (e.g., an alkyl group, an aryl group, |
| in the phosphodi ester backbone | and the like), H, NR₂, wherein R can be, e.g., hydrogen, alkyl, |
| linkage | or aryl, or wherein R can be, e.g., alkyl or aryl |
| Replacement of the phosphate | methyl phosphonate, hydroxylamino, siloxane, carbonate, |
| moiety with “dephospho” | carboxymethyl, carbamate, amide, thioether, ethylene oxide |
| linkers | linker, sulfonate, sulfonamide, thioformacetal, formacetal, |
| | oxime, methyleneimino, methylenemethylimino, |
| | methylenehydrazo, methylenedimethylhydrazo, or |
| | methyleneoxymethylimino |
| Modification or replacement of | Nucleic acid analog (examples of nucleotide analogs can be |
| a naturally occurring | found in PCT/US2015/025175, PCT/US2014/050423, |
| nucleobase | PCT/US2016/067353, PCT/US2018/041503, |
| | PCT/US18/041509, PCT/US2004/011786, or |
| | PCT/US2004/011833, all of which are expressly incorporated |
| | by reference in their entireties |
| Modification of the ribose- | phosphorothioate, phosphonothioacetate, phosphoroselenates, |
| phosphate backbone | boranophosphates, borano phosphate esters, hydrogen |
| | phosphonates, phosphonocarboxylate, phosphoroamidates, |
| | alkyl or aryl phosphonates, phosphonoacetate, or |
| | phosphotriesters |
| Modification of 5′ end of | 5′ cap or modification of 5′ cap —OH |
| polynucleotide | |
| Modification of 3′ end of | 3′ tail or modification of 3′ end —OH |
| polynucleotide | |
| Modification of the | phosphorothioate, phosphonothioacetate, phosphoroselenates, |
| deoxyribose phosphate | borano phosphates, borano phosphate esters, hydrogen |
| backbone | phosphonates, phosphoroamidates, alkyl or aryl phosphonates, |
| | or phosphotriesters |
| Substitution of the phosphate | methyl phosphonate, hydroxylamino, siloxane, carbonate, |
| group | carboxymethyl, carbamate, amide, thioether, ethylene oxide |
| | linker, sulfonate, sulfonamide, thioformacetal, formacetal, |
| | oxime, methyleneimino, methylenemethylimino, |
| | methylenehydrazo, methylenedimethylhydrazo, or |
| | methyleneoxymethylimino. |
| Modification of the | morpholino, cyclobutyl, pyrrolidine, or peptide nucleic acid |
| ribophosphate backbone | (PNA) nucleoside surrogates |
| Modifications to the sugar of a | Locked nucleic acid (LNA), unlocked nucleic acid (UNA), or |
| nucleotide | bridged nucleic acid (BNA) |
| Modification of a constituent of | 2′-O-methyl, 2′-O-methoxy-ethyl (2′-MOE), 2′-fluoro, 2′- |
| the ribose sugar | aminoethyl, 2′-deoxy-2′-fuloarabinou-cleic acid, 2′-deoxy, 2′- |
| | O-methyl, 3′-phosphorothioate, 3′-phosphonoacetate (PACE), |
| | or 3′-phosphonothioacetate (thioPACE) |
| Modifications to the base of a | Modification of A, T, C, G, or U |
| nucleotide | |
| Stereopure of nucleotide | S conformation of phosphorothioate or R conformation of |
| | phosphorothioate |

In some embodiments, the chemical modification comprises modification of one or both of the non-linking phosphate oxygens in the phosphodiester backbone linkage or modification of one or more of the linking phosphate oxygens in the phosphodiester backbone linkage. As used herein, “alkyl” is meant to refer to a saturated hydrocarbon group which is straight-chained or branched. Example alkyl groups include methyl (Me), ethyl (Et), propyl (e.g., n-propyl or isopropyl), butyl (e.g., n-butyl, isobutyl, or t-butyl), or pentyl (e.g., n-pentyl, isopentyl, or neopentyl). An alkyl group can contain from 1 to about 20, from 2 to about 20, from 1 to about 12, from 1 to about 8, from 1 to about 6, from 1 to about 4, or from 1 to about 3 carbon atoms. As used herein, “aryl” refers to monocyclic or polycyclic (e.g., having 2, 3, or 4 fused rings) aromatic hydrocarbons such as, for example, phenyl, naphthyl, anthracenyl, phenanthrenyl, indanyl, or indenyl. In some embodiments, aryl groups have from 6 to about 20 carbon atoms. As used herein, “alkenyl” refers to an aliphatic group containing at least one double bond. As used herein, “alkynyl” refers to a straight or branched hydrocarbon chain containing 2-12 carbon atoms and characterized in having one or more triple bonds. Examples of alkynyl groups can include ethynyl, propargyl, or 3-hexynyl. “Arylalkyl” or “aralkyl” refers to an alkyl moiety in which an alkyl hydrogen atom is replaced by an aryl group. Aralkyl includes groups in which more than one hydrogen atom has been replaced by an aryl group. Examples of “arylalkyl” or “aralkyl” include benzyl, 2-phenylethyl, 3-phenylpropyl, 9-fluorenyl, benzhydryl, and trityl groups. “Cycloalkyl” refers to a cyclic, bicyclic, tricyclic, or polycyclic non-aromatic hydrocarbon groups having 3 to 12 carbons. Examples of cycloalkyl moieties include, but are not limited to, cyclopropyl, cyclopentyl, and cyclohexyl. “Heterocyclyl” refers to a monovalent radical of a heterocyclic ring system. Representative heterocyclyls include, without limitation, tetrahydrofuranyl, tetrahydrothienyl, pyrrolidinyl, pyrrolidonyl, piperidinyl, pyrrolinyl, piperazinyl, dioxanyl, dioxolanyl, diazepinyl, oxazepinyl, thiazepinyl, and morpholinyl. “Heteroaryl” refers to a monovalent radical of a heteroaromatic ring system. Examples of heteroaryl moieties can include imidazolyl, oxazolyl, thiazolyl, triazolyl, pyrrolyl, furanyl, indolyl, thiophenyl pyrazolyl, pyridinyl, pyrazinyl, pyridazinyl, pyrimidinyl, indolizinyl, purinyl, naphthyridinyl, quinolyl, and pteridinyl.

In some embodiments, the phosphate group of a chemically modified nucleotide can be modified by replacing one or more of the oxygens with a different substituent. In some embodiments, the chemically modified nucleotide can include replacement of an unmodified phosphate moiety with a modified phosphate as described herein. In some embodiments, the modification of the phosphate backbone can include alterations that result in either an uncharged linker or a charged linker with unsymmetrical charge distribution. Examples of modified phosphate groups can include phosphorothioate, phosphonothioacetate, phosphoroselenates, boranophosphates, boranophosphate esters, hydrogen phosphonates, phosphoroamidates, alkyl or aryl phosphonates and phosphotriesters. In some embodiments, one of the non-bridging phosphate oxygen atoms in the phosphate backbone moiety can be replaced by any of the following groups: sulfur (S), selenium (Se), BR₃ (wherein R can be, e.g., hydrogen, alkyl, or aryl), C (e.g., an alkyl group, an aryl group, and the like), H, NR₂ (wherein R can be, e.g., hydrogen, alkyl, or aryl), or (wherein R can be, e.g., alkyl or aryl). The phosphorous atom in an unmodified phosphate group can be achiral. However, replacement of one of the non-bridging oxygens with one of the above atoms or groups of atoms can render the phosphorous atom chiral. A phosphorous atom in a phosphate group modified in this way is a stereogenic center. The stereogenic phosphorous atom can possess either the “R” configuration (herein Rp) or the “S” configuration (herein Sp). In some cases, the engineered polynucleotide can comprise stereopure nucleotides comprising S conformation of phosphorothioate or R conformation of phosphorothioate. In some embodiments, the chiral phosphate product is present in a diastereomeric excess of 50%, 60%, 70%, 80%, 90%, or more. In some embodiments, the chiral phosphate product is present in a diastereomeric excess of 95%. In some embodiments, the chiral phosphate product is present in a diastereomeric excess of 96%. In some embodiments, the chiral phosphate product is present in a diastereomeric excess of 97%. In some embodiments, the chiral phosphate product is present in a diastereomeric excess of 98%. In some embodiments, the chiral phosphate product is present in a diastereomeric excess of 99%. In some embodiments, both non-bridging oxygens of phosphorodithioates can be replaced by sulfur. The phosphorus center in the phosphorodithioates can be achiral which precludes the formation of oligoribonucleotide diastereomers. In some embodiments, modifications to one or both non-bridging oxygens can also include the replacement of the non-bridging oxygens with a group independently selected from S, Se, B, C, H, N, and OR (R can be, e.g., alkyl or aryl). In some embodiments, the phosphate linker can also be modified by replacement of a bridging oxygen, (e.g., the oxygen that links the phosphate to the nucleoside), with nitrogen (bridged phosphoroamidates), sulfur (bridged phosphorothioates) and carbon (bridged methylenephosphonates). The replacement can occur at either or both of the linking oxygens.

The engineered polynucleotide can include a pre-mRNA targeting sequence. The pre-mRNA targeting sequence may not be completely complementary to the target pre-mRNA. The targeting sequence can be at least partially complementary to a pre-mRNA species. The targeting sequence can be at least partially complementary to a splice signal proximal to an exon within the pre-mRNA. The targeting sequence can comprise at least one, at least two, at least three, at least four, at least five, at least ten, at least twenty nucleotides that are not complementary to the pre-mRNA.

The targeting sequence can be at least partially complementary to a branch point upstream of an exon within the pre-mRNA. The targeting sequence can be at least partially complementary to a donor splice site downstream of an exon within the pre-mRNA.

In some embodiments, the secondary structure comprises a hairpin. In some embodiments, the hairpin comprises at least about 80%, at least about 85%, at least about 90%, at least about 92%, at least about 95%, at least about 97%, or at least about 99% sequence identity to the stem-loop hairpin of mouse or human U7 snRNA. In some embodiments, the hairpin is the stem-loop hairpin of mouse or human U7 snRNA.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 1. In some embodiments, the engineered polynucleotide is SEQ ID NO: 1.

In some embodiments, the engineered polynucleotide comprises at least 75%, at least 80%, at least 85%, at least 90%, at least 95%, at least 96%, at least 97%, at least 98%, at least 99%, or 100% sequence identity to SEQ ID NO: 2. In some embodiments, the engineered polynucleotide is SEQ ID NO: 2.

In some embodiments, the engineered polynucleotide comprises at least 75%, at least 80%, at least 85%, at least 90%, at least 95%, at least 96%, at least 97%, at least 98%, at least 99%, or 100% sequence identity to SEQ ID NO: 3. In some embodiments, the engineered polynucleotide is SEQ ID NO: 3.

In some embodiments, engineered polynucleotide comprises at least 75%, at least 80%, at least 85%, at least 90%, at least 95%, at least 96%, at least 97%, at least 98%, at least 99%, or 100% sequence identity to SEQ ID NO: 5. In some embodiments, the engineered polynucleotide is SEQ ID NO: 5.

In some embodiments, the engineered polynucleotide comprises at least 75%, at least 80%, at least 85%, at least 90%, at least 95%, at least 96%, at least 97%, at least 98%, at least 99%, or 100% sequence identity to SEQ ID NO: 6. In some embodiments, the engineered polynucleotide is SEQ ID NO: 6.

In some embodiments, the engineered polynucleotide comprises at least 75%, at least 80%, at least 85%, at least 90%, at least 95%, at least 96%, at least 97%, at least 98%, at least 99%, or 100% sequence identity to SEQ ID NO: 7. In some embodiments, the engineered polynucleotide is SEQ ID NO: 7.

In some embodiments, the engineered polynucleotide comprises at least 75%, at least 80%, at least 85%, at least 90%, at least 95%, at least 96%, at least 97%, at least 98%, at least 99%, or 100% sequence identity to SEQ ID NO: 8. In some embodiments, the engineered polynucleotide is SEQ ID NO: 8.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 9. In some embodiments, the engineered polynucleotide is SEQ ID NO: 9.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 10. In some embodiments, the engineered polynucleotide is SEQ ID NO: 10.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 11. In some embodiments, the engineered polynucleotide is SEQ ID NO: 11.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 12. In some embodiments, the engineered polynucleotide is SEQ ID NO: 12.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 13. In some embodiments, the engineered polynucleotide is SEQ ID NO: 13.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 14. In some embodiments, the engineered polynucleotide is SEQ ID NO: 14.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 15. In some embodiments, the engineered polynucleotide is SEQ ID NO: 15.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 16. In some embodiments, the engineered polynucleotide is SEQ ID NO: 16.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 17. In some embodiments, the engineered polynucleotide is SEQ ID NO: 17.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 18. In some embodiments, the engineered polynucleotide is SEQ ID NO: 18.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 19. In some embodiments, the engineered polynucleotide is SEQ ID NO: 19.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 20. In some embodiments, the engineered polynucleotide is SEQ ID NO: 20.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 21. In some embodiments, the engineered polynucleotide is SEQ ID NO: 21.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 22. In some embodiments, the engineered polynucleotide is SEQ ID NO: 22.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 23. In some embodiments, the engineered polynucleotide is SEQ ID NO: 23.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 24. In some embodiments, the engineered polynucleotide is SEQ ID NO: 24.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 25. In some embodiments, the engineered polynucleotide is SEQ ID NO: 25.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 26. In some embodiments, the engineered polynucleotide is SEQ ID NO: 26.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 27. In some embodiments, the engineered polynucleotide is SEQ ID NO: 27.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 29. In some embodiments, the engineered polynucleotide is SEQ ID NO: 29.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 30. In some embodiments, the engineered polynucleotide is SEQ ID NO: 30.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 31. In some embodiments, the engineered polynucleotide is SEQ ID NO: 31.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 32. In some embodiments, the engineered polynucleotide is SEQ ID NO: 32.

In some embodiments, the engineered polynucleotide sequence comprises at least 75%, at least 80%, at least 85%, at least 90%, at least 95%, at least 96%, at least 97%, at least 98%, at least 99%, or 100% sequence identity to SEQ ID NO: 33. In some embodiments, the engineered polynucleotide is SEQ ID NO: 33.

RNA editing of the pre-mRNA is facilitated by targeting of the engineered polynucleotide comprising a targeting sequence. The targeting sequence can be capable of binding to an at least partially complementary target region of the pre-mRNA.

The target region can comprise a splice signal. The target region can be proximal to an exon, intron, or promoter.

In some embodiments, the engineered polynucleotide targets a region of the gene that is implicated in disease or condition. The region is an exon. In some embodiments, the region is an intron. An engineered polynucleotide having snRNA sequences and snRNA hairpins (e.g., SmOPT sequences and U7 hairpins or variants thereof) as described herein can be administered to a subject to treat a disease or condition. Such a disease or condition can comprise a neurodegenerative disease, a muscular disorder, a metabolic disorder, an ocular disorder (e.g. an ocular disease), a cancer, a liver disease (Alpha-1 antitrypsin (AAT) deficiency), or any combination thereof. The disease or condition can comprise cystic fibrosis, albinism, alpha-1-antitrypsin deficiency, Alzheimer disease, Amyotrophic lateral sclerosis, Asthma, β-thalassemia, Cadasil syndrome, Charcot-Marie-Tooth disease, Chronic Obstructive Pulmonary Disease (COPD), dementia, Distal Spinal Muscular Atrophy (DSMA), Duchenne/Becker muscular dystrophy, Dystrophic Epidermolysis bullosa, Epidermylosis bullosa, Fabry disease, Factor V Leiden associated disorders, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C, NY-esol related cancer, Parkinson's disease, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, Usher syndrome, X-linked immunodeficiency, various forms of cancer (e.g., BRCA1 and 2 linked breast cancer and ovarian cancer). In some cases, a treatment of a disease or condition such as a neurodegenerative disease (e.g. Alzheimer's, Parkinson's) can comprise producing an edit, a knockdown or both of amyloid precursor protein (APP), tau, alpha-synuclein, or any combination thereof. In some cases, APP, tau, and alpha-synuclein can comprise a pathogenic variant. In some instances, APP can comprise a pathogenic variant such as A673V mutation or A673T mutation. In some cases, a treatment of a disease or condition such as a neurodegenerative disease (Parkinson's) can comprise producing an edit, a knockdown or both of a pathogenic variant of LRRK2. In some cases, a pathogenic variant of LRRK can comprise a G2019S mutation. The disease or condition can comprise a muscular dystrophy, an ornithine transcarbamylase deficiency, a retinitis pigmentosa, a breast cancer, an ovarian cancer, Alzheimer's disease, pain, Stargardt macular dystrophy, Charcot-Marie-Tooth disease, Rett syndrome, or any combination thereof. In some cases, an engineered polynucleotide can correct a missense mutation in a patient with Rett (e.g. mutate a stop codon to encode for a Trp). In some cases, an engineered polynucleotide can correct a missense mutation or induce a knockdown in a patient with Parkinson's. In some cases, an engineered polynucleotide can induce a mutation in a patient with Alzheimer's, which can reduce cleavage by a protein at a cleavage site in APP. In some cases, an engineered polynucleotide can generate exon skipping in a patient with muscular dystrophy. In some cases, an engineered polynucleotide can correct a mutation in HexA in a patient with Tay-Sachs disease. In some cases, an engineered polynucleotide can correct a mutation in HexA in a patient with Tay-Sachs disease. In some cases, an engineered polynucleotide can correct a mutation in a patient with AAT deficiency (e.g. edit SERPINA1). In some cases, Administration of a composition can be sufficient to: (a) decrease expression of a gene relative to an expression of the gene prior to administration; (b) edit at least one point mutation in a subject, such as a subject in need thereof; (c) edit at least one stop codon in the subject to produce a readthrough of a stop codon; (d) produce an exon skip in the subject, or (e) any combination thereof. A disease or condition can comprise a muscular dystrophy. A muscular dystrophy can include myotonic, Duchenne, Becker, Limb-girdle, facioscapulohumeral, congenital, oculopharyngeal, distal, Emery-Dreifuss, or any combination thereof. A disease or condition can comprise pain, such as a chronic pain. Pain can include neuropathic pain, nociceptive pain, or a combination thereof. Nociceptive pain can include visceral pain, somatic pain, or a combination thereof. The targeting sequence can comprise a sequence with at least 75%, at least 80%, at least 85%, at least 90%, at least 95%, at least 97%, at least 99%, or 100% sequence identity to any one of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

In some embodiments, the engineered polynucleotide is at least 50%, at least 60%, at least 70%, at least 75%, at least 80%, at least 85%, at least 90%, at least 95%, at least 97%, at least 99%, or 100% complementary to a target sequence of SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, or SEQ ID NO: 17.

### RNA Editing

The present disclosure provides compositions of engineered polynucleotides having snRNA sequences and snRNA hairpins (e.g., SmOPT sequences and U7 hairpins) that facilitate RNA editing of a base in a target RNA of interest. For example, engineered polynucleotides of the present disclosure facilitate an RNA edit comprising a chemical modification of a base, such as deamination of an adenosine (A) to an inosine (I). Inosines are read as guanosines (G). As such, the engineered polynucleotides and methods of use thereof disclosed herein of RNA editing can be used for correction of a G to A point mutation in a gene that may be implicated in a disease or disease pathway. Thus, engineered polynucleotides disclosed herein can be used as a therapeutic in a method of treatment.

In some embodiments, the presence of a bulge in a dsRNA substrate may position ADAR to selectively edit the target A in the target RNA and reduce off-target editing of non-target As in the target RNA. In some embodiments, the presence of a bulge in a dsRNA substrate may recruit additional ADAR. Bulges in dsRNA substrates disclosed herein may recruit other proteins, such as other RNA editing entities. In some embodiments, a bulge positioned 5′ of the edit site may facilitate base-flipping of the target A to be edited. A bulge may also help confer sequence specificity. A bulge may help direct ADAR editing by constraining it in an orientation that yield selective editing of the target A.

An RNA editing entity can be any one of: an ADAR protein (such as ADAR1, ADAR2, ADAR3 or any combination thereof), any APOBEC protein (such as APOBEC1, APOBEC2, APOBEC3A, APOBEC3B, APOBEC3C, APOBEC3E, APOBEC3F, APOBEC3G, APOBEC3H, APOBEC4, or any combination thereof), a zinc finger nuclease, a transcription activator-like effector nuclease (TALEN), a meganuclease, or a combination thereof. In some cases, the ADAR or APOBEC protein recruited can be mammalian. In some cases, the ADAR or APOBEC protein recruited can be human. In some cases, the ADAR or APOBEC protein recruited can be recombinant (such as an exogenously delivered ADAR or APOBEC), modified (such as an exogenously delivered ADAR or APOBEC), endogenous (such as an endogenous ADAR or APOBEC), or any combination thereof. In some cases, the RNA editing entity can be a fusion protein, such as any of the RNA editing entities provided herein. In some cases, the RNA editing entity can be a functional portion of an RNA editing entity, such as any of the RNA editing proteins provided herein. Any of the abovementioned RNA editing entities can be adapted for use with a composition and/or method provided herein.

Adenosine Deaminase Acting on RNA (ADAR) and biologically active fragments thereof can be enzymes that catalyze the chemical conversion of adenosines to inosines in double-stranded RNA (dsRNA) substrates. Because the properties of inosine mimic those of guanosine (inosine will form two hydrogen bonds with cytosine, for example), inosine can be recognized as guanosine by the translational cellular machinery. Adenosine-to-inosine (A-to-I) RNA editing, therefore, effectively changes the primary sequence of RNA targets. ADAR enzymes share a common domain architecture comprising a variable number of amino-terminal dsRNA binding domains (dsRBDs) and a carboxy-terminal catalytic deaminase domain. Human ADARs possess two or three dsRBDs.

Three human ADAR genes have been identified (ADARs 1-3) with ADAR1 (official symbol ADAR) and ADAR2 (ADARB1) proteins having well-characterized adenosine deamination activity. ADARs have a typical modular domain organization that includes at least two copies of a dsRNA binding domain (dsRBD; ADAR1 with three dsRBDs; ADAR2 and ADAR3 with two copies) in their N-terminal region followed by a C-terminal deaminase domain.

ADAR1 and ADAR2 are two exemplary species of ADAR that are involved in mRNA editing in vivo. Non-limiting exemplary sequences for ADAR1 may be found under the following reference numbers: HGNC: 225; Entrez Gene: 103; Ensembl: ENSG 00000160710; OMIM: 146920; UniProtKB: P55265; and GeneCards: GC01M154554, as well as biological equivalents thereof. Non-limiting exemplary sequences for ADAR2 may be found under the following reference numbers: HGNC: 226; Entrez Gene: 104; Ensembl: ENSG00000197381; OMIM: 601218; UniProtKB: P78563; and GeneCards: GC21P045073, as well as biological equivalents thereof. Biologically active fragments of ADAR are also provided herein and can be included when referring to an ADAR.

The present disclosure contemplates use of interferon α as a means of increasing endogenous ADAR1 expression. Commercial sources of isolated or recombinant interferon α include but are not limited to Sigma-Aldrich, R&D Systems, Abcam, and Thermo Fisher Scientific. Alternatively, interferon α may be produced using a known vector and given protein sequence, e.g., Q6QNB6 (human IFNA).

APOBEC and biologically active fragments thereof can refer to any protein, such as an exemplary RNA editing entity that falls within the family of evolutionarily conserved cytidine deaminases involved in mRNA editing—catalyzing a C to U conversion—and equivalents thereof. In some respects, the term APOBEC refers to any one of APOBEC1, APOBEC2, APOBEC3A, APOBEC3B, APOBEC3C, APOBEC3E, APOBEC3F, APOBEC3G, APOBEC3H, APOBEC4, or equivalents each thereof. Non-limiting exemplary sequences of fusion proteins comprising one or more APOBEC domains are provided herein both fused to an ADAR domain or fused to alternative domains to render them suitable for use in an RNA editing system. To this end, APOBECs can be considered an equivalent of ADAR—catalyzing editing albeit by a different conversion. Thus, not to be bound by theory, the embodiments contemplated herein for use with an ADAR based editing system can be adapted for use in an APOBEC based RNA editing system.

In some cases, an RNA editing entity does not form a second structure comprising a stem-loop. In some cases, at least a portion of the RNA editing entity forms a second structure comprising a stem-loop. In some cases, the portion of the RNA editing entity forms a second structure that does not comprise a stem-loop. In some cases, the portion of the RNA editing entity forms a secondary structure comprising a linear portion. In some cases, the portion of the RNA editing entity forms a secondary structure comprising a cruciform or portion thereof.

In some embodiments, the editing of the RNA affects gene expression. In some embodiments, the RNA becomes destabilized. In some embodiments, the gene is not expressed. In some embodiments, alternative splicing is affected. In some embodiments, a different isoform of the gene is expressed.

Recruitment of an ADAR protein to the target sequence may involve contacting the RNA duplex composed of the engineered polynucleotide and the target sequence, in which the targeting sequence is not 100% complementary to the targeting sequence, therefore at least one. In some embodiments, the targeting sequence when bound to the pre-mRNA comprises at least one nucleotide that is not complementary to the pre-mRNA, producing a bulge. In some embodiments, at least 1, at least 2, at least 3, at least 4, at least 5 nucleotides are not complementary to the pre-mRNA, producing bulges.

An engineered polynucleotide having snRNA sequences and snRNA hairpins (e.g., SmOPT sequences and U7 hairpins or variants thereof) can be configured to promote an edit in the pre-mRNA when associated with the pre-mRNA in the presence of the deaminase, thereby forming a deaminase recruiting domain. In some instances, an engineered polynucleotide as described herein having snRNA sequences and snRNA hairpins can comprises a non-complementary nucleotide that can improve affinity for a deaminase with the target mRNA. The deaminase can facilitate a chemical modification of a base of a polynucleotide of the pre-mRNA. In some cases, at least one mismatch produced by at least one nucleotide that is not complementary to the pre-mRNA at least partially associates with the deaminase.

In some cases, the deaminase recruiting domain can comprise at least one stem loop. The stem loop can be either right or left-handed. The stem loop can comprise at least 75%, at least 80% m at least 85%, at least 90%, at least 95%, at least 96%, at least 97%, at least 98%, or at least 99% sequence identity to a GluR2 domain.

The one non-complementary nucleotide to the pre-mRNA within the region targeting the pre-mRNA is located at 45, 46, 47, 48, 49, 50, 51, 52, 53, 54, or 55 bases from either end of the targeting sequence.

The edit can be configured to occur within the pre-mRNA. The edit can be configured to occur in an intro or exon. The edit is configured to occur within the splice signal. The edit can be configured to occur in an untranslated region. The edit can be configured to promote skipping of an exon.

The activity of RNA editing by the engineered polynucleotide can be measured in order to determine the success of a desired activity. In some embodiments, the desired activity is exon skipping.

The editing efficiency of an engineered polynucleotide having snRNA sequences and snRNA hairpins (e.g., SmOPT sequences and U7 hairpins or variants thereof) can be measured by an in vitro assay, such as a droplet digital PCR dropoff assay to detect editing (multiple sequence insertion, deletions, or mutations) with respect to a reference sample. In this assay, at least two probes are used in order to assess a mutation hotspot in a sample. The probes are at least partially complementary to the mutation hotspot. A fluorophore-conjugated dropoff probe can be designed such that it anneals to the unedited target sequence but not to a base-edited target sequence. A second reference probe, conjugated to a different fluorophore, can be designed to anneal next to the dropoff probe target sequence, and detect the target transcript regardless of editing. Primers flanking both probes will amplify the target transcript in a droplet digital PCR reaction. The percent editing can then be reported as transcripts negative for the dropoff probe but positive for the reference probe compared to total transcripts positive for the reference probe.

### Exon Skipping

In some embodiments, the engineered polynucleotides of the present disclosure having snRNA sequences and snRNA hairpins (e.g., SmOPT sequences and U7 hairpins) facilitates an RNA edit (e.g., by ADAR) that results in exon skipping. The engineered polynucleotide of the present disclosure can improve exon skipping as compared to polynucleotide constructs not containing snRNA elements and/or mismatches. The exon may contain a mutation that alters its function. The engineered polynucleotide can have improved exon skipping as compared to polynucleotide constructs not containing snRNA elements and/or mismatches when measured in vitro. Efficiency of exon skipping can be measured by quantitative PCR, droplet digital PCR, or RNA sequencing.

The exon skipping efficiency of the engineered polynucleotide can be measured by an in vitro assay, such as a quantitative PCR assay or droplet digital PCR assay to detect the proportion of exon-skipped transcripts relative to the proportion of unskipped transcripts. In this assay, at least two fluorophore-conjugated probes are used, one which specifically anneals to an exon-skipped transcript and another which specifically anneals to an unskipped transcript. ddPCR amplification was performed.

The engineered polynucleotides of the present disclosure can increase the efficiency of exon skipping by at least 1%, at least 5%, at least 10%, at least 20%, at least 30%, at least 40%, at least 50%, at least 60%, at least 70%, at least 80%, at least 90%, at least 95%, at least 96%, at least 97%, at least 98%, at least 99%, or up to 100%, as measured by an ddPCR. The engineered polynucleotides of the present disclosure can increase the efficiency of exon skipping by about 1% to about 50%. The engineered polynucleotides of the present disclosure can increase the efficiency of exon skipping by at least about 1%. The engineered polynucleotides of the present disclosure can increase the efficiency of exon skipping by at most about 50%. The engineered polynucleotides of the present disclosure can increase the efficiency of exon skipping by about 1% to about 5%, about 1% to about 10%, about 1% to about 20%, about 1% to about 30%, about 1% to about 40%, about 1% to about 50%, about 5% to about 10%, about 5% to about 20%, about 5% to about 30%, about 5% to about 40%, about 5% to about 50%, about 10% to about 20%, about 10% to about 30%, about 10% to about 40%, about 10% to about 50%, about 20% to about 30%, about 20% to about 40%, about 20% to about 50%, about 30% to about 40%, about 30% to about 50%, or about 40% to about 50%.

### Therapeutic Applications

The engineered polynucleotide provided herein having snRNA sequences and snRNA hairpins (e.g., SmOPT sequences and U7 hairpins) can be used as therapeutics. In one aspect herein is a method of treating or preventing a condition comprising administering a therapeutic that facilitates an edit of an RNA. In some embodiments, the edit of the RNA may facilitate correction of a mutation. The mutation may be a missense mutation or a nonsense mutation. In some embodiments, the RNA editing may involve introducing mutations into a target RNA of interest. In some embodiments, the guides of the present disclosure facilitate multiple RNA edits of a target RNA.

APP. In some embodiments, the present disclosure provides compositions and methods of use thereof of guide RNAs having snRNA sequences and snRNA hairpins (e.g., SmOPT sequences and U7 hairpins) that are capable of facilitating RNA editing of an amyloid precursor protein (APP). For example, guide RNAs having snRNA sequences and snRNA hairpins (e.g., SmOPT sequences and U7 hairpins) can facilitate editing of the cleavage site in APP, so that beta/gamma secretases exhibit reduced cleavage of APP or can no longer cut APP and, therefore, reduced levels of Abeta 40/42 or no Abetas can be produced. In some embodiments, a guide RNA of the present disclosure having snRNA sequences and snRNA hairpins (e.g., SmOPT sequences and U7 hairpins) can target any one of or any combination of the following sites in APP for RNA editing: K670E, K670R, K670G, M671V, A673V, A673T, D672G, E682G, H684R, K687R, K687E, or K687G, I712X, or T714X. Said guide RNAs having snRNA sequences and snRNA hairpins (e.g., SmOPT sequences and U7 hairpins) targeting a site in APP can be encoded for by an engineered polynucleotide construct of the present disclosure. Sequences of snRNA sequences and snRNA hairpins in said guide RNAs may have at least 80% sequence identity to any one of SEQ ID NO: 41, SEQ ID NO: 42, SEQ ID NO: 43, SEQ ID NO: 44, SEQ ID NO: 45, or SEQ ID NO: 46. Sequences of snRNA sequences and snRNA hairpins in said guide RNAs may have a sequence of any one of SEQ ID NO: 41, SEQ ID NO: 42, SEQ ID NO: 43, SEQ ID NO: 44, SEQ ID NO: 45, or SEQ ID NO: 46. Any promoter (e.g., U1, U6, or U7) disclosed herein may be incorporated to drive expression of said guide RNAs. Said engineered polynucleotides may be delivered via viral vector (e.g., encoded for and delivered via AAV) as disclosed herein and may be administered via any route of administration disclosed herein to a subject in need thereof. The subject may be human and may be at risk of developing or has developed Alzheimer's disease. The subject may be human and may be at risk of developing or has developed a neurological disease in which APP impacts disease pathology. Thus, the guide RNAs of the present disclosure having snRNA sequences and snRNA hairpins (e.g., SmOPT sequences and U7 hairpins) may be used in a method of treatment of neurological diseases (e.g., Alzheimer's disease).

### Alpha-Synuclein (SNCA)

The Alpha-synuclein gene is made up of 5 exons and encodes a 140 amino-acid protein with a predicted molecular mass of −14.5 kDa. The encoded product is an intrinsically disordered protein with unknown functions. Usually, Alpha-synuclein is a monomer. Under certain stress conditions or other unknown causes, α-synuclein self-aggregates into oligomers. Lewy-related pathology (LRP), primarily comprised of Alpha-synuclein in more than 50% of autopsy-confirmed Alzheimer's disease patients' brains. While the molecular mechanism of how Alpha-synuclein affects the development of Alzheimer's disease is unclear, experimental evidence has shown that Alpha-synuclein interacts with Tau-p and may seed the intracellular aggregation of Tau-p. Moreover, Alpha-synuclein could regulate the activity of GSK3β, which can mediate Tau-hyperphosphorylation. Alpha-synuclein can also self-assemble into pathogenic aggregates (Lewy bodies). Both Tau and α-synuclein can be released into the extracellular space and spread to other cells. Vascular abnormalities impair the supply of nutrients and removal of metabolic byproducts, cause microinfarcts, and promote the activation of glial cells. Therefore, a multiplex strategy to substantially reduce Tau formation, alpha-synuclein formation, or a combination thereof can be important in effectively treating neurodegenerative diseases.

The domain structure of Alpha-synuclein comprises an N-terminal A2 lipid-binding alpha-helix domain, a Non-amyloid β component (NAC) domain, and a C-terminal acidic domain. The lipid-binding domain consists of five KXKEGV imperfect repeats. The NAC domain consists of a GAV motif with a VGGAVVTGV consensus sequence and three GXXX sub-motifs—where X is any of Gly, Ala, Val, Ile, Leu, Phe, Tyr, Trp, Thr, Ser or Met. The C-terminal acidic domain contains a copper-binding motif with a DPDNEA consensus sequence. Molecularly, Alpha-synuclein is suggested to play a role in neuronal transmission and DNA repair.

In some cases, a region of Alpha-synuclein can be targeted utilizing compositions provided herein. In some cases, a region of the Alpha-synuclein mRNA can be targeted with the engineered polynucleotides disclosed herein for knockdown. In some cases, a region of the exon or intron of the Alpha-synuclein mRNA can be targeted. In some embodiments, a region of the non-coding sequence of the Alpha-synuclein mRNA, such as the 5′UTR and 3′UTR, can be targeted. In other cases, a region of the coding sequence of the Alpha-synuclein mRNA can be targeted. Suitable regions include but are not limited to a N-terminal A2 lipid-binding alpha-helix domain, a Non-amyloid β component (NAC) domain, or a C-terminal acidic domain.

In some aspects, an alpha-synuclein mRNA sequence is targeted. In some cases, any one of the 3,177 residues of the sequence may be targeted utilizing the compositions and methods provided herein. In some cases, a target residue may be located among residues 1-100, 101-200, 201-300, 301-400, 401-500, 501-600, 601-700, 701-800, 801-900, 901-1000, 1001-1100, 1101-1200, 1201-1300, 1301-1400, 1401-1500, 1501-1600, 1601-1700, 1701-1800, 1801-1900, 1901-2000, 2001-2100, 2101-2200, 2201-2300, 2301-2400, 2401-2500, 2501-2600, 2601-2700, 2701-2800, 2801-2900, 2901-3000, 3001-3100, and/or 3101-3177.

In some embodiments, the present disclosure provides compositions and methods of use thereof of guide RNAs having snRNA sequences and snRNA hairpins (e.g., SmOPT sequences and U7 hairpins) that are capable of facilitating RNA editing of SNCA. In some embodiments, a guide RNA of the present disclosure having snRNA sequences and snRNA hairpins (e.g., SmOPT sequences and U7 hairpins) can knock down expression of SNCA, for example, by facilitating editing at a 3′ UTR of an SNCA gene. Said guide RNAs having snRNA sequences and snRNA hairpins (e.g., SmOPT sequences and U7 hairpins) targeting a site in SNCA can be encoded for by an engineered polynucleotide construct of the present disclosure. Sequences of snRNA sequences and snRNA hairpins in said guide RNAs may have at least 80% sequence identity to any one of SEQ ID NO: 41, SEQ ID NO: 42, SEQ ID NO: 43, SEQ ID NO: 44, SEQ ID NO: 45, or SEQ ID NO: 46. Sequences of snRNA sequences and snRNA hairpins in said guide RNAs may have a sequence of any one of SEQ ID NO: 41, SEQ ID NO: 42, SEQ ID NO: 43, SEQ ID NO: 44, SEQ ID NO: 45, or SEQ ID NO: 46. Exemplary sequences of engineered polynucleotides that can be used to target an SNCA gene include SEQ ID NO: 7, and SEQ ID NO: 8, while exemplary targeting sequences that can target an SNCA gene include SEQ ID NO: 15, SEQ ID NO: 16, and SEQ ID NO: 17. Any promoter (e.g., U1, U6, or U7) disclosed herein may be incorporated to drive expression of said guide RNAs. Said engineered polynucleotides may be delivered via viral vector (e.g., encoded for and delivered via AAV) as disclosed herein and may be administered via any route of administration disclosed herein to a subject in need thereof. The subject may be human and may be at risk of developing or has developed Alzheimer's disease or Parkinson's disease. The subject may be human and may be at risk of developing or has developed a neurological disease in which overexpression of SNCA impacts disease pathology. Thus, the guide RNAs of the present disclosure having snRNA sequences and snRNA hairpins (e.g., SmOPT sequences and U7 hairpins) may be used in a method of treatment of neurological diseases (e.g., Alzheimer's disease).

SERPINA1. In some embodiments, the present disclosure provides compositions and methods of use thereof of guide RNAs having snRNA sequences and snRNA hairpins (e.g., SmOPT sequences and U7 hairpins) that are capable of facilitating RNA editing of serpin family A member 1 (SERPINA1). For example, guide RNAs having snRNA sequences and snRNA hairpins (e.g., SmOPT sequences and U7 hairpins) can facilitate correction of a G to A mutation at nucleotide position 9989 of a SERPINA1 gene. In some embodiments, a guide RNA of the present disclosure having snRNA sequences and snRNA hairpins (e.g., SmOPT sequences and U7 hairpins) can target, for example, E342 of SERPINA1. Said guide RNAs having snRNA sequences and snRNA hairpins (e.g., SmOPT sequences and U7 hairpins) targeting a site in SERPINA1 can be encoded for by an engineered polynucleotide construct of the present disclosure. Sequences of snRNA sequences and snRNA hairpins in said guide RNAs may have at least 80% sequence identity to any one of SEQ ID NO: 41, SEQ ID NO: 42, SEQ ID NO: 43, SEQ ID NO: 44, SEQ ID NO: 45, or SEQ ID NO: 46. Sequences of snRNA sequences and snRNA hairpins in said guide RNAs may have a sequence of any one of SEQ ID NO: 41, SEQ ID NO: 42, SEQ ID NO: 43, SEQ ID NO: 44, SEQ ID NO: 45, or SEQ ID NO: 46. Exemplary targeting sequences that can target a SERPINA1 gene include SEQ ID NO: 26 or SEQ ID NO: 27. Any promoter (e.g., U1, U6, or U7) disclosed herein may be incorporated to drive expression of said guide RNAs. Said engineered polynucleotides may be delivered via viral vector (e.g., encoded for and delivered via AAV) as disclosed herein and may be administered via any route of administration disclosed herein to a subject in need thereof. The subject may be human and may be at risk of developing or has developed alpha-1 antitrypsin deficiency. Such alpha-1 antitrypsin deficiency may be at least partially caused by a mutation of SERPINA1, for which an engineered polynucleotide sequence described herein can facilitate editing in, thus correcting the mutation in SERPINA1 and reducing the incidence of alpha-1 antitrypsin deficiency in the subject. Thus, the guide RNAs of the present disclosure having snRNA sequences and snRNA hairpins (e.g., SmOPT sequences and U7 hairpins) may be used in a method of treatment of alpha-1 antitrypsin deficiency.

ABCA4. In some embodiments, the present disclosure provides compositions and methods of use thereof of guide RNAs having snRNA sequences and snRNA hairpins (e.g., SmOPT sequences and U7 hairpins) that are capable of facilitating RNA editing of ATP binding cassette subfamily A member 4 (ABCA4). For example, guide RNAs having snRNA sequences and snRNA hairpins (e.g., SmOPT sequences and U7 hairpins) can facilitate correction of a G to A mutation at nucleotide position 5714, 5882, or 6320 of an ABCA4 gene. Said guide RNAs having snRNA sequences and snRNA hairpins (e.g., SmOPT sequences and U7 hairpins) targeting a site in ABCA4 can be encoded for by an engineered polynucleotide construct of the present disclosure. Sequences of snRNA sequences and snRNA hairpins in said guide RNAs may have at least 80% sequence identity to any one of SEQ ID NO: 41, SEQ ID NO: 42, SEQ ID NO: 43, SEQ ID NO: 44, SEQ ID NO: 45, or SEQ ID NO: 46. Sequences of snRNA sequences and snRNA hairpins in said guide RNAs may have a sequence of any one of SEQ ID NO: 41, SEQ ID NO: 42, SEQ ID NO: 43, SEQ ID NO: 44, SEQ ID NO: 45, or SEQ ID NO: 46. Exemplary targeting sequences that can target an ABCA4 gene include SEQ ID NO: 20, SEQ ID NO: 21, and SEQ ID NO: 22. Any promoter (e.g., U1, U6, or U7) disclosed herein may be incorporated to drive expression of said guide RNAs. Said engineered polynucleotides may be delivered via viral vector (e.g., encoded for and delivered via AAV) as disclosed herein and may be administered via any route of administration disclosed herein to a subject in need thereof. The subject may be human and may be at risk of developing or has developed Stargardt macular degeneration (or Stargardt's disease). Such Stargardt macular degeneration may be at least partially caused by a mutation of ABCA4, for which an engineered polynucleotide sequence described herein can facilitate editing in, thus correcting the mutation in ABCA4 and reducing the incidence of Stargardt macular degeneration in the subject. Thus, the guide RNAs of the present disclosure having snRNA sequences and snRNA hairpins (e.g., SmOPT sequences and U7 hairpins) may be used in a method of treatment of Stargardt macular degeneration.

LRRK2. Leucine-rich repeat kinase 2 (LRRK2) has been associated with familial and sporadic cases of Parkinson's Disease and immune-related disorders like Crohn's disease. Its aliases include LRRK2, AURA17, DARDARIN, PARK8, RIPK7, ROCO2, or leucine-rich repeat kinase 2. The LRRK2 gene is made up of 51 exons and encodes a 2527 amino-acid protein with a predicted molecular mass of about 286 kDa. The encoded product is a multi-domain protein with kinase and GTPase activities. LRRK2 can be found in various tissues and organs including but not limited to adrenal, appendix, bone marrow, brain, colon, duodenum, endometrium, esophagus, fat, gall bladder, heart, kidney, liver, lung, lymph node, ovary, pancreas, placenta, prostate, salivary gland, skin, small intestine, spleen, stomach, testis, thyroid, and urinary bladder. LRRK2 can be ubiquitously expressed but is generally more abundant in the brain, kidney, and lung tissue. Cellularly, LRRK2 has been found in astrocytes, endothelial cells, microglia, neurons, and peripheral immune cells.

Over 100 mutations have been identified in LRRK2; six of them—G2019S, R1441C/G/H, Y1699C, and I2020T—have been shown to cause Parkinson's Disease through segregation analysis. G2019S and R1441C are the most common disease-causing mutations in inherited cases. In sporadic cases, these mutations have shown age-dependent penetrance: The percentage of individuals carrying the G2019S mutation that develops the disease jumps from 17% to 85% when the age increases from 50 to 70 years old. In some cases, mutation-carrying individuals never develop the disease.

At its catalytic core, LRRK2 contains the Ras of complex proteins (Roc), C-terminal of ROC (COR), and kinase domains. Multiple protein-protein interaction domains flank this core: an armadillo repeats (ARM) region, an ankyrin repeat (ANK) region, a leucine-rich repeat (LRR) domain are found in the N-terminus joined by a C-terminal WD40 domain. The G2019S mutation is located within the kinase domain. It has been shown to increase the kinase activity; for R1441C/G/H and Y1699C, these mutations can decrease the GTPase activity of the Roc domain. Genome-wide association study has found that common variations in LRRK2 increase the risk of developing sporadic Parkinson's Disease. While some of these variations are nonconservative mutations that affect the protein's binding or catalytic activities, others modulate its expression. These results suggest that specific alleles or haplotypes can regulate LRRK2 expression.

Pro-inflammatory signals upregulate LRRK2 expression in various immune cell types, suggesting that LRRK2 is a critical regulator in the immune response. Studies have found that both systemic and central nervous system (CNS) inflammation are involved in Parkinson's Disease's symptoms. Moreover, LRRK2 mutations associated with Parkinson's Disease modulate its expression levels in response to inflammatory stimuli. Many mutations in LRRK2 are associated with immune-related disorders such as inflammatory bowel disease such as Crohn's Disease. For example, both G2019S and N2081D increase LRRK2's kinase activity and are over-represented in Crohn's Disease patients in specific populations. Because of its critical role in these disorders, LRRK2 is an important therapeutic target for Parkinson's Disease and Crohn's Disease. In particular, many mutations, such as point mutations including G2019S, play roles in developing these diseases, making LRRK2 an attractive for therapeutic strategy such as RNA editing.

In some embodiments, the present disclosure provides compositions and methods of use thereof of guide RNAs having snRNA sequences and snRNA hairpins (e.g., SmOPT sequences and U7 hairpins) that are capable of facilitating RNA editing of LRRK2. In some embodiments, a guide RNA of the present disclosure having snRNA sequences and snRNA hairpins (e.g., SmOPT sequences and U7 hairpins) can target the following mutations in LRRK2: E10L, A30P, S52F, E46K, A53T, L119P, A211V, C228S, E334K, N363S, V366M, A419V, R506Q, N544E, N551K, A716V, M712V, I723V, P755L, R793M, I810V, K871E, Q923H, Q930R, R1067Q, S1096C, Q1111H, I1122V, A1151T, L1165P, I1192V, H1216R, S1228T, P1262A, R1325Q, I1371V, R1398H, T1410M, D1420N, R1441G, R1441H, A1442P, P1446L, V1450I, K1468E, R1483Q, R1514Q, P1542S, V1613A, R1628P, M1646T, S1647T, Y1699C, R1728H, R1728L, L1795F, M1869V, M1869T, L1870F, E1874X, R1941H, Y2006H, I2012T, G2019S, I2020T, T2031S, N2081D, T2141M, R2143H, Y2189C, T2356I, G2385R, V2390M, E2395K, M2397T, L2466H, or Q2490NfsX3. Said guide RNAs having snRNA sequences and snRNA hairpins (e.g., SmOPT sequences and U7 hairpins) targeting a site in LRRK2 can be encoded for by an engineered polynucleotide construct of the present disclosure. Sequences of snRNA sequences and snRNA hairpins in said guide RNAs may have at least 80% sequence identity to any one of SEQ ID NO: 41, SEQ ID NO: 42, SEQ ID NO: 43, SEQ ID NO: 44, SEQ ID NO: 45, or SEQ ID NO: 46. Sequences of snRNA sequences and snRNA hairpins in said guide RNAs may have a sequence of any one of SEQ ID NO: 41, SEQ ID NO: 42, SEQ ID NO: 43, SEQ ID NO: 44, SEQ ID NO: 45, or SEQ ID NO: 46. Exemplary targeting sequences that can target a LRRK2 gene include SEQ ID NO: 18 and SEQ ID NO: 19. Any promoter (e.g., U1, U6, or U7) disclosed herein may be incorporated to drive expression of said guide RNAs. Said engineered polynucleotides may be delivered via viral vector (e.g., encoded for and delivered via AAV) as disclosed herein and may be administered via any route of administration disclosed herein to a subject in need thereof. The subject may be human and may be at risk of developing or has developed a disease or condition associated with mutations in LRRK2 (e.g. diseases of the central nervous system (CNS) or gastrointestinal (GI) tract). For example, such diseases of conditions can include Crohn's disease or Parkinson's disease. Such CNS or GI tract diseases (e.g. Crohn's disease or Parkinson's disease) may be at least partially caused by a mutation of LRRK2, for which an engineered polynucleotide sequence described herein can facilitate editing in, thus correcting the mutation in LRRK2 and reducing the incidence of the CNS or GI tract disease in the subject. Thus, the guide RNAs of the present disclosure having snRNA sequences and snRNA hairpins (e.g., SmOPT sequences and U7 hairpins) may be used in a method of treatment of diseases such as Crohn's disease or Parkinson's disease.

DMD. In some embodiments, the present disclosure provides compositions and methods of use thereof of guide RNAs having snRNA sequences and snRNA hairpins (e.g., SmOPT sequences and U7 hairpins) that are capable of facilitating RNA editing of a Duchenne muscular dystrophy (DMD) gene. In some embodiments, a guide RNA of the present disclosure having snRNA sequences and snRNA hairpins (e.g., SmOPT sequences and U7 hairpins) can target an exon of a DMD gene, such as exon 51, 45, 53, 44, 46, 52, 50, 43, 6, 7, 8, 55, 2, 11, 17, 19, 21, 57, 59, 62, 63, 65, 66, 69, 74 and/or 75 in the DMD gene pre-mRNA that at least in part encodes a dystrophin protein. Said guide RNAs having snRNA sequences and snRNA hairpins (e.g., SmOPT sequences and U7 hairpins) targeting a site in a DMD gene can be encoded for by an engineered polynucleotide construct of the present disclosure. Sequences of snRNA sequences and snRNA hairpins in said guide RNAs may have at least 80% sequence identity to any one of SEQ ID NO: 41, SEQ ID NO: 42, SEQ ID NO: 43, SEQ ID NO: 44, SEQ ID NO: 45, or SEQ ID NO: 46. Sequences of snRNA sequences and snRNA hairpins in said guide RNAs may have a sequence of any one of SEQ ID NO: 41, SEQ ID NO: 42, SEQ ID NO: 43, SEQ ID NO: 44, SEQ ID NO: 45, or SEQ ID NO: 46. Exemplary targeting sequences that can target a DMD gene include SEQ ID NO: 13 and SEQ ID NO: 14. Any promoter (e.g., U1, U6, or U7) disclosed herein may be incorporated to drive expression of said guide RNAs. Said engineered polynucleotides may be delivered via viral vector (e.g., encoded for and delivered via AAV) as disclosed herein and may be administered via any route of administration disclosed herein to a subject in need thereof. The subject may be human and may be at risk of developing or has developed a disease or condition associated with mutations in a DMD gene such as DMD. DMD may be at least partially caused by a mutation of a DMD gene, for which an engineered polynucleotide sequence described herein can facilitate editing in, thus correcting the mutation in DMD gene and reducing the incidence of the DMD in the subject. Thus, the guide RNAs of the present disclosure having snRNA sequences and snRNA hairpins (e.g., SmOPT sequences and U7 hairpins) may be used in a method of treatment of diseases such as DMD.

In some embodiments, guide RNAs may target an exon with a mutation wherein the mutation is an insertion, deletion, missense, or nonsense mutation. The mutation can be present in a gene encoding any target described herein. The edited RNA comprises an edited splice signal, resulting in increased exon skipping compared to treatment without the improved polynucleotide construct. In another aspect herein is a method of treating or preventing a condition comprising administering a therapeutic further comprises an snRNA hairpin (e.g., a U7 hairpin of the present disclosure), an snRNA sequence (e.g., a SmOPT sequence of the present disclosure), or both, that enhances editing of an RNA at least partially encoding a target as described herein, wherein the edited RNA comprises an edited splice signal, resulting in increased exon skipping compared to treatment with a comparable an antisense exon skipping construct without a hairpin.

Methods can include use of the engineered polynucleotide to edit a target sequence, which encodes at least a portion of a polypeptide implicated in a disease. The disease includes, but is not limited to, Duchenne's Muscular Dystrophy (DMD), Rett syndrome, Charcot-Marie-Tooth disease, Parkinson's disease, or any combination thereof. In some cases, the disease or condition is associated with a mutation in a DNA molecule or RNA molecule encoding ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof. In some examples, a protein encoded for by a mutated DNA molecule or RNA molecule encoding ABCA4, AAT, SERPINA1, SERPINA1 E342K, HEXA, LRRK2, SNCA, DMD, APP, Tau, GBA, PINK1, RAB7A, CFTR, ALAS1, ATP7B, ATP7B G1226R, HFE C282Y, LIPA c.894 G>A, PCSK9 start site, or SCNN1A start site, a fragment any of these, or any combination thereof contributes to, at least in part, the pathogenesis or progression of a disease. In some examples, the mutation in the DNA or RNA molecule is relative to an otherwise identical reference DNA or RNA molecule.

### Multiplexing

In some cases, the present disclosure encompasses multiplexed therapy, including targeted multiplexed editing of multiple target RNAs, targeted editing of multiple target sites within a target RNA, targeted editing of RNA and knockdown, or any combination thereof. Accordingly, an engineered polynucleotide having the snRNA sequences and snRNA hairpins (e.g., SmOPT and U7) as described herein can be multiplexed to perform multiplex therapy. Targeting more than one target RNA simultaneously may be important and the combination of Tau knockdown and editing of a cleavage site (e.g., a β-cleavage site) in APP may work in synergy. In some cases, use of mRNA base editing to knockdown (as opposed to just editing the cleavage site) expression of APP can be another approach for decreasing Abeta generation. As the compositions can be applied to gene expression knockdown, they could also include a combination of start-site editing to reduce expression, steric hinderance because the guide could block ribosomal activity, increased degradation of the targeted mRNA, or any combination thereof. The compositions and methods disclosed herein, thus, may suppress expression in an ADAR-dependent and ADAR-independent manner.

### Delivery of Multiple Payloads

In some embodiments, a vector of the present disclosure may be a vector that can contain multiple copies of an engineered guide polynucleotide having the snRNA sequences and snRNA hairpins (e.g., SmOPT and U7) as described herein that target multiple target RNAs. For example, a vector can contain at least 2, 3, 4, 5, 6, 7, 8, 9, or 10 copies of an engineered polynucleotide having the snRNA sequences and snRNA hairpins (e.g., SmOPT and U7) as described herein. In some cases, the vectors can contain copies of engineered polynucleotides having the snRNA sequences and snRNA hairpins (e.g., SmOPT and U7) as described herein that are all the same. In some cases, the vectors can contain copies of engineered polynucleotides having the snRNA sequences and snRNA hairpins (e.g., SmOPT and U7) as described herein that are different. Such vectors can be constructed such that multiple copies are operatively coupled polycistronic to a single promoter as described herein. In some instances, the multiple copies can be independently linked to a promoter as described herein.

### Vectors

The present disclosure also provides for vectors that comprise or encode for the engineered polynucleotides disclosed herein.

The compositions provided herein can be delivered by any suitable means. In some cases, a suitable means comprises a vector. Any vector system can be used utilized, including but not limited to: plasmid vectors, minicircle vectors, linear DNA vectors, doggy bone vectors, retroviral vectors, lentiviral vectors, adenovirus vectors, poxvirus vectors; herpesvirus vectors and adeno-associated virus vectors, a liposome, a nanoparticle, an exosome, an extracellular vesicle, a nanomesh, modified versions thereof, good manufacturing practices versions thereof, chimeras thereof, and any combination thereof. In some cases, a vector can be used to introduce a polynucleotide provided herein. In some cases, the polynucleotide comprises a targeting sequence that hybridizes to a region of an RNA provided herein. In some embodiments, a nanoparticle vector can comprise a polymeric-based nanoparticle, an amino lipid-based nanoparticle, a metallic nanoparticle (such as gold-based nanoparticle), a portion of any of these, or any combination thereof.

Vectors provided herein can be used to deliver polynucleotide compositions provided herein. In some cases, at least about 2, 3, 4, or up to 5 different polynucleotides are delivered using a single vector. In some cases, multiple vectors are delivered. In some cases, multiple vector delivery can be co-current or sequential. In some cases, at least two engineered polynucleotides are delivered in a single vector. In other cases, at least two engineered polynucleotides are delivered on separate vectors. Engineered polynucleotides may also be delivered as naked polynucleotides. Any combination of vector and/or a non-vector approach can be taken.

A vector can be employed to deliver a nucleic acid. A vector can comprise DNA, such as double stranded DNA or single stranded DNA. A vector can comprise RNA. In some cases, the RNA can comprise a base modification. The vector can comprise a recombinant vector. The vector can be a vector that is modified from a naturally occurring vector. The vector can comprise at least a portion of a non-naturally occurring vector. Any vector can be utilized. A viral vector can comprise an adenoviral vector, an adeno-associated viral vector (AAV), a lentiviral vector, a retroviral vector, a portion of any of these, or any combination thereof. In some cases, a vector can comprise an AAV vector. A vector can be modified to include a modified VP1 protein (such as an AAV vector modified to include a VP1 protein). In an aspect an AAV vector is a recombinant AAV (rAAV) vector. rAAVs can be composed of substantially similar capsid sequence and structure as found in wild-type AAVs (wtAAVs). However, rAAVs encapsidate genomes that are substantially devoid of AAV protein-coding sequences and have therapeutic gene expression cassettes, such as subject polynucleotides, designed in their place. In some cases, sequences of viral origin can be the ITRs, which may be needed to guide genome replication and packaging during vector production. Suitable AAV vectors can be selected from any AAV serotype or combination of serotypes. For example, an AAV vector can be any one of: AAV1, AAV2, AAV3, AAV4, AAV5, AAV6, AAV7, AAV8, AAV9, AAV 10, AAV11, AAV12, or any combination thereof. In some cases, a vector is selected based on its natural tropism. In some cases, a vector serotype is selected based on its ability to cross the blood brain barrier. AAV9 and AAV10 have been shown to cross the blood brain barrier to transduce neurons and glia. In an aspect, an AAV vector is AAV2, AAV5, AAV6, AAV8, or AAV9. In some cases, an AAV vector is a chimera of at least two serotypes. In an aspect, an AAV vector is of serotypes AAV2, AAV5, and AAV9. In some cases, a chimeric AAV vector comprises rep and ITR sequences from AAV2 and a cap sequence from AAV5. In some cases, rep, cap, and ITR sequences can be mixed and matched from all the of the different AAV serotypes provided herein.

In some embodiments, the vector is a viral vector. In some embodiments, the viral vector is an AAV vector, and wherein the AAV vector is of a serotype selected from the group comprising: AAV2, AAV5, AAV6, AAV8, AAV9, a portion thereof, a fusion product thereof, and any combination thereof. In some embodiments, the AAV vector comprises rep and ITR sequences from AAV2 and a cap sequence from AAV5. In some embodiments, the AAV vector comprises an ITR sequence that is a self-complementary ITR. In some embodiments, the AAV vector that encodes for the engineered polynucleotide is self-complementary.

In some cases, a suitable AAV vector can be further modified to encompass modifications such as in a capsid or rep protein. Modifications can also include deletions, insertions, mutations, and combinations thereof. In some cases, a modification to a vector is made to reduce immunogenicity to allow for repeated dosing. In some cases, a serotype of a vector that is utilized is changed when repeated dosing is performed to reduce and/or eliminate immunogenicity.

Retroviral vectors are useful as agents to mediate retroviral-mediated gene transfer into eukaryotic cells. Retroviral vectors are generally constructed such that the majority of sequences coding for the structural genes of the virus are deleted and replaced by the gene(s) of interest. Most often, the structural genes (e.g., gag, pol, and env), are removed from the retroviral backbone using genetic engineering techniques known in the art. This may include digestion with the appropriate restriction endonuclease or, in some instances, with Bal 31 exonuclease to generate fragments containing appropriate portions of the packaging signal.

These new genes have been incorporated into the proviral backbone in several general ways. The most straightforward constructions are ones in which the structural genes of the retrovirus are replaced by a single gene which then is transcribed under the control of the viral regulatory sequences within the long terminal repeat (LTR). Retroviral vectors have also been constructed which can introduce more than one gene into target cells. Usually, in such vectors one gene is under the regulatory control of the viral LTR, while the second gene is expressed either off a spliced message or is under the regulation of its own, internal promoter.

In a particular embodiment, the viral vector is an adeno-associated virus (AAV). AAV is a tiny non-enveloped virus having a 25 nm capsid. No disease is known or has been shown to be associated with the wild type virus. AAV has a single-stranded DNA (ssDNA) genome. AAV has been shown to exhibit long-term episomal transgene expression, and AAV has demonstrated excellent transgene expression in the brain, particularly in neurons. Vectors containing as little as 300 base pairs of AAV can be packaged and can integrate. Space for exogenous DNA is limited to about 4.7 kb. An AAV vector can be used to introduce DNA into cells. A variety of nucleic acids have been introduced into different cell types using AAV vectors. There are numerous alternative AAV variants (over 100 have been cloned), and AAV variants have been identified based on desirable characteristics. For example, AAV9 has been shown to efficiently cross the blood-brain barrier. Moreover, the AAV capsid can be genetically engineered to increase transduction efficiency and selectivity, e.g., biotinylated AAV vectors, directed molecular evolution, self-complementary AAV genomes and so on. Modified AAV have also been described, including AAV based on ancestral sequences. Other modified AAVs that have been described include chimeric nanoparticles (ChNPs) that have an AAV core that expresses a transgene that is surrounded by layer(s) of acid labile polymers that have embedded antisense oligonucleotides. The compositions and methods disclosed herein is a platform technology, and as such the composition and methods disclosed herein can be used with all known AAVs, including the modified AAVs described in the literature, such as ChNPs.

In another embodiment, the viral vector is an adenovirus-derived vector. The genome of an adenovirus can be manipulated, such that it encodes and expresses a gene product of interest but is inactivated in terms of its ability to replicate in a normal lytic viral life cycle. Suitable adenoviral vectors derived from the adenovirus strain Ad type 5 d1324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells. Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors. Alphaviruses can also be used. Alphaviruses are enveloped single stranded RNA viruses that have a broad host range, and when used in viral gene therapy protocols alphaviruses can provide high-level transient gene expression. Exemplary alphaviruses include the Semliki Forest virus (SFV), Sindbis virus (SIN) and Venezuelan Equine Encephalitis (VEE) virus, all of which have been genetically engineered to provide efficient replication-deficient and -competent expression vectors. Alphaviruses exhibit significant neurotropism, and so are useful for CNS-related diseases.

A vector can be used to deliver an engineered polynucleotide provided herein and an additional polynucleotide targeting a therapeutic target, such as a second polynucleotide. In some cases, the vector comprises or encodes an additional RNA polynucleotide that associates with a second polynucleotide (e.g. an additional therapeutic target). Such vectors can be used to deliver multiplex therapeutics that simultaneously target multiple therapeutic targets, such as, in the case of Alzheimer' and other neurodegenerative disease, amyloid precursor protein and an additional target implicated in the disease such as a Tau protein (e.g., a microtubule-associated protein Tau (MAPT) encoded from a MAPT gene), or an alpha-synuclein protein. Alternatively, or in addition, the additional target can be a further edit site on the polynucleotide encoding the amyloid precursor protein (e.g., on the same polynucleotide). The vector polynucleotide encoding the engineered polynucleotides and the second vector polynucleotide encoding the additional RNA polynucleotide can be contiguous or not contiguous. When the first and second vector polynucleotides are contiguous with each other, they can be operatively linked to the same promoter sequence.

### Non-Viral Vector Approaches

In some cases, a vector may not be a viral vector. Non-viral methods can comprise naked delivery of compositions comprising polynucleotides and the like. In some cases, modifications provided herein can be incorporated into polynucleotides to increase stability and combat degradation when being delivered as naked polynucleotides. In other cases, a non-viral approach can harness use of nanoparticles, liposomes, and the like.

### Pharmaceutical Compositions

Compositions can include any editing entity described herein. A pharmaceutical composition can comprise a first active ingredient. The first active ingredient can comprise a viral vector as described herein, a non-naturally occurring RNA as described herein, or a nucleic acid as described herein. The pharmaceutical composition can be formulated in unit dose form. The pharmaceutical composition can comprise a pharmaceutically acceptable excipient, diluent, or carrier. The pharmaceutical composition can comprise a second, third, or fourth active ingredient—such as to facilitate enhanced exon skipping of genes implicated in a disease or condition.

A composition described herein can compromise an excipient. An excipient can comprise a cryo-preservative, such as DMSO, glycerol, polyvinylpyrrolidone (PVP), or any combination thereof. An excipient can comprise a cryo-preservative, such as a sucrose, a trehalose, a starch, a salt of any of these, a derivative of any of these, or any combination thereof. An excipient can comprise a pH agent (to minimize oxidation or degradation of a component of the composition), a stabilizing agent (to prevent modification or degradation of a component of the composition), a buffering agent (to enhance temperature stability), a solubilizing agent (to increase protein solubility), or any combination thereof. An excipient can comprise a surfactant, a sugar, an amino acid, an antioxidant, a salt, a non-ionic surfactant, a solubilizer, a triglyceride, an alcohol, or any combination thereof. An excipient can comprise sodium carbonate, acetate, citrate, phosphate, poly-ethylene glycol (PEG), human serum albumin (HSA), sorbitol, sucrose, trehalose, polysorbate 80, sodium phosphate, sucrose, disodium phosphate, mannitol, polysorbate 20, histidine, citrate, albumin, sodium hydroxide, glycine, sodium citrate, trehalose, arginine, sodium acetate, acetate, HCl, disodium edetate, lecithin, glycerine, xanthan rubber, soy isoflavones, polysorbate 80, ethyl alcohol, water, teprenone, or any combination thereof.

Compositions and methods disclosed herein can include targeting via an mRNA base editing approach. These vectors may encode for engineered polynucleotides targeting SNCA or Rab7A (to promote exon skipping of the edited splice signal) and one or more additional engineered polynucleotides or other therapeutic agents disclosed herein targeting one or more additional proteins associated with a neurodegeneration disease or condition.

Compositions can include mRNA base editing to edit a pre-mRNA, ii) edit a splice signal, induce exon skipping, or any combination thereof. Editing can result in exon skipping (such as an exon that can contain the start site).

Compositions of the present disclosure can include an engineered polynucleotide for editing a nucleotide in a target RNA polynucleotide sequence. Compositions can employ editing in an ADAR dependent or ADAR independent manner. Compositions can comprise a recruiting domain that facilitates editing of a target RNA via an RNA editing entity.

Compositions and methods provided herein can utilize pharmaceutical compositions. The compositions described throughout can be formulated into a pharmaceutical and be used to treat a human or mammal, in need thereof, diagnosed with a disease. In some cases, pharmaceutical compositions can be used prophylactically.

The compositions provided herein can be utilized in methods provided herein. Any of the provided compositions provided herein can be utilized in methods provided herein. In some cases, a method comprises at least partially preventing, reducing, ameliorating, and/or treating a disease or condition, or a symptom of a disease or condition. A subject can be a human or non-human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician).

### Administration Routes and Dosing

Compositions described herein can employ an AAV (IV/CNS) vector for delivery to a subject. AAV vector delivery can achieve long-term benefits with single dose and can provide opportunity for multiplexed targeting. Methods can include identifying AAV serotypes that can promote central neuronal tropism and biodistribution with CNS/IV dosing.

In some cases, an administration can refer to methods that can be used to enable delivery of compounds or compositions to the desired site of biological action. Delivery can include direct application to the central nervous system (CNS). Delivery can include one that is permissive to cross the blood brain barrier. Delivery can include direct application to the affect tissue or region of the body. A composition provided herein can be administered by any method. A method of administration can be by inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration. Delivery can include topical administration (such as a lotion, a cream, an ointment) to an external surface of a surface, such as a skin. In some cases, administration is by parenchymal injection, intra-thecal injection, intra-ventricular injection, intra-cisternal injection, intravenous injection, or intranasal administration or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). A medical professional can administer the composition. In some cases, a cosmetic professional can administer the composition.

Administration or application of a composition, including any of the engineered polynucleotides disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 days consecutive or nonconsecutive days. A treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, or from about 29 to about 30 days.

Administration or application of a composition, including any engineered polynucleotide exon skipping constructs disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or more. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

In some cases, an administration of any composition provided herein, including pharmaceutical compositions can be in an effective amount, for example to reduce a symptom of a disease or condition and/or to reduce a disease or condition. An effective amount can be sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions. In the context of an immunogenic composition, in some embodiments the effective amount is the amount sufficient to result in a protective response against a pathogen. In other embodiments, the effective amount of an immunogenic composition is the amount sufficient to result in antibody generation against the antigen. In some embodiments, the effective amount is the amount required to confer passive immunity on a subject in need thereof. With respect to immunogenic compositions, in some embodiments the effective amount will depend on the intended use, the degree of immunogenicity of a particular antigenic compound, and the health/responsiveness of the subject's immune system, in addition to the factors described above.

Administration or application of the compositions disclosed herein, including any of the engineered polynucleotides can be performed at least 1, 2, 3, 4, 5, 6, 7, 8, 9, 10, 11, 12, 13, 14, 15, 16, 17, 18, 19, 20, 21, 22, 23, or 24 times a day. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

A composition of the present disclosure, including any of the engineered polynucleotides can be administered/applied as a single dose or as divided doses. In some cases, the compositions described herein can be administered at a first time point and a second time point. In some cases, a composition can be administered such that a first administration is administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

Vectors of the disclosure can be administered at any suitable dose to a subject. Suitable doses can be at least about 5×10⁷ to 50×10¹³ genome copies/mL. In some cases, suitable doses can be at least about 5×10⁷, 6×10⁷, 7×10⁷, 8×10⁷, 9×10⁷, 10×10⁷, 11×10⁷, 15×10⁷, 20×10⁷, 25×10⁷, 30×10⁷ or 50×10⁷ genome copies/mL. In some embodiments, suitable doses can be about 5×10⁷ to 6×10⁷, 6×10⁷ to 7×10⁷, 7×10⁷ to 8×10⁷, 8×10⁷ to 9×10⁷, 9×10⁷ to 10×10⁷, 10×10⁷ to 11×10⁷, 11×10⁷ to 15×10⁷, 15×10⁷ to 20×10⁷, 20×10⁷ to 25×10⁷, 25×10⁷ to 30×10⁷, 30×10⁷ to 50×10⁷, or 50×10⁷ to 100×10⁷ genome copies/mL. In some cases, suitable doses can be about 5×10⁷ to 10×10⁷, 10×10⁷ to 25×10⁷, or 25×10⁷ to 50×10⁷ genome copies/mL. In some cases, suitable doses can be at least about 5×10⁸, 6×10⁸, 7×10⁸, 8×10⁸, 9×10⁸, 10×10⁸, 11×10⁸, 15×10⁸, 20×10⁸, 25×10⁸, 30×10⁸ or 50×10⁸ genome copies/mL. In some embodiments, suitable doses can be about 5×10⁸ to 6×10⁸, 6×10⁸ to 7×10⁸, 7×10⁸ to 8×10⁸, 8×10⁸ to 9×10⁸, 9×10⁸ to 10×10⁸, 10×10⁸ to 11×10⁸, 11×10⁸ to 15×10⁸, 15×10⁸ to 20×10⁸, 20×10⁸ to 25×10⁸, 25×10⁸ to 30×10⁸, 30×10⁸ to 50×10⁸, or 50×10⁸ to 100×10⁸ genome copies/mL. In some cases, suitable doses can be about 5×10⁸ to 10×10⁸, 10×10⁸ to 25×10⁸, or 25×10⁸ to 50×10⁸ genome copies/mL. In some cases, suitable doses can be at least about 5×10⁹, 6×10⁹, 7×10⁹, 8×10⁹, 9×10⁹, 10×10⁹, 11×10⁹, 15×10⁹, 20×10⁹, 25×10⁹, 30×10⁹ or 50×10⁹ genome copies/mL. In some embodiments, suitable doses can be about 5×10⁹ to 6×10⁹, 6×10⁹ to 7×10⁹, 7×10⁹ to 8×10⁹, 8×10⁹ to 9×10⁹, 9×10⁹ to 10×10⁹, 10×10⁹ to 11×10⁹, 11×10⁹ to 15×10⁹, 15×10⁹ to 20×10⁹, 20×10⁹ to 25×10⁹, 25×10⁹ to 30×10⁹, 30×10⁹ to 50×10⁹, or 50×10⁹ to 100×10⁹ genome copies/mL. In some cases, suitable doses can be about 5×10⁹ to 10×10⁹, 10×10⁹ to 25×10⁹, or 25×10⁹ to 50×10⁹ genome copies/mL. In some cases, suitable doses can be at least about 5×10¹⁰, 6×10¹⁰, 7×10¹⁰, 8×10¹⁰, 9×10¹⁰, 10×10¹⁰, 11×10¹⁰, 15×10¹⁰, 20×10¹⁰, 25×10¹⁰, 30×10¹⁰ or 50×10¹⁰ genome copies/mL. In some embodiments, suitable doses can be about 5×10¹⁰ to 6×10¹⁰, 6×10¹⁰ to 7×10¹⁰, 7×10¹⁰ to 8×10¹⁰, 8×10¹⁰ to 9×10¹⁰, 9×10¹⁰ to 10×10¹⁰, 10×10¹⁰ to 11×10¹⁰, 10×10¹⁰ to 15×10¹⁰, 15×10¹⁰ to 20×10¹⁰, 20×10¹⁰ to 25×10¹⁰, 25×10¹⁰ to 30×10¹⁰, 30×10¹⁰ to 50×10¹⁰, or 50×10¹⁰ to 100×10¹⁰ genome copies/mL. In some cases, suitable doses can be about 5×10¹⁰ to 10×10¹⁰, 10×10¹⁰ to 25×10¹⁰, or 25×10¹⁰ to 50×10¹⁰ genome copies/mL. In some cases, suitable doses can be at least about 5×10¹¹, 6×10¹¹, 7×10¹¹, 8×10¹¹, 9×10¹¹, 10×10¹¹, 11×10¹¹, 15×10¹¹, 20×10¹¹, 25×10¹¹, 30×10¹¹ or 50×10¹¹ genome copies/mL. In some embodiments, suitable doses can be about 5×10¹¹ to 6×10¹¹, 6×10¹¹ to 7×10¹¹, 7×10¹¹ to 8×10¹¹, 8×10¹¹ to 9×10¹¹, 9×10¹¹ to 10×10¹¹, 10×10¹¹ to 11×10¹¹, 11×10¹¹ to 15×10¹¹, 15×10¹¹ to 20×10¹¹, 20×10¹¹ to 25×10¹¹, 25×10¹¹ to 30×10¹¹, 30×10¹¹ to 50×10¹¹, or 50×10¹¹ to 100×10¹¹ genome copies/mL. In some cases, suitable doses can be about 5×10¹¹ to 10×10¹¹, 10×10¹¹ to 25×10¹¹, or 25×10¹¹ to 50×10¹¹ genome copies/mL. In some cases, suitable doses can be at least about 5×10¹², 6×10¹², 7×10¹², 8×10¹², 9×10¹², 10×10¹², 11×10¹², 15×10¹², 20×10¹², 25×10¹², 30×10¹² or 50×10¹² genome copies/mL. In some embodiments, suitable doses can be about 5×10¹² to 6×10¹², 6×10¹² to 7×10¹², 7×10¹² to 8×10¹², 8×10¹² to 9×10¹², 9×10¹² to 10×10¹², 10×10¹² to 11×10¹², 11×10¹² to 15×10¹², 15×10¹² to 20×10¹², 20×10¹² to 25×10¹², 25×10¹² to 30×10¹², 30×10¹² to 50×10¹², or 50×10¹² to 100×10¹² genome copies/mL. In some cases, suitable doses can be about 5×10¹² to 10×10¹², 10×10¹² to 25×10¹², or 25×10¹² to 50×10¹² genome copies/mL. In some cases, suitable doses can be at least about 5×10¹³, 6×10¹³, 7×10¹³, 8×10¹³, 9×10¹³, 10×10¹³, 11×10¹³, 15×10¹³, 20×10¹³, 25×10¹³, 30×10¹³ or 50×10¹³ genome copies/mL. In some embodiments, suitable doses can be about 5×10¹³ to 6×10¹³, 6×10¹³ to 7×10¹³, 7×10¹³ to 8×10¹³, 8×10¹³ to 9×10¹³, 9×10¹³ to 10×10¹³, 10×10¹³ to 11×10¹³, 11×10¹³ to 15×10¹³, 15×10¹³ to 20×10¹³, 20×10¹³ to 25×10¹³, 25×10¹³ to 30×10¹³, 30×10¹³ to 50×10¹³, or 50×10¹³ to 100×10¹³ genome copies/mL. In some cases, suitable doses can be about 5×10¹³ to 10×10¹³, 10×10¹³ to 25×10¹³, or 25×10¹³ to 50×10¹³ genome copies/mL. In some cases, suitable doses can be at least about 5×10¹³, 6×10¹³, 7×10¹³, 8×10¹³, 9×10¹³, 10×10¹³, 11×10¹³, 15×10¹³, 20×10¹³, 25×10¹³, 30×10¹³ or 50×10¹³ genome copies/mL. In some embodiments, suitable doses can be about 5×10¹³ to 6×10¹³, 6×10¹³ to 7×10¹³, 7×10¹³ to 8×10¹³, 8×10¹³ to 9×10¹³, 9×10¹³ to 10×10¹³, 10×10¹³ to 11×10¹³, 11×10¹³ to 15×10¹³, 15×10¹³ to 20×10¹³, 20×10¹³ to 25×10¹³, 25×10¹³ to 30×10¹³, 30×10¹³ to 50×10¹³, or 50×10¹³ to 100×10¹³ genome copies/mL. In some cases, suitable doses can be about 5×10¹³ to 10×10¹³, 10×10¹³ to 25×10¹³, or 25×10¹³ to 50×10¹³ genome copies/mL.

In some cases, the dose of virus particles administered to the individual can be any at least about 1×10⁷ to about 1×10¹³ genome copies/kg body weight. In some embodiments, the dose of virus particles administered to the individual can be 1×10⁷, 2×10⁷, 3×10⁷, 4×10⁷, 5×10⁷, 6×10⁷, 7×10⁷, 8×10⁷, or 9×10⁷ genome copies/kg body weight. In some embodiments, the dose of virus particles administered to the individual can be 1×10⁸, 2×10⁸, 3×10⁸, 4×10⁸, 5×10⁸, 6×10⁸, 7×10⁸, 8×10⁸, or 9×10⁸ genome copies/kg body weight. In some embodiments, the dose of virus particles administered to the individual can be 1×10⁹, 2×10⁹, 3×10⁹, 4×10⁹, 5×10⁹, 6×10⁹, 7×10⁹, 8×10⁹, or 9×10⁹ genome copies/kg body weight. In some embodiments, the dose of virus particles administered to the individual can be 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ genome copies/kg body weight. In some embodiments, the dose of virus particles administered to the individual can be 1×10¹¹, 2×10¹¹, 3×10¹¹, 4×10¹¹, 5×10¹¹, 6×10¹¹, 7×10¹¹, 8×10¹¹, or 9×10¹¹ genome copies/kg body weight. In some embodiments, the dose of virus particles administered to the individual can be 1×10¹², 4×10¹², 5×10¹², 6×10¹², 7×10¹², 8×10¹², or 9×10¹² genome copies/kg body weight. In some embodiments, the dose of virus particles administered to the individual can be 1×10¹³, 2×10¹³, 3×10¹³, 4×10¹³, 5×10¹³, 6×10¹³, 7×10¹³, 8×10¹³, or 9×10¹³ genome copies/kg body weight.

### Kits

Also disclosed herein is a kit comprising, or alternatively consisting essentially of, or yet further consisting of the engineered polynucleotide of this disclosure, the isolated polynucleotide encoding the engineered polynucleotide of this disclosure, the vector expressing the engineered polynucleotide of this disclosure, the recombinant cell expressing the engineered polynucleotide of this disclosure, or the compositions disclosed herein and instructions for use. In one aspect, the instructions recite the methods of using the engineered polynucleotide disclosed herein.

A kit may comprise a viral vector. The viral vector may be packaged in a container. The kit may comprise a non-naturally occurring RNA. The non-naturally occurring RNA may be packaged in a container. The kit may comprise a syringe. The kit may comprise a pharmaceutical composition as described herein. The kit may comprise instructions for administration of a viral vector, a non-naturally occurring RNA, a pharmaceutical composition as described herein.

### Numbered Embodiments

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

Embodiment 1. An engineered polynucleotide comprising: a targeting sequence that at least partially hybridizes to at least a portion of a target RNA and contains at least one mismatch when at least partially hybridized to the portion of the target RNA; an Sm or Sm-like protein binding domain, or variant thereof, from a spliceosomal snRNA or a non-spliceosomal small nuclear RNA (snRNA); a hairpin from a spliceosomal snRNA or a non-spliceosomal snRNA, or a variant of either of these; wherein the engineered polynucleotide is configured to facilitate editing of a base of the target RNA by an RNA editing entity.

Embodiment 2. An engineered polynucleotide comprising: a targeting sequence that at least partially hybridizes to at least a portion of a target RNA and contains at least one mismatched nucleotide, wherein the target RNA comprises a mutation in an exon that is implicated in a disease or condition; an Sm or Sm-like protein binding domain or variant thereof from a spliceosomal snRNA or a non-spliceosomal small nuclear RNA (snRNA); and a hairpin from a spliceosomal snRNA, a non-spliceosomal snRNA, or a variant of either of these; wherein the engineered polynucleotide is configured to facilitate exon skipping of the exon in the target RNA.

Embodiment 3. The engineered polynucleotide of embodiment 1 or 2, wherein the mismatch comprises at least one adenine-guanine (A-G) mismatch, at least one adenine-adenine (A-A) mismatch, or at least one adenine-cytosine (A-C).

Embodiment 4. The engineered polynucleotide of embodiment 3, wherein the mismatch comprises an A-C mismatch.

Embodiment 5. The engineered polynucleotide of embodiment 4, wherein the A-C mismatch is configured to promote an edit in the target RNA by a deaminase when associated with the target RNA in the presence of the deaminase.

Embodiment 6. The engineered polynucleotide of any one of embodiments 1-5, wherein the Sm or Sm-like protein binding domain or variant thereof and the hairpin are on a 3′ end of the engineered polynucleotide.

Embodiment 7. The engineered polynucleotide of any one of embodiments 1-6, wherein the engineered polynucleotide comprises a plurality of Sm or Sm-like protein binding domains or variants thereof and a plurality of hairpins.

Embodiment 8. The engineered polynucleotide of any one of embodiments 1-7, wherein the targeting sequence is from about 25 bases to about 200 bases in length.

Embodiment 9. The engineered polynucleotide of any one of embodiments 1-7, wherein the targeting sequence is at least about 30 bases in length.

Embodiment 10. The engineered polynucleotide of any one of embodiments 1-9, wherein the engineered polynucleotide is operably linked to an RNA polymerase II-type promoter.

Embodiment 11. The engineered polynucleotide of embodiment 10, wherein the RNA polymerase II-type promoter comprises a U1 promoter.

Embodiment 12. The engineered polynucleotide of embodiment 10, wherein the RNA polymerase II-type promoter comprises a U7 promoter.

Embodiment 13. The engineered polynucleotide of any one of embodiments 1-12, wherein the engineered polynucleotide is operably linked to a U6 promoter.

Embodiment 14. The engineered polynucleotide of any one of embodiments 1-13, wherein Sm or Sm-like protein binding domain, or variant thereof is a SmOPT sequence.

Embodiment 15. The engineered polynucleotide of embodiment 14, wherein the SmOPT sequence comprises a sequence with at least about 80% sequence identity to AAUUUUUGG or SEQ ID NO: 41.

Embodiment 16. The engineered polynucleotide of embodiment 14, wherein the SmOPT sequence comprises AAUUUUUGG or SEQ ID NO: 41.

Embodiment 17. The engineered polynucleotide of any one of embodiments 1-16, wherein the hairpin is from a mouse U7 snRNA, a human U7 snRNA, or a human U1 snRNA.

Embodiment 18. The engineered polynucleotide of any one of embodiments 1-17, wherein the hairpin is a chimeric hairpin of one or more of a mouse U7 snRNA, a human U7 snRNA, a human U1 snRNA.

Embodiment 19. The engineered polynucleotide of any one of embodiments 1-18, wherein the hairpin comprises a sequence that has at least 80%, at least 85%, at least 90%, at least 92%, at least 95%, at least 97%, or at least 99% sequence identity to the hairpin sequence of any one of SEQ ID NO: 42, SEQ ID NO: 43, SEQ ID NO: 45, or SEQ ID NO: 46.

Embodiment 20. The engineered polynucleotide of any one of embodiments 19, wherein the hairpin comprises the hairpin sequence of any one of SEQ ID NO: 42, SEQ ID NO: 43, SEQ ID NO: 45, or SEQ ID NO: 46.

Embodiment 21. The engineered polynucleotide of any one of embodiments 1-20, wherein the hairpin comprises the hairpin sequence of SEQ ID NO: 43.

Embodiment 22. The engineered polynucleotide of any one of embodiments 1-21, further comprising a U7 box terminator at the 3′ end of the engineered polynucleotide.

Embodiment 23. The engineered polynucleotide of any one of embodiments 1-22, wherein the targeting sequence from 5′ to 3′ comprises the targeting sequence, the Sm or Sm-like protein binding domain or variant thereof, and the hairpin.

Embodiment 24. The engineered polynucleotide of embodiment 2, wherein the engineered polynucleotide is configured to facilitate editing of a base of a nucleotide of the target RNA by an RNA editing entity.

Embodiment 25. The engineered polynucleotide of any one of embodiments 1-24, wherein the RNA editing entity comprises an ADAR protein, an APOBEC protein, or both.

Embodiment 26. The engineered polynucleotide of any one of embodiments 1-24, wherein the RNA editing entity comprises ADAR and wherein the ADAR comprises ADAR1 or ADAR2.

Embodiment 27. The engineered polynucleotide of any one of embodiments 1-26, wherein the targeting sequence at least partially binds to a target RNA that is implemented in a disease or condition.

Embodiment 28. The engineered polynucleotide of embodiment 27, wherein the target RNA is selected from the group consisting of RAB7A, ABCA4, SERPINA1, HEXA, LRRK2, SNCA, DMD, APP, Tau, CFTR, ALAS1, ATP7B, HFE, LIPA, PCSK9 start site, or SCNN1A start site, a fragment any of these, and any combination thereof.

Embodiment 29. The engineered polynucleotide of embodiment 28, wherein the target RNA is SERPINA1, and wherein the SERPINA1 comprises an E342K mutation.

Embodiment 30. The engineered polynucleotide of embodiment 28, wherein the target RNA is LRRK2, and wherein the LRRK2 comprises an G2019S mutation.

Embodiment 31. The engineered polynucleotide of any one of embodiments 1-30, wherein the disease or condition comprises Rett syndrome, Huntington's disease, Parkinson's Disease, Alzheimer's disease, a muscular dystrophy, or Tay-Sachs Disease.

Embodiment 32. The engineered polynucleotide of any one of embodiments 1-31, wherein the engineered polynucleotide further comprises an additional sequence from an snRNA.

Embodiment 33. The engineered polynucleotide of embodiments 1-32, wherein the snRNA sequence comprises at least in part a U1, U2, U4, U5, U6, or U7 snRNA sequence.

Embodiment 34. The engineered polynucleotide of any one of embodiments 1-33, wherein the engineered polynucleotide comprises a sequence that has at least 80% identity to any one of SEQ ID NO: 1-SEQ ID NO: 33 or a variant thereof.

Embodiment 35. The engineered polynucleotide of embodiment 34, wherein the snRNA sequence comprises a sequence that has at least 90% identity to any one of SEQ ID NO: 1-SEQ ID NO: 33 or a variant thereof.

Embodiment 36. The engineered polynucleotide of embodiment 4, wherein the snRNA promoter comprises at least in part a U1, U2, U4, U5, U6, or U7 snRNA promoter.

Embodiment 37. The engineered polynucleotide of any one of embodiments 1-36, wherein the targeting sequence is at least partially complementary to a splice signal proximal to an exon within the target RNA.

Embodiment 38. The engineered polynucleotide of embodiment 37, wherein the targeting sequence is:
(a) at least partially complementary to a branch point upstream of an exon within the target RNA; or
(b) the targeting sequence is at least partially complementary to a donor splice site downstream of an exon within the target RNA.

Embodiment 39. The engineered polynucleotide of embodiment 1, wherein the engineered polynucleotide has improved efficiency of exon skipping as compared to a comparable exon skipping construct without the Sm or Sm-like binding domain or variant thereof, without the hairpin, or without the Sm or Sm-like binding domain or variant thereof and the hairpin when measured in vitro.

Embodiment 40. The engineered polynucleotide of embodiment 39, wherein the efficiency is determined performing a digital droplet PCR dropoff assay to detect a percent skipping of the exon skipped by the engineered polynucleotide in a cell transfected by the engineered polynucleotide, relative to a cell comprising a comparable exon skipping construct without the at least one mismatched nucleotide.

Embodiment 41. The engineered polynucleotide of any one of embodiments 37-40, wherein the engineered polynucleotide is configured to facilitate an edit of a base within the splice signal.

Embodiment 42. The engineered polynucleotide of embodiment 41, wherein the edit is configured to promote at least in part skipping of an exon.

Embodiment 43. The engineered polynucleotide of any one of embodiments 39-42, wherein the engineered polynucleotide has increased efficiency of exon skipping of at least 1%, at least 5%, at least 10%, at least 20%, at least 30%, at least 40%, or at least 50% as measured by an in vitro assay.

Embodiment 44. The engineered polynucleotide of any one of embodiments 1-43, wherein the at least one mismatched nucleotide at least in part configures the engineered polynucleotide, when associated with the target RNA, to facilitate an edit of base of the target RNA via a deaminase.

Embodiment 45. The engineered polynucleotide of embodiment 44, wherein the targeting sequence when bound to the target RNA and in association with a deaminase facilitates a chemical modification of a base of a polynucleotide of the target RNA by the deaminase.

Embodiment 46. The engineered polynucleotide of any one of embodiments 1-45, wherein the mismatch is located from about 1 to about 200 bases from either end of the targeting sequence.

Embodiment 47. The engineered polynucleotide of any one of embodiments 1-45, wherein the mismatch is located at least 45, 46, 47, 48, 49, 50, 51, 52, 53, 54, or 55 bases from either end of the targeting sequence.

Embodiment 48. The engineered polynucleotide of any one of embodiments 1-47, further comprising a deaminase recruiting domain.

Embodiment 49. The engineered polynucleotide of embodiment 47, where the deaminase recruiting domain is selected from the group consisting of: GluR2, Alu, a portion of either of these, a variant of either of these, and any combination thereof.

Embodiment 50. The engineered polynucleotide of embodiment 47 or 48, wherein the deaminase recruiting domain comprises a stem loop.

Embodiment 51. The engineered polynucleotide of embodiment 49, wherein the stem loop is a left-handed stem loop or a right-handed stem loop.

Embodiment 52. The engineered polynucleotide of embodiment 50, wherein the stem loop comprises at least about 80% sequence identity to a GluR2 domain.

Embodiment 53. The engineered polynucleotide of any one of embodiments 1-51, wherein the engineered polynucleotide comprises at least one chemically modified nucleotide or nucleoside.

Embodiment 54. The engineered polynucleotide of any one of embodiments 1-52, wherein the at least one chemical modification comprises a modification of one or both of non-linking phosphate oxygen atoms in a phosphodiester backbone linkage of the engineered polynucleotide as provided in Table 1.

Embodiment 55. The engineered polynucleotide of any one of embodiments 1-53, wherein the engineered polynucleotide, when present in an aqueous solution and not bound to the target RNA, lacks at least one of a bulge, a polynucleotide loop, a structured domain, or any combination thereof.

Embodiment 56. The engineered polynucleotide of any one of embodiments 1-54, wherein the engineered polynucleotide, when at least partially hybridized to the target RNA, comprises a bulge, an internal loop, a hairpin, or any combination thereof.

Embodiment 57. The engineered polynucleotide of any one of embodiments 1-55, wherein the engineered polynucleotide, when at least partially hybridized to the target RNA, comprises the bulge.

Embodiment 58. The engineered polynucleotide of embodiment 56, wherein the bulge is an asymmetric bulge.

Embodiment 59. The engineered polynucleotide of embodiment 56, wherein the bulge is a symmetric bulge.

Embodiment 60. The engineered polynucleotide of any one of embodiments 55-58, wherein the engineered polynucleotide, when at least partially hybridized to the target RNA, comprises the internal loop.

Embodiment 61. The engineered polynucleotide of embodiment 59, wherein the internal loop is an asymmetric loop.

Embodiment 62. The engineered polynucleotide of embodiment 59, wherein the internal loop is a symmetric loop.

Embodiment 63. The engineered polynucleotide of any one of embodiments 1-53, wherein the engineered polynucleotide comprises a structural loop stabilized scaffold.

Embodiment 64. The engineered polynucleotide of embodiment 62, wherein the structural loop stabilized scaffold further comprises a targeting sequence that, when at least partially hybridized to the target RNA, facilitates a chemical modification of the base of the nucleotide of the target RNA via an RNA editing enzyme; and wherein the targeting sequence comprises at least about 4 contiguous nucleotides.

Embodiment 65. The engineered polynucleotide of any one of embodiments 1-63, wherein the engineered polynucleotide further comprises a first spacer domain and a second spacer domain flanking the targeting sequence, wherein the engineered polynucleotide is configured to undergo circularization after transcription in a mammalian cell.

Embodiment 66. The engineered polynucleotide of embodiment 64, wherein the engineered polynucleotide comprises a ribozyme domain 5′ to the first spacer domain, 3′ to the second spacer domain, or both.

Embodiment 67. The engineered polynucleotide of embodiment 65, wherein the engineered polynucleotide comprises a ligation domain between the ribozyme domain and the first spacer domain or between the ribozyme domain and the second spacer domain.

Embodiment 68. The engineered polynucleotide of any one of embodiments 1-66, wherein the engineered polynucleotide is linear when the polynucleotide sequence is represented 2-dimensionally.

Embodiment 69. The engineered polynucleotide of any one of embodiments 1-66, wherein the engineered polynucleotide lacks a 3′ reducing hydroxyl exposed to solvent.

Embodiment 70. The engineered polynucleotide of any one of embodiments 1-66, wherein the engineered polynucleotide is configured to undergo a chemical change when transformed into a mammalian cell, such that after the chemical change, the engineered chemically modified lacks a 3′ reducing hydroxyl exposed to solvent.

Embodiment 71. The engineered polynucleotide of any one of embodiments 64-69, wherein the targeting sequence does not comprise an aptamer.

Embodiment 72. The engineered polynucleotide of any one of embodiments 1-66, wherein the engineered polynucleotide does not comprise or encode a sequence configured for RNA interference (RNAi).

Embodiment 73. The engineered polynucleotide of any one of embodiments 1-72, wherein the targeting sequence is configured to at least partially associate with at least a portion of a 3′ or 5′ untranslated region (UTR) of the target RNA.

Embodiment 74. The engineered polynucleotide of any one of embodiments 1-72, wherein the targeting sequence is configured to at least partially associate with at least a portion of a translation initiation site.

Embodiment 75. The engineered polynucleotide of any one of embodiments 1-72, wherein the targeting sequence is configured to at least partially associate with at least a portion of an intronic region of the target RNA.

Embodiment 76. The engineered polynucleotide of any one of embodiments 1-72, wherein the targeting sequence is configured to at least partially associate with at least a portion of an exonic region of the target RNA.

Embodiment 77. The engineered polynucleotide of any one of embodiments 1-76, wherein the engineered polynucleotide is about 80 nucleotides to about 600 nucleotides.

Embodiment 78. The engineered polynucleotide of any one of embodiments 1-77, wherein the engineered polynucleotide comprises a sequence that has at least 80% identity to any one of SEQ ID NO: 1-SEQ ID NO: 33 or a variant thereof.

Embodiment 79. The engineered polynucleotide of embodiment 78, wherein the snRNA sequence comprises a sequence that has at least 90% identity to any one of SEQ ID NO: 1-SEQ ID NO: 33 or a variant thereof. d

Embodiment 80. The engineered polynucleotide of any one of embodiments 1-79, wherein the engineered polynucleotide comprises a first spacer domain 5′ to the targeting sequence.

Embodiment 81. The engineered polynucleotide of embodiment 80, wherein the engineered polynucleotide comprises a second spacer domain distinct from or identical to the first spacer domain.

Embodiment 82. The engineered polynucleotide of embodiment 80 or 81, wherein the first spacer domain, the second spacer domain or both comprises a polynucleotide sequence of: 5′ AUAUA 3′.

Embodiment 83. The engineered polynucleotide of embodiment 80 or 81, wherein the first spacer domain, the second spacer domain or both comprises a polynucleotide sequence of: 5′ AUAAU 3′.

Embodiment 84. The engineered polynucleotide of any one of embodiments 70-83, wherein the engineered polynucleotide comprises a first ribozyme domain on a 5′ end and a second ribozyme domain on a 3′ end.

Embodiment 85. The engineered polynucleotide of embodiment 84, wherein the first or second ribozyme is independently selected from the group consisting of a Hammerhead ribozyme, a glmS ribozyme, an HDV-like ribozyme, an R2 element, a peptidyl transferase 23S rRNA, a GIR1 branching ribozyme, a leadzyme, a group II intron, a hairpin ribozyme, a VS ribozyme, a CPEB3 ribozyme, a CoTC ribozyme, and a group I intron.

Embodiment 86. A vector comprising or encoding the engineered polynucleotide of any one of embodiments 1-85.

Embodiment 87. The vector of embodiment 86, wherein the vector comprises a liposome, a nanoparticle, or a dendrimer.

Embodiment 88. The vector of embodiment 86, wherein the vector is a viral vector.

Embodiment 89. The vector of embodiment 88, wherein the viral vector is an adeno-associated viral (AAV) vector.

Embodiment 90. The vector of embodiment 89, wherein the AAV vector is an AAV2 vector, AAV5 vector, AAV8 vector, AAV9 vector, or a hybrid of any of these.

Embodiment 91. The vector of embodiment 89 or 90, wherein the viral vector is a self-complementary adeno-associated viral (scAAV) vector

Embodiment 92. The vector of any one of embodiments 89-90, wherein the viral vector is a single-stranded AAV vector.

Embodiment 93. An isolated cell comprising the engineered polynucleotide of any of embodiments 1-85, the polynucleotide encoding the engineered polynucleotide of any of embodiments 1-85, or the vector of any one of embodiments 86-92.

Embodiment 94. The isolated cell of embodiment 93, wherein the isolated cell is a T cell.

Embodiment 95. A pharmaceutical composition in unit dose form comprising the engineered polynucleotide of any of embodiments 1-85, a polynucleotide encoding engineered polynucleotide of any of embodiments 1-85, or the vector of any one of embodiments 86-92; and a pharmaceutically acceptable: excipient, diluent, or carrier.

Embodiment 96. A method of treating or preventing a condition in a subject in need thereof, comprising administering to the subject an effective amount of the engineered polynucleotide of any one of embodiments 1-85, a polynucleotide encoding engineered polynucleotide of any of embodiments 1-85, the vector of any one of embodiments 86-92, or the pharmaceutical composition of embodiment 95.

Embodiment 97. The method of embodiment 96, wherein the condition is Duchenne's Muscular Dystrophy (DMD), Rett's syndrome, Charcot-Marie-Tooth disease, Alzheimer's disease, a taupathy, Parkinson's disease, alpha-1 anti trypsin deficiency, or Stargardt's disease.

Embodiment 98. The method of embodiment 96, wherein the condition is associated with a mutation in a gene selected from the group consisting of RAB7A, ABCA4, SERPINA1, SERPINA1 E342K, HEXA, LRRK2, SNCA, DMD, APP, Tau, CFTR, ALAS1, ATP7B, ATP7B G1226R, HFE C282Y, LIPA c.894 G>A, PCSK9 start site, or SCNN1A start site, a fragment any of these, and any combination thereof.

Embodiment 99. The method of any one of embodiments 96-98, wherein the administering is inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration. Delivery can include topical administration (such as a lotion, a cream, an ointment) to an external surface of a surface, such as a skin. In some cases, administration is by parenchymal injection, intra-thecal injection, intra-ventricular injection, intra-cisternal injection, intravenous injection, or intranasal administration or any combination thereof.

Embodiment 100. The method of any one of embodiment 96-99, further comprising administering an additional treatment.

Embodiment 101. The method of embodiment 100, wherein the additional treatment is administered concurrently or consecutively.

Embodiment 102. The method of any one of embodiments 96-101, wherein the administering is performed at least once a week.

Embodiment 103. The method of any one of embodiments 96-102, wherein the subject has been diagnosed with the disease or condition by an in vitro diagnostic prior to the administering.

Embodiment 104. The method of any one of embodiments 96-103, wherein the subject is human.

Embodiment 105. A kit comprising the engineered polynucleotide of any one of embodiments 1-85 in a container, a polynucleotide encoding engineered polynucleotide of any of embodiments 1-85 in a container, the vector of any one of embodiments 86-92 in a container, or the pharmaceutical composition of embodiment 95 in a container.

Embodiment 106. A method of making a pharmaceutical composition, comprising contacting a pharmaceutically acceptable: excipient, carrier, or diluent with at least one of the engineered polynucleotide of any of embodiments 1-85, a polynucleotide encoding engineered polynucleotide of any of embodiments 1-85, or the vector of any one of embodiments 86-92.

Embodiment 107. A method of making a kit, comprising placing at least in part, into a container:
(a) the engineered polynucleotide of any one of embodiments 1-85;
(b) a polynucleotide encoding engineered polynucleotide of any of embodiments 1-85;
(c) the vector of any one of embodiments 86-92; or
(d) the pharmaceutical composition of embodiment 95.

### EXAMPLES

The following examples are intended to illustrate but not limit the disclosure. While they are typical of those that might be used, other procedures known to those skilled in the art may alternatively be used.

### Example 1: Assessing snRNA Promoter and 3′ Hairpin Effects on Specific Guide RNA Editing

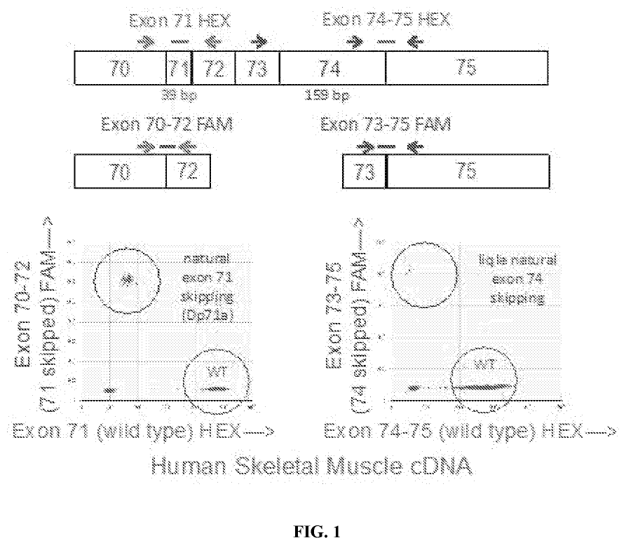

In this example, additional features added to the guide RNA were tested for their ability in effecting a guide RNAs' ability to edit or perform exon skip. FIG. 1 shows an example exon skipping ddPCR assay used to assess the presence of exon skipping activity.

### Addition of the U7 and UI Hairpins to the 3′ End of the Guide RNA

In this study, guide RNAs that were expressed under different snRNA promoters were tested on their specific guide RNA editing.

To test whether the snRNA promoter-driven guide RNA expression would affect editing, 100 nucleotide guide RNAs targeting the human RAB7A 3′UTR, the splice acceptor of human DMD exon 71, or the splice acceptor of human DMD exon 74 were expressed using hU6, hU7, or hU1 promoters with or without a corresponding 3′ hairpin (SmOPT mU7, SmOPT hU7, or hU1 hairpin). The termination sequences used match those of the promoter. 293T cells were transfected with 1 μg of plasmid expressing the guide RNA construct with or without human ADAR2 overexpression.

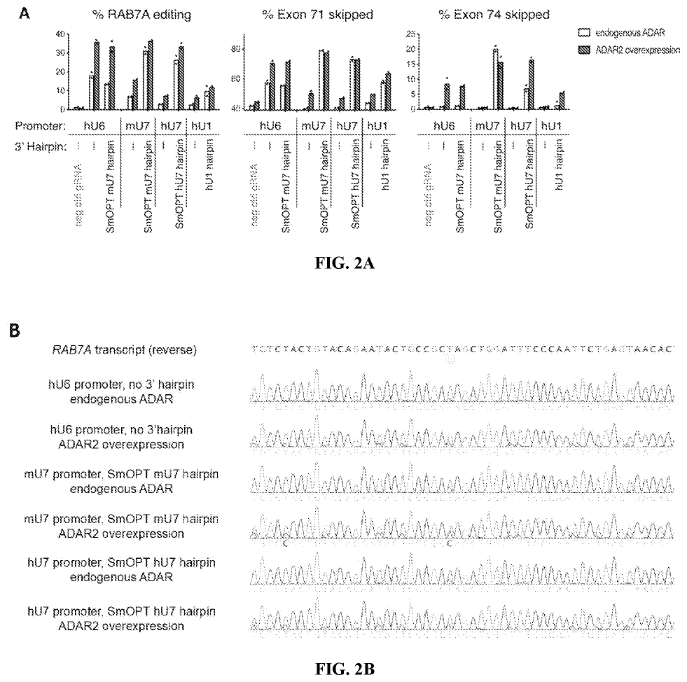

In FIG. 2A, The U7 or U1 promoters, combined with a 3′ hairpin, enhanced RAB7A ADAR editing and DMD exon 71 or exon 74 skipping (as measured by ddPCR). These constructs exhibited similar editing even in the absence of ADAR2 overexpression. FIG. 2B shows Sanger sequencing chromatograms to demonstrate the specific at editing of the adenosine in the RAB7A 3′UTR as noted by the box. A reverse primer is used for Sanger sequencing; thus, an A-to-G edit appears as a T-to-C mutation.

### Example 2: Editing of RAB7A and SNCA with U7 Promoter Containing Guide RNAs

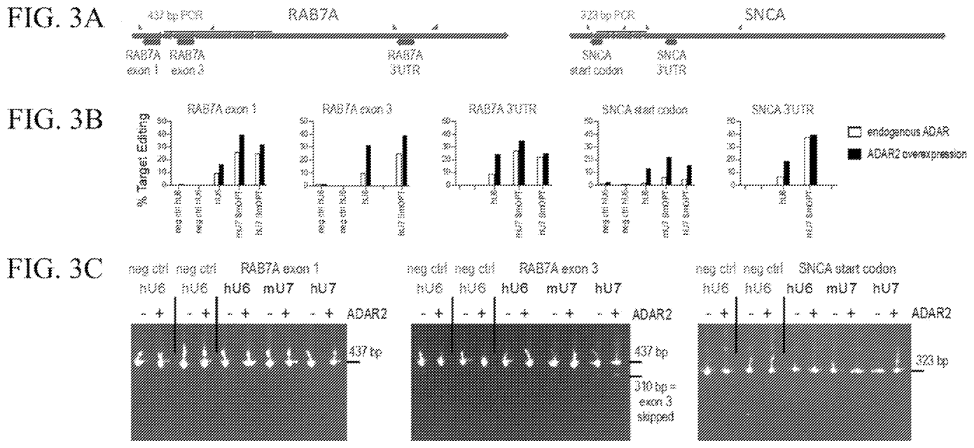

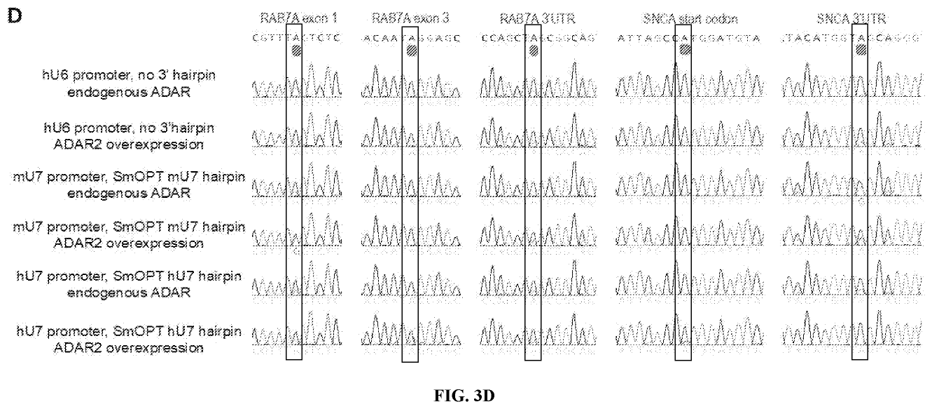

In this example, different regions along RAB7A and alpha-synuclein (SNCA) were edited using different guide RNA constructs. For RAB7A, exons 1, 3, and the 3′ UTR were targeted for editing, whereas for SNCA, the start codon and the 3′ UTR were targeted. FIG. 3A shows a schematic of the exon structure of human RAB7A and SNCA. Exons are shown as gray segments; the coding region is denoted as a black line above. Locations of the guide RNA targeting sites are shown; PCR primers are also shown.

100 nt guide RNAs targeting human RAB7A exon 1, exon 3, or 3′UTR, or human SNCA start codon or 3′UTR were expressed using the hU6 promoter without a 3′ hairpin or the mU7 or hU7 promoters with a 3′ SmOPT U7 hairpin. FIG. 3B summarizes the results of the editing using the different guide RNA constructs in the presence or absence of ADAR2 overexpression. U7 promoters combined with a 3′ SmOPT U7 hairpin enhanced ADAR editing at each target site (measured by Sanger sequencing). While constructs targeting the 3′UTRs worked equally well under endogenous versus overexpressed ADAR levels, constructs targeting other areas still benefited from ADAR2 overexpression.

To confirm whether differential exon skipping occurred, cDNA derived from the edited transcripts were isolated and PCR amplified using the denoted primers. The RAB7A primers, which span part of the coding determining sequence of RAB7A, generate a 437 bp amplicon if the exon structure is maintained. If exon 3 of RAB7A is skipped, a 310 bp amplicon is expected. Using the SNCA primers, a 323 bp PCR amplicon is expected. FIG. 3C shows minimal RAB7A exon 3 skipping and no detectable exon skipping from guide RNAs targeting the RAB7A exon 1 or SNCA start codon.

FIG. 3D shows Sanger sequencing chromatograms showing specific editing at the target adenosine of the indicated transcripts. The box indicates the on-target editing site.

### Example 3: On and Off Target Editing of the Human SNCA Start Codon and 3′ UTR in HEK 293 and K562 Cells

In this example, different regions of SNCA were edited using different cell lines and transfection methods. 100 nt guide RNAs targeting the human SNCA start codon or human SNCA 3′UTR were expressed using a hU6 promoter with no hairpin, a mU7 promoter with a 3′ SmOPT mU7 hairpin, a hU7 promoter with a 3′ SmOPT hU7 hairpin, or a hU1 promoter with a 3′ SmOPT mU7 hairpin. Disclosed herein are compositions of engineered polynucleotides under a U7 or U1 promoter and also comprising a SmOPT U7 hairpin sequence. Said engineered polynucleotides can hybridize to a target RNA sequence corresponding to SNCA, to facilitate ADAR-mediated editing of an adenosine.

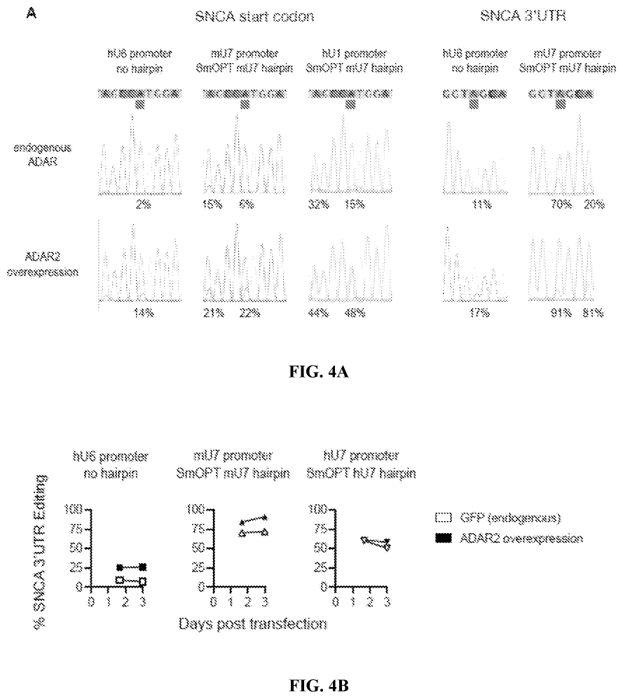

FIG. 4A shows the effect of profiling different sites of SNCA and measuring ADAR-mediated editing efficiency. HEK293 cells were transfected with plasmid containing the guide sequences and RNA editing was measured by Sanger sequencing two days later. In some cases, ADAR2 was overexpressed. Assessment of the A/C mismatch site (red box) was evaluated to determine the editing efficiency in each condition. In some instances, editing efficiency below 5% may be below background levels.

FIG. 4B Editing of the SNCA 3′UTR was assessed by transfection of the engineered polynucleotides in K562 cells which overexpress SNCA. 1.5 μg of guide RNA-expressing plasmid was transfected into 2×10⁵ SNCA-overexpressing K562 cells via nucleofection (Lonza). RNA editing was measured 40 and 72 hours after transfection. Open symbols indicate experiments where cells were transfected with a GFP expressing vector; solid symbols indicate transfection with an ADAR2 overexpression vector.

### Example 4: RNA Editing Using Linear and Circular Guide RNAs

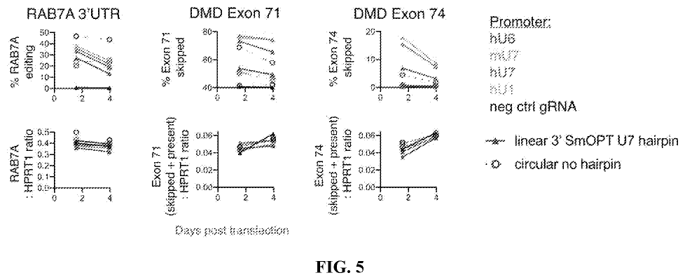

Disclosed herein are compositions of engineered polynucleotides under control of different mammalian snRNA promoters. These constructs are linear engineered polynucleotide constructs with a 3′ SmOPT U7 hairpin or a circular guide RNA construct lacking a hairpin. FIG. 5 demonstrates that a human U1 promoter can also be paired with a 3′ SmOPT sequence and U7 hairpin for guide RNA editing with minimal knockdown of transcript levels.

100 nt guide RNAs targeting the human RAB7A 3′UTR, human DMD exon 71 Splice Acceptor, or human DMD exon 74 Splice Acceptor were expressed using the hU6, mU7, hU7, or hU1 promoters with a 3′ SmOPT U7 hairpin and U7 termination sequence. Alternatively, these 100 nt guide RNAs were expressed using the hU6, mU7, hU7, or hU1 promoters with circularizing RtcB ribozyme sites (no SmOPT or U7 hairpin). 293T cells were transfected with 1 ug of plasmid expressing the guide RNA construct (endogenous ADAR levels); RNA was measured at the indicated time point post transfection by ddPCR.

U7 or U1 promoters expressing guide RNAs with a 3′ SmOPT U7 hairpin resulted in high levels of RAB7A editing and DMD exon skipping; however, the U6 promoter, when expressing these same guide RNA constructs, was notably weaker. In contrast, the U6 promoter expressing the circular guide RNAs (no hairpin) resulted in the highest levels of RAB7A editing, but a more moderate level of DMD exon skipping.

### Example 5: Addition of the hnRNP A1 Binding Domain to the 5′ End of the Engineered Polynucleotides

Adding hnRNP A1 binding domains [UAGGGW], where W is A/U, at the 5′ end of the guide RNA opposite a 3′ SmOPT U7 hairpin further increases RAB7A editing and DMD exon 74 skipping.

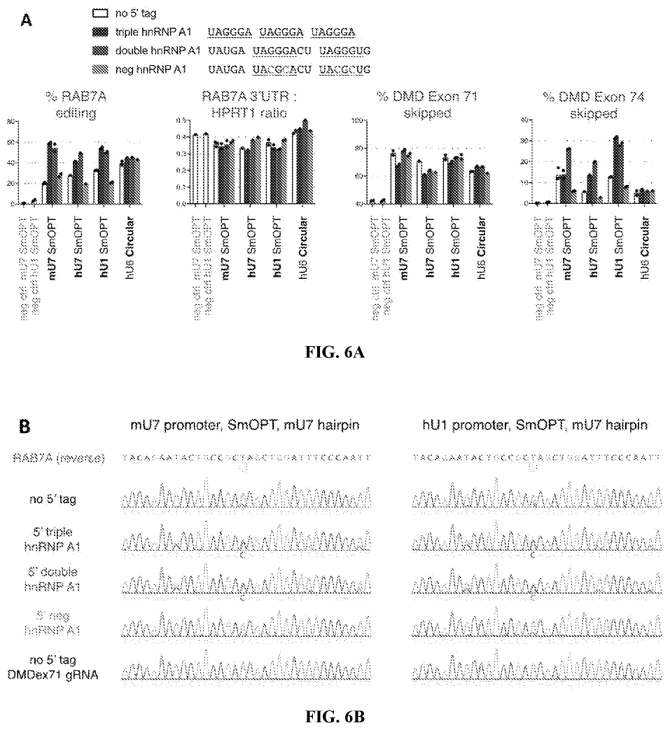

100 nt guide RNAs targeting the human RAB7A 3′UTR, human DMD exon 71 Splice Acceptor, or human DMD exon 74 Splice Acceptor were expressed using the mU7 or hU1 promoters with a 3′ SmOPT mU7 hairpin and mU7 termination sequence. Alternatively, these 100 nt guide RNAs were expressed using the hU6 promoter with circularizing RtcB ribozyme sites from Litke and Jaffrey 2019 (no SmOPT or U7 hairpin). Added at the 5′ end of the guide RNA was either: no tag; a triple hnRNP A1 binding domain; a double hnRNP A1 binding domain; or a mutated domain that does not bind hnRNP A1 as a negative control. 293T cells were transfected with 1 ug of plasmid expressing the guide RNA construct (endogenous ADAR levels); RNA was measured 42 hr later. FIG. 6A shows the hnRNP binding domains, when combined with the U7/U1 promoters and a 3′ SmOPT U7 hairpin, increased RAB7A ADAR editing and DMD exon 74 skipping (measured by ddPCR). Unfortunately, the hnRNP domains did not improve the U6 circular guide RNAs. RAB7A editing does not alter the transcript expression level relative to the HPRT1 housekeeping gene. DMD exon 71 skipping was also not improved, possibly because these conditions have already maximized the amount of exon 71 skipping (given transfection efficiency and transcript turnover). FIG. 6B shows Sanger sequencing chromatograms showing specific editing at the target adenosine in the RAB7A 3′UTR (box). Since a reverse primer was used for sequencing, an A>G edit appears as T>C.

### Example 6: RNA Editing with Guides Having an SmOPT Sequence and a U7 Stem-Loop Hairpin

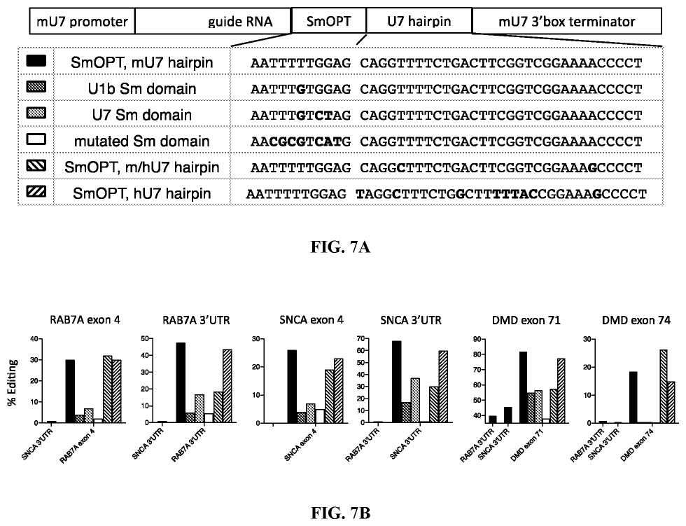

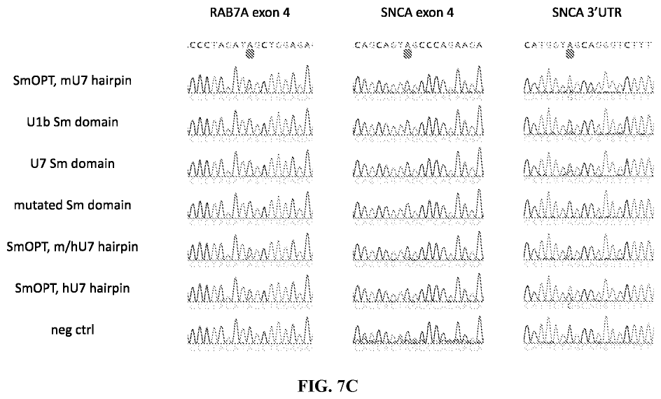

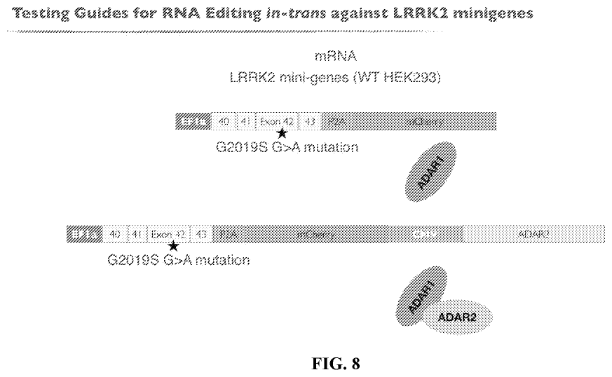

To dissect which features of a 3′ SmOPT U7 hairpin are required for enhanced editing, variations of the SmOPT sequence and variations of the U7 stem-loop hairpin were compared. 100 nt guide RNAs targeting the human RAB7A exon 4, RAB7A 3′UTR, SNCA exon 4, SNCA 3′UTR, DMD exon 71 Splice Acceptor, or DMD exon 74 Splice Acceptor were expressed using the mU7 promoter and mU7 termination sequence along with a variation on the 3′ SmOPT mU7 stem-loop hairpin sequence. 293T cells were transfected with 1 ug of plasmid expressing the guide RNA construct (endogenous ADAR levels); RNA was measured 42 hr later. FIG. 7A lists the sequence variations tested. FIG. 7B shows that an SmOPT sequence, but not any of the natural or mutated variations, is necessary for full editing of the target transcripts. FIG. 7B also shows that the mouse or human U7 stem-loop hairpin, or a hybrid mouse/human U7 stem-loop hairpin, can suffice for editing. FIG. 7C shows example Sanger sequencing chromatograms showing specific editing at the target adenosine of the indicated transcripts. The box indicates the on-target editing site.

### Example 7: Guide RNAs Against LRRK2 with SmOPT and a U7 Hairpin

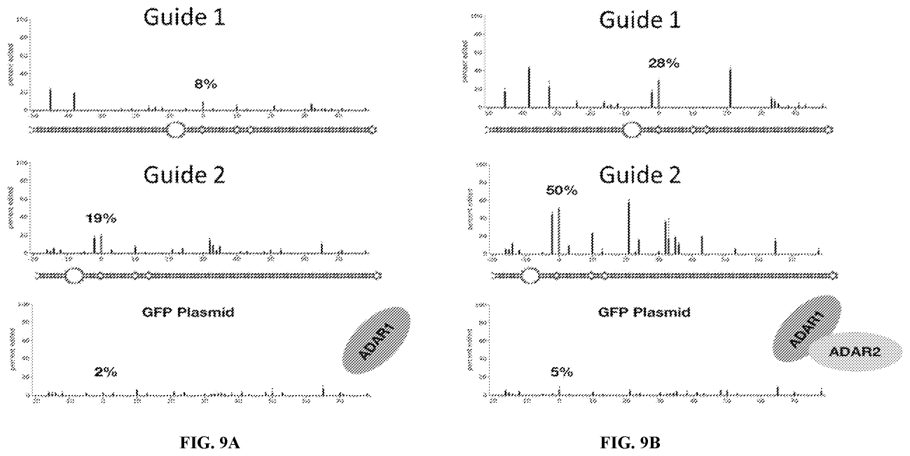

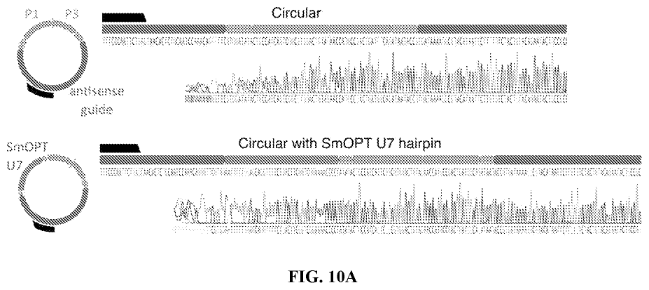

This example describes constructs of the present disclosure encoding for a guide RNA under the control of a U1 promoter, an SmOPT sequence, and a U7 hairpin, wherein the guide RNA is designed to target LRRK2, a gene implicated in Parkinson's disease. Two guide RNA designs targeting the LRRK2 G2019S mutation were tested, both with SmOPT and a U7 hairpin. The first guide was SEQ ID NO: 18. The second guide was SEQ ID NO: 19. Guide RNAs were tested for their ability to facilitate ADAR-mediated RNA editing of the G2019S LRRK2 mutation in WT HEK293 cells transfected with a piggyBac vector carrying a LRRK2 minigene. WT HEK293 cells naturally express ADAR1. In experiments in which RNA editing mediated via ADAR1 and ADAR2 was tested, ADAR2 was overexpressed in cells via the same piggyBac vector carrying the LRRK2 minigene. Schematics of the piggyBac constructs are shown in FIG. 8. Experiments were conducted in the presence of ADAR1 only (FIG. 9A) or ADAR1 and ADAR2 (FIG. 9B). A GFP plasmid was used as a control. FIG. 9A and FIG. 9B show that guide RNAs containing SmOPT and a U7 hairpin facilitated an on-target editing efficiency of 8% in the presence of ADAR1 only and 28% in the presence of ADAR1 and ADAR2. FIG. 9A and FIG. 9B show that guide RNAs containing SmOPT and a U7 hairpin facilitated an on-target editing efficiency of 19% in the presence of ADAR1 only and 58% in the presence of ADAR1 and ADAR2. Further experimentation demonstrated that the first guide (SEQ ID NO: 18) had a Gibbs free energy (delta G) of −161.98 kcal/mol and the second guide (SEQ ID NO: 19) had a delta G of −169.44 kcal/mol. The structures of both guide RNAs are shown beneath the graphs in FIG. 9A-FIG. 9B. As seen in the structural diagrams, the second guide (SEQ ID NO: 18) formed a longer continuous stretch of duplex RNA with the target RNA.

### Example 8: Guide RNA Containing a 3′ SmOPT Sequence and U7 Hairpin can be Circularized and Expressed by U7 or U6 Promoters to Produce ADAR Editing

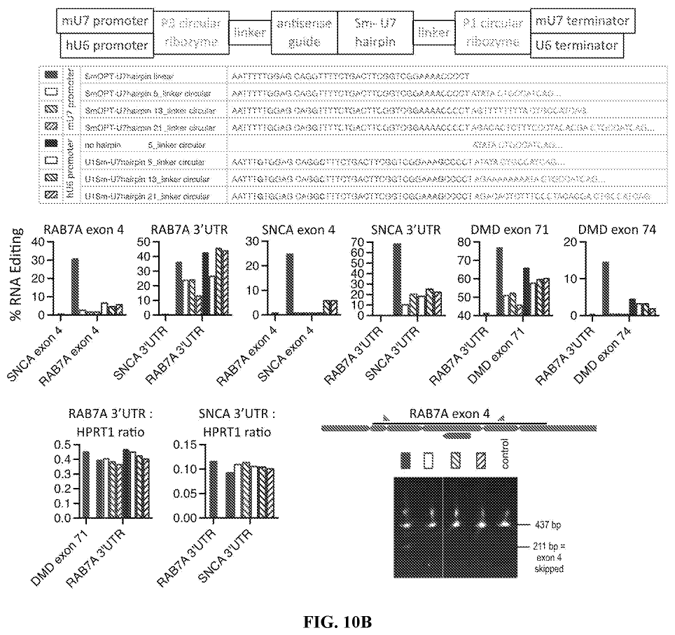

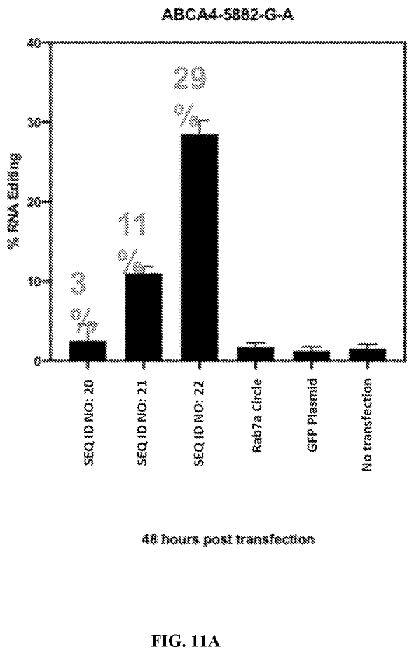

100 nt antisense (targeting) guide RNAs with a 3′ SmOPT sequence and U7 hairpin were inserted between P3 and P1 RtcB circular ribozyme sites and expressed using the mU7 or hU6 promoters. 293T cells were transfected with 1 μg of plasmid expressing the guide RNA construct (endogenous ADAR levels); RNA was measured 41 hr later. FIG. 10A illustrates the circular RNA forms; Sanger sequencing with a guide-specific primer (black) shows that the ribozyme sites have been precisely ligated together, with the antisense guide and 3′ SmOPT U7 hairpin inside the circular RNA.

FIG. 10B lists several sequence variations of the Sm-binding domain, U7 stem-loop hairpin, and RNA linker sequences that were cloned between P3 and P1 RtcB circular ribozyme sites (top panel). Included within the circular RNA, before the Sm-binding domain, were 100 nt antisense guide RNAs targeting human RAB7A exon 4, RAB7A 3′UTR, SNCA exon 4, SNCA 3′UTR, DMD exon 71 Splice Acceptor, or DMD exon 74 Splice Acceptor. Guide RNAs were expressed using either the mU7 or hU6 promoter, each with its corresponding terminator sequence. In addition to a linear SmOPT U7 hairpin guide RNA, circularized guide RNAs containing an Sm-binding domain and U7 stem-loop hairpin could also edit the target transcript, as measured by Sanger sequencing (middle panel). As a negative control, linear SmOPT U7 hairpin guide RNAs that targeted a different gene were used. Furthermore, neither the linear or circular forms of the SmOPT U7 hairpin guide RNAs altered the gene expression level of the RAB7A 3′UTR or SNCA 3′UTR target transcripts, as compared to the HPRT1 housekeeping gene and measured by ddPCR (bottom left panel). The linear SmOPT U7 hairpin guide RNA caused only minimal inadvertent skipping of RAB7A exon 4, and the circular SmOPT U7 hairpin guide RNAs showed no detectable exon skipping, as measured by PCR and gel electrophoresis (bottom right panel).

### Example 9: Guide RNAs Against ABCA4 with SmOPT and a U7 Hairpin

This example describes constructs of the present disclosure encoding for guide RNAs under the control of a promoter, an SmOPT sequence, and a U7 hairpin, wherein the guide RNA is designed to target an ABCA4 missense mutation, implicated in Stargardt's disease.

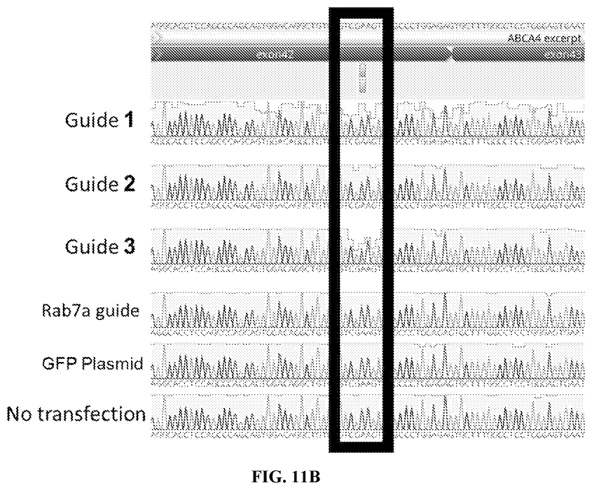

An initial set of experiments demonstrated the improvement in RNA editing of ABCA4 observed in guides incorporating the SmOPT sequence and a U7 hairpin. HEK293 cells naturally expressing ADAR1 were transfected with a piggyBac vector carrying an ABCA4 minigene having the 5882 G->A mutation and ADAR2. Various guides were tested including two U6 driven guide RNAs (SEQ ID NO: 20 and SEQ ID NO: 21) and a U1 driven guide RNA containing the SmOPT sequence and U7 hairpin (SEQ ID NO: 22). Negative controls included a circular guide RNA to a different gene (Rab7A), GFP plasmid alone, and no transfection. As shown in FIG. 11A and FIG. 11B, the inclusion of the U7 SmOPT sequence and a U7 hairpin increased RNA editing.

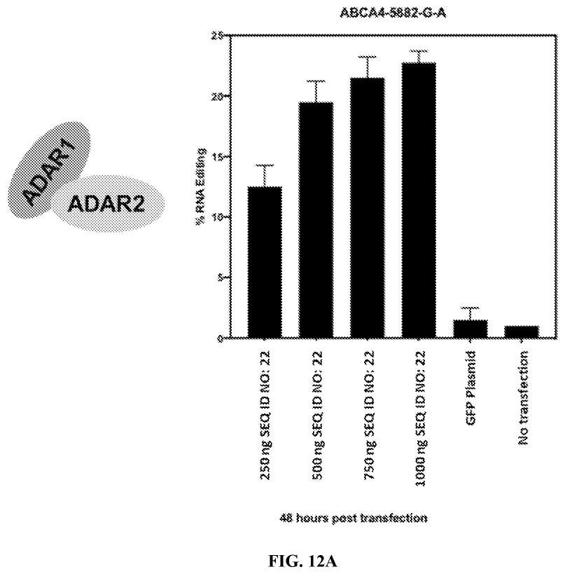

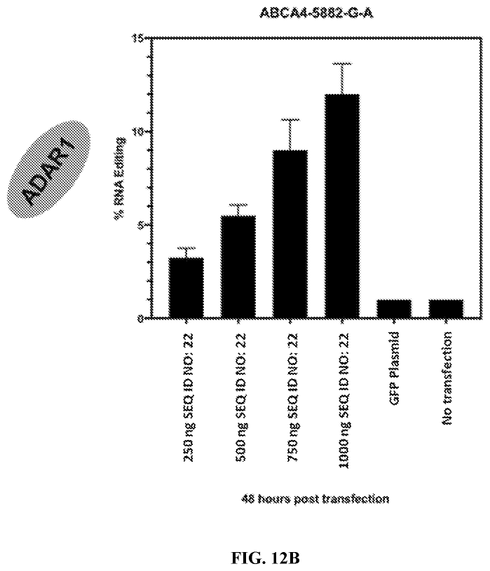

Subsequent experiments evaluated guide RNAs to target the ABCA4 5882 G->A mutation, where engineered polynucleotide encoding for the guide RNA also encoded for an SmOPT sequence and a U7 hairpin. FIG. 12A shows percent RNA editing in cells by ADAR1 and ADAR2 for multiple doses of constructs encoding for a guide RNA targeting a mutation in ABCA4, an SmOPT sequence, and a U7 hairpin, where expression is driven by a U1 promoter. As described above, HEK293 cells naturally express ADAR1. For FIG. 12A, HEK293 cells were transfected with a piggyBac vector carrying an ABCA4 minigene having the 5882 G->A mutation and ADAR2. FIG. 12B shows percent RNA editing in cells by ADAR1 for multiple doses of constructs encoding for a guide RNA targeting a mutation in ABCA4, an SmOPT sequence, and a U7 hairpin, where expression is driven by a U1 promoter. HEK293 cells were transfected with a piggyBac vector carrying just the ABCA4 minigene having the 5882 G->A mutation. In both FIG. 12A and FIG. 12B, plasmids encoding for the guide RNA, SmOPT sequence, and U7 hairpin were dosed at 250 ng, 500 ng, 750 ng, or 1000 ng. A GFP plasmid and no transfection served as negative controls. Results showed a dose dependency in percent RNA editing of the ABCA4 5882 G->A mutation. Sequences targeting ABCA4 include SEQ ID NO: 20, SEQ ID NO: 21, and SEQ ID NO: 22.

### Example 10: Guide RNAs Against SNCA with a SmOPT Sequence and a U7 Hairpin

This example describes constructs of the present disclosure encoding for guide RNAs under the control of a U1 promoter, an SmOPT sequence, and a U7 hairpin, where the guide RNA is designed to target a start site, or translation initiation site (TIS) (also referred to as translation start site (TSS)) in the SNCA gene. Guide constructs were designed to target SNCA TIS adenosine at nucleotide position 26 of Exon 2 (corresponding to nucleotide position 264 of most SNCA variants, including Exon 1 and Exon 2). HEK293 cells were transfected with a plasmid encoding for a guide RNA of interest and RNA editing was assessed 48 hours post-transfection. RNA editing was assessed for ADAR1 only, which is naturally expressed by HEK293 cells, and ADAR1 and ADAR2. In the latter experiment, HEK293 cells were co-transfected with a piggybac vector encoding for ADAR2. Levels of RNA editing were quantified by Sanger sequencing and analyzed using a sequencing analysis script (EditADAR).

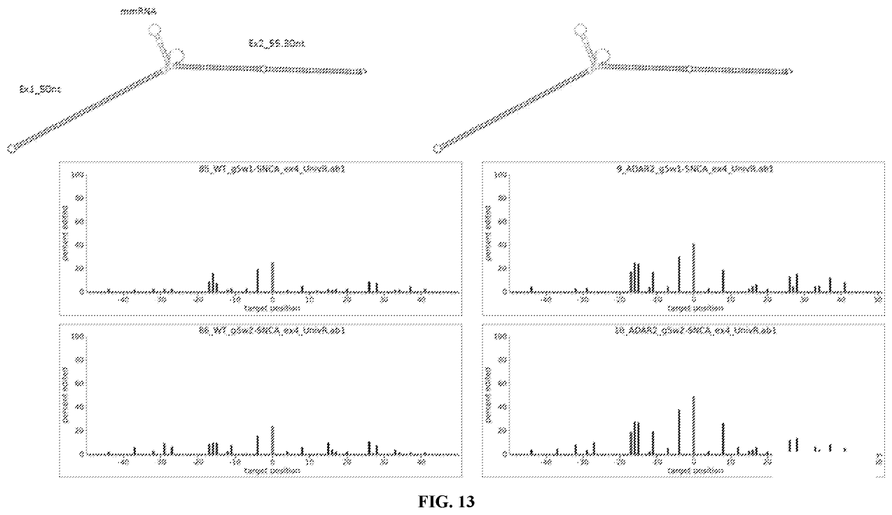

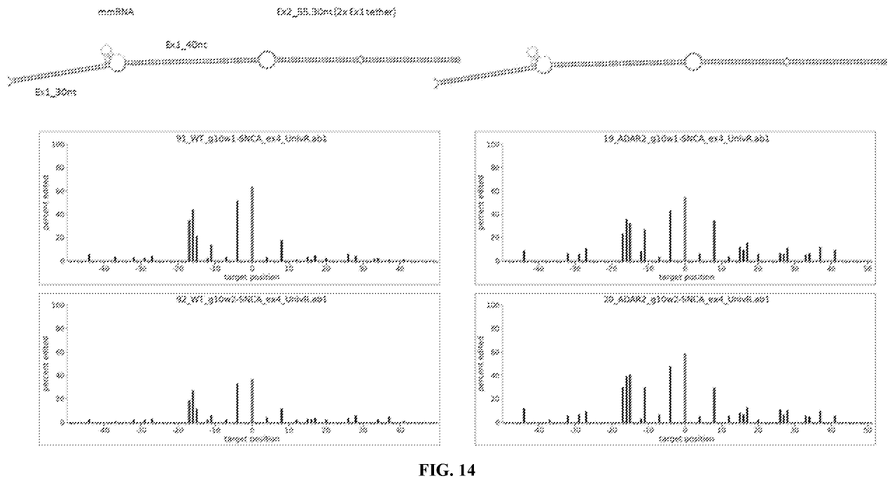

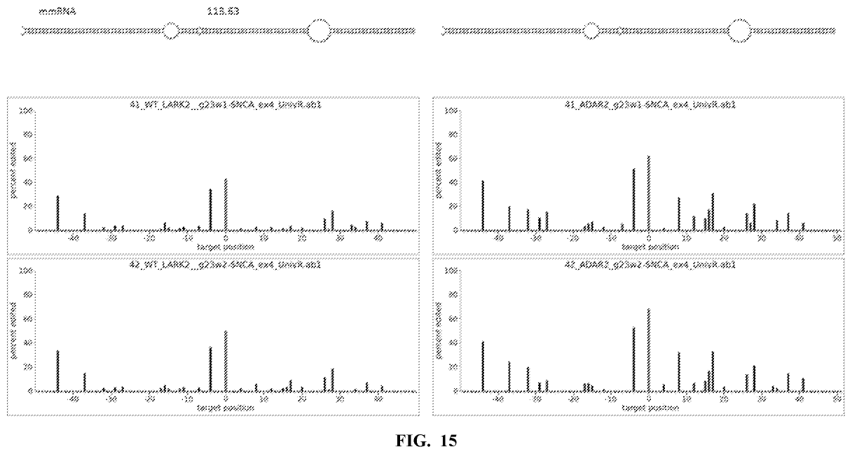

FIG. 13-FIG. 15 show plots of RNA editing at the target A to be edited (“0” on the x-axis) and at RNA editing at off-target positions (represented as black bars at positions that are not “0”). Biological replicates are shown in each column. High levels of RNA editing of SNCA were observed in several guide RNA constructs. Negative controls run included guide RNAs not targeting the target SNCA sequence. While mRNA knockdown by qPCR wasn't observed, it is still likely that protein knockdown was achieved. Guide constructs shown in FIG. 13, FIG. 14, and FIG. 15 exhibited high levels of RNA editing and a high ratio of on-target to off-target edits. The Guide shown in FIG. 14 comprises an oligo tether, which is a segment of the guide adjacent to the targeting sequence that has non-continuous complementarity to the target strand.

Sequences of guides shown in FIG. 13-FIG. 15 are described below in TABLE 2. TABLE 2 below also describes features formed upon hybridization of a given guide to a target RNA, the percent on-target RNA editing observed, on-target editing as a percentage of total RNA editing that is on-target RNA editing, and on-target editing as a percentage of RNA editing at the target in the start site and downstream of the start site in the coding region.

TABLE 2
Guide RNA Sequences

| | | Percent |
|---|---|---|
| | | On-Target |
| | | Editing |
| | | [ADAR1, |
| SEQ ID | | ADAR1 + |
| NO | Sequence | ADAR2] |
| Target | | — |
| Sequence | | |
| SEQ ID | TAATCTTTGAAA | [24, 45] |
| NO: 31 | GTCCTTTCATGA | |
| | ATACATCCACG | |
| | GCTAATGAATTC | |
| | CTTTACACCACA | |
| | TTAGCCAGAAG | |
| | GCTTGAAGGCA | |
| | AGGCGTGAGGG | |
| | AGCGCCCAGGA | |
| | CGCTCTCGGAG | |
| | ATATATAAATTT | |
| | TTGGAGCAGGT | |
| | TTTCTGACTTCG | |
| | GTCGGAAAACC | |
| | CCT | |
| SEQ ID | TAATCTTTGAAA | [50, 57] |
| NO: 32 | GTCCTTTCATGA | |
| | ATACATCCACG | |
| | GCTAATGAATTC | |
| | CTTTACACCACA | |
| | AGGGGCGAATG | |
| | GCCACTCCCAG | |
| | TTCTCCGCTCAC | |
| | GAGGGTGGAAA | |
| | TAATTAAGGCG | |
| | TGAGGGAGCGC | |
| | CCAGGACGCTC | |
| | TCATATATAAAT | |
| | TTTTGGAGCAG | |
| | GTTTTCTGACTT | |
| | CGGTCGGAAAA | |
| | CCCCT | |
| SEQ ID | TAATTTTCTCAG | [49, 68] |
| NO: 33 | CAGCAGCCACA | |
| | ACTCCGAGGAA | |
| | CCCCTTTGAAA | |
| | GTCCTTTCATGA | |
| | ATACATCCACG | |
| | GCTAAACTTCTC | |
| | CTTTACACCACA | |
| | CTGTCGTCGAA | |
| | TGGCCACTCCC | |
| | AGTATATATAAA | |
| | TTTTTGGAGCA | |
| | GGTTTTCTGACT | |
| | TCGGTCGGAAA | |
| | ACCCCT | |

### Example 11: Guide RNAs Against the Human SOD1 Start Codon with SmOPT and a U7 Hairpin from Plasmid Transient Transfection or a Single-Copy Integration of Construct

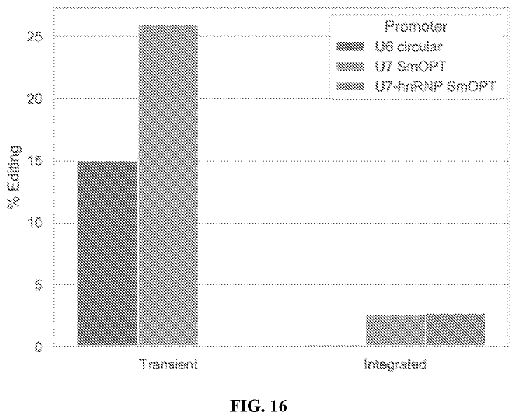

100 nt guide RNAs targeting the human SOD1 start codon were expressed using a hU6 promoter with circularizing RtcB ribozyme sites (no SmOPT or U7 hairpin); a mU7 promoter with a 3′ SmOPT mU7 hairpin; or a mU7 promoter with a 5′ double hnRNP A1 binding domain and 3′ SmOPT hU7 hairpin. Said engineered polynucleotides can hybridize to a target RNA sequence corresponding to SOD1, to facilitate ADAR-mediated editing of an adenosine. FIG. 16 shows editing of the human SOD1 start codon where the engineered polynucleotide was delivered to HEK 293T cells either by plasmid transient transfection, or integrated into the genome as a single copy. For transient delivery, 1 ug of plasmid was transfected into HEK 293T cells (endogenous ADAR) and the percent of editing was measured 2 days later. For integrated delivery, a single copy of the guide RNA expression construct was inserted into the genome and the percent of editing was measured post antibiotic selection over a week later.

### Example 12: Guide RNAs Against SERPINA1 with SmOPT and a U7 Hairpin

This example describes constructs of the present disclosure encoding for guide RNAs under the control of a promoter, an SmOPT sequence, and a U7 hairpin, wherein the guide RNA is designed to target a SERPINA1 missense mutation (SERPINA1, G to A mutation at position 9989 yielding the SERPINA1 E342K mutation), implicated in Alpha-1 antitrypsin deficiency.

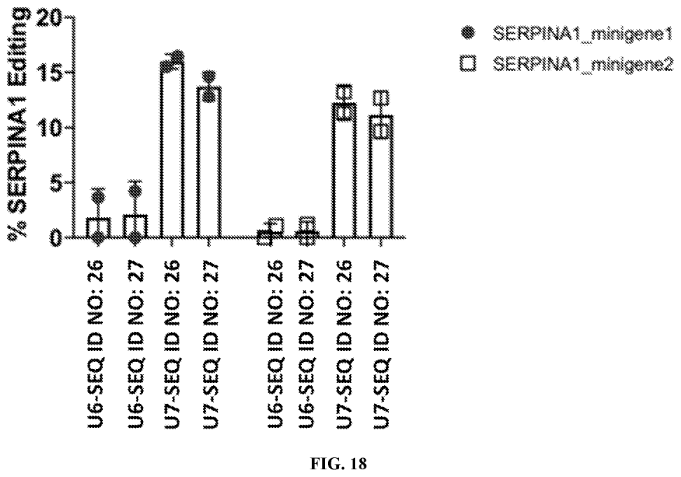

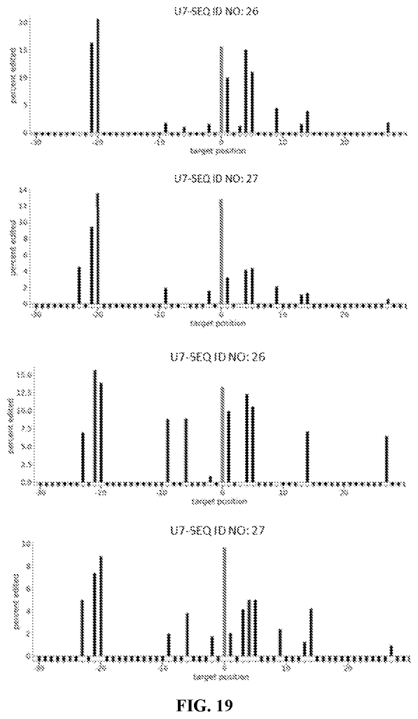

Briefly, SERPINA1 minigenes were transfected into K562 cells expressing endogenous ADAR1 via a piggyBac vector and cells were selected via puromycin selection. SERPINA1 minigenes integrated into K562 cells included a SERPINA1 minigene1 having a full length 3′ UTR or a SERPINA1 minigene2 having a truncated 3′ UTR. Both minigenes carried the G to A 9989 mutation of interest. K562 cells (2×10\hat{ }5 cells) were electroporated with plasmids encoding for a guide RNA operably linked to the U7 hairpin and an SmOPT sequence. Expression was driven under a mouse U7 promoter. 24 hours post-electroporation, RNA was isolated, cDNA was synthesized via RT-PCR and RT-PCR products were sequenced via Sanger sequencing to quantify percent RNA editing. Control guide RNAs lacked the U7 hairpin and the SmOPT sequence and expression was driven under a U6 promoter. FIG. 18 and FIG. 19 show editing of SERPINA1 using the guide RNA sequences. Guide RNA sequences utilized include SEQ ID NO: 26 and SEQ ID NO: 27.

### Example 13: Guide RNAs Against RAB7A with SmOPT and a U7 Hairpin

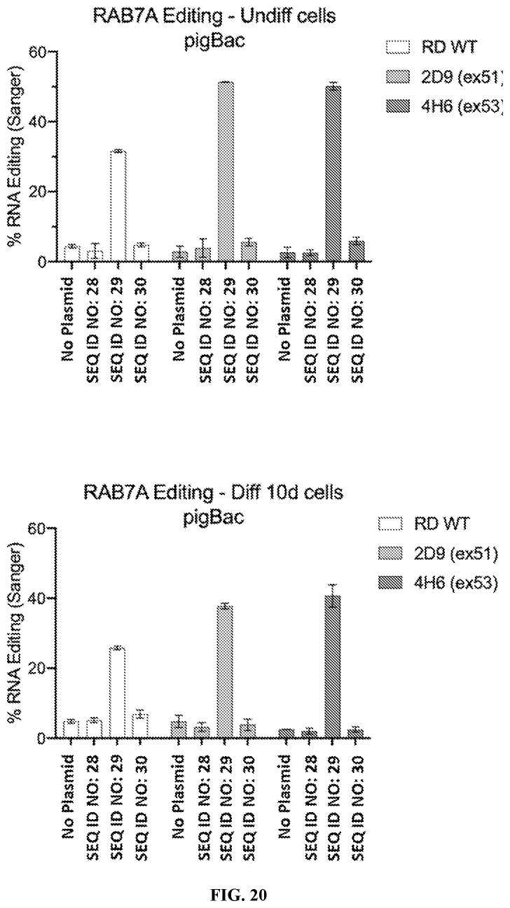

This example describes constructs of the present disclosure encoding for guide RNAs under the control of an hU1 promoter with a 5′ double hnRNP A1 binding domain, a SmOPT sequence, and a 3′ SmOPT hU7 hairpin, wherein the guide RNA is designed to target the 3′UTR of RAB7A to facilitate ADAR-mediated editing of an adenosine at the 3′UTR that immediately follows the translation termination codon. The guides used were guides of SEQ ID NO: 28-SEQ ID NO: 30. Editing was assessed in muscle cells. Rhabdomyosarcoma cells (RD WT), 2D9 cells (RD cells with a mutation hardwired into exon 51 splice site), and 4H6 cells (RD cells with a mutation hardwired into exon 53 splice site) were stably transfected with guide RNAs encoded for RAB7A 3′UTR in a piggyBac vector. Guide RNAs are described in the table below and expression was driven under the control of a human U1 promoter. No plasmid was used as a negative control. FIG. 20 shows editing of RAB7A using the guide RNA of SEQ ID NO: 28-SEQ ID NO: 30. Percent RNA editing was assessed in undifferentiated cells at 48 hours post plating and at 10 days post cell differentiation.

In all cells and in both timepoints, the linear guide RNAs of 100 bases in length and an A/C mismatch with the U7 hairpin and SmOPT sequence facilitated the highest levels of RAB7A 3′UTR editing by endogenous ADAR.

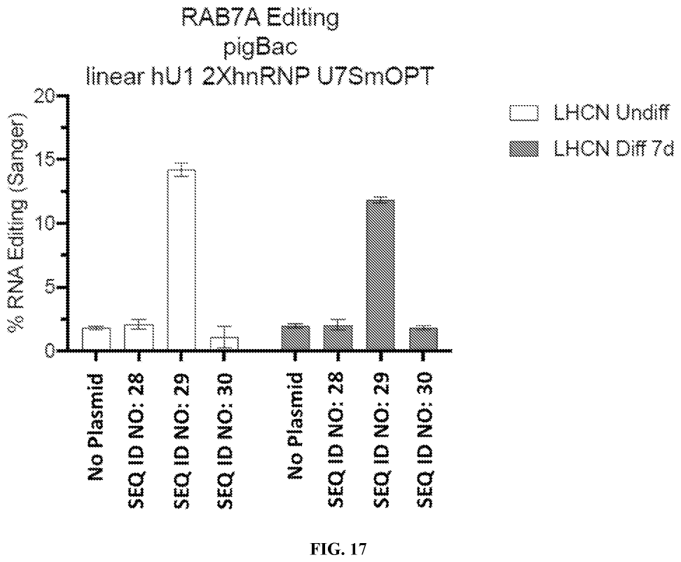

Each guide was then screened in an immortalized skeletal myoblast line LHCN-M2. As depicted in FIG. 17, guides tested showed sustained editing through 7 days of differentiation similar to the RD cells recited above.

### Example 14: RAB7A Editing with Guide RNAs

This example describes RAB7A editing with guide RNAs of the present disclosure in iPSC-derived neural cells, where the guide RNAs were delivered via AAV. iPSCs were induced to a neural lineage using dual SMAD inhibition for at least 6 days before infection with AAV2/2 expressing GFP (control) or Rab7 guides with or without a 3′ SmOPT hU7 hairpin. Cells were generally plated at 1*10\hat{ }5 cells per well in a 24 well plate and infected at the specified vg/cell. mRNA was isolated 48 hours, 72 hours, or 7 days post infection and Rab7 editing was assessed by ddPCR and Sanger sequencing. Transduction efficiency was assessed by quantifying images using ImageJ. Briefly, images were taken of each well that captured the GFP+ signal as well as a brightfield image of the same field of view. The GFP+ signal was thresholded and the area of positive signal was measured. Similarly, the area of the brightfield image that contained cells was thresholded and the area of positive signal was measured. The GFP+ area was divided by the cellular area to get percent transduction.

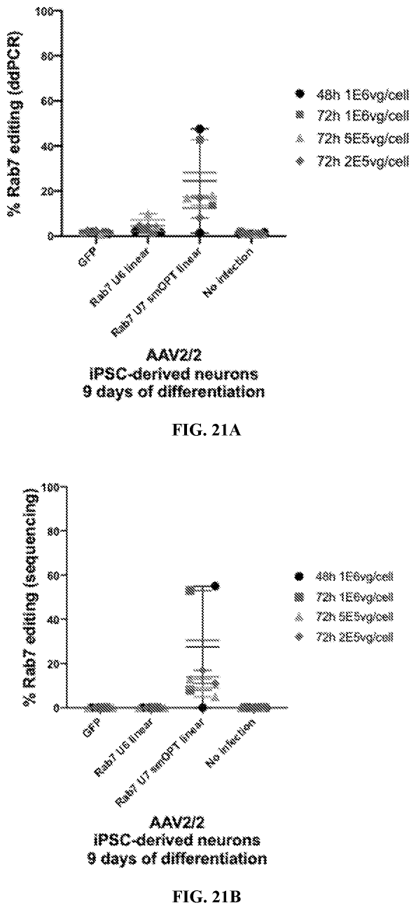

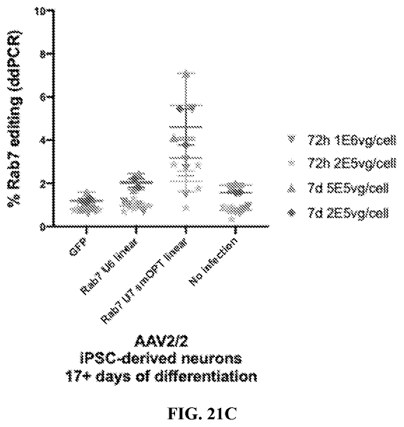

iPSCs were transduced with varying vg/cell for 48 or 72 h and were harvested at 8 or 9 days of differentiation. Rab7 editing was measured by ddPCR. As shown in FIG. 21A, there is a dose dependent increase in Rab7 editing as the vg/cell increases. Data represent biological replicates, mean+/−SEM. Rab7 editing was also measured by Sanger sequencing. As shown in FIG. 21B, there is a dose dependent increase in Rab7 editing as the vg/cell increases. The ddPCR and Sanger sequencing approaches to quantify sequencing produce very similar results. Data represent biological replicates, mean+/−SEM. For FIG. 21C, iPSCs were transduced with varying vg/cell for 72 h or 7 days and were harvested at 17 or more days of differentiation. Rab7 editing was measured by ddPCR. Data represent biological replicates, mean+/−SEM.

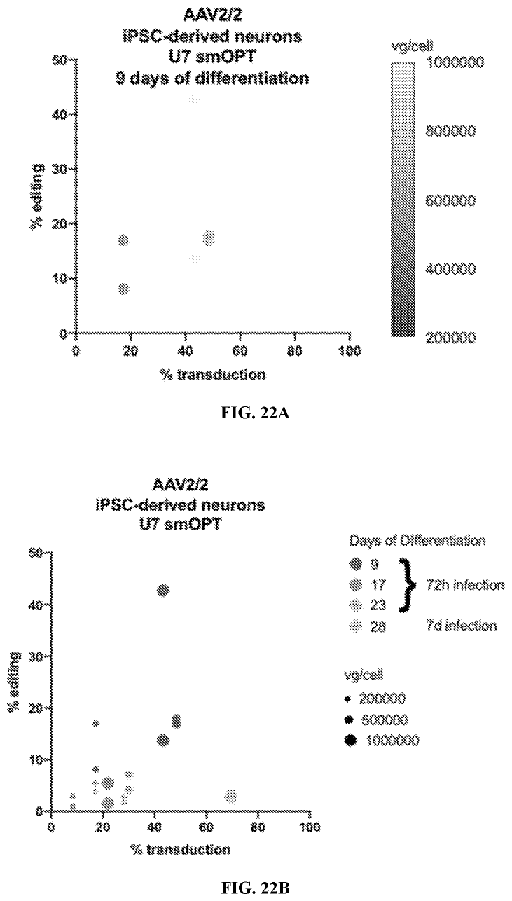

FIG. 22A depicts the % transduction plotted against the Rab7 editing efficiency in cells harvested after 9 days of differentiation, 72 h post infection. The colors of the dots represent the titer of virus that the cells were treated with. The viral titer is directly linked to transduction efficiency. FIG. 22B depicts the % transduction plotted against the Rab7 editing efficiency in cells harvested after varying days of differentiation. The colors of the dots represent the days of differentiation and the size of the dot represents the titer of virus that the cells were treated with.

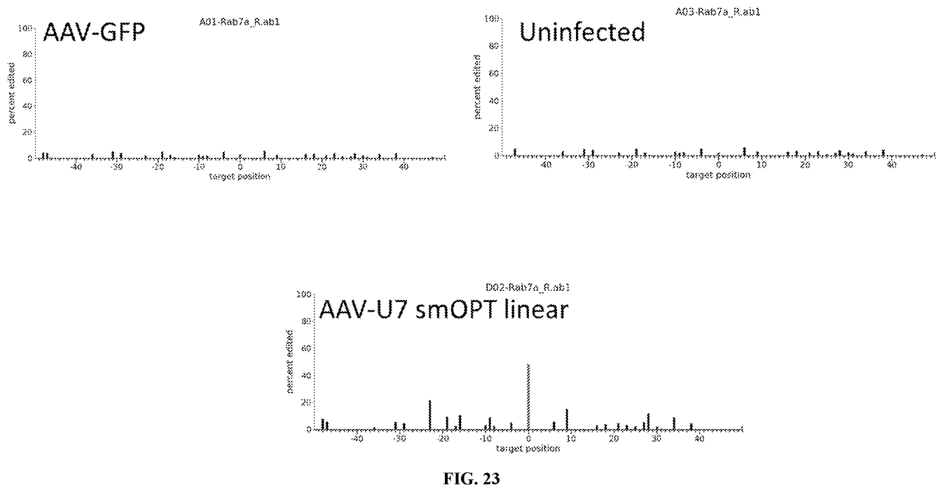

FIG. 23 shows off-target editing profiles for the U7 smOPT linear guide relative to control.

Table 3 below shows various sequences of the present disclosure. Formatting of elements in the sequences are in accordance with formatting in the second column of Table 3.

TABLE 3
Sequences of Engineered Nucleotides and Components of Engineered
Nucleotides According to some Embodiments Herein

| | | Sequence (where T is present, |
|---|---|---|
| | | sequences are represented as |
| | | DNA sequences) |
| | | K = G or T/U; Y = C or T/U; |
| SEQ ID NO | Name | R = G or A |
| SEQ ID NO: 1 | Mouse U7 promoter/ | TTAACAACATAGGAGCTGTGATTGGCTGTTT |
| | RAB7A 3′UTR | TCAGCCAATCAGCACTGACTCATTTGCATAG |
| | antisense/SmOPT | CCTTTACAAGCGGTCACAAACTCAAGAAACG |
| | mU7 hairpin.mU7 | AGCGGTTTTAATAGTCTTTTAGAATATTGTTT |
| | terminator | ATCGAACCGAATAAGGAACTGTGCTTTGTGA |
| | | TTCACATATCAGTGGAGGGGTGTGGAAATGG |
| | | CACCTTGATCTCACCCTCATCGAAAGTGGAG |
| | | TTGATGTCCTTCCCTGGCTCGCTACAGACGCA |
| | | CTTCCGCAccgTGATAAAAGGCGTACATAATT |
| | | CTTGTGTCTACTGTACAGAATACTGCCGCCA |
| | | GCTGGATTTCCCAATTCTGAGTAACACTCTGC |
| | | AATCCAAACAGGGTTCgtggAATTTTTGGAG |
| | | CAGGTTTTCTGACTTCGGTCGGAAAACCCCT |
| | | CCCAATTTCACTGGTCTACAATGAAAGCAAA |
| | | ACAGTTCTCTTCCCCGCTCCCCGGTGTGTGAG |
| | | AGGGGCTTTGATCCTTCTCTGGTTTCCTAGGA |
| | | AACGCGTATGTG |
| SEQ ID NO: 2 | Human U7 promoter/ | TTAACAACAACGAAGGGGCTGTGACTGGCTG |
| | RAB7A 3′UTR | CTTTCTCAACCAATCAGCACCGAACTCATTTG |
| | antisense/SmOPT | CATGGGCTGAGAACAAATGTTCGCGAACTCT |
| | hU7 hairpin.hU7 | AGAAATGAATGACTTAAGTAAGTTCCTTAGA |
| | terminator | ATATTATTTTTCCTACTGAAAGTTACCACATG |
| | | CGTCGTTGTTTATACAGTAATAGGAACAAGA |
| | | AAAAAGTCACCTAAGCTCACCCTCATCAATT |
| | | GTGGAGTTCCTTTATATCCCATCTTCTCTCCA |
| | | AACACATACGCAGAccgTGATAAAAGGCGTA |
| | | CATAATTCTTGTGTCTACTGTACAGAATACTG |
| | | CCGCCAGCTGGATTTCCCAATTCTGAGTAAC |
| | | ACTCTGCAATCCAAACAGGGTTCgtggAATTTT |
| | | TGGAGTAGGCTTTCTGGCTTTTTACCGGAAA |
| | | GCCCCTCTTATGATGTTTGTTGCCAATGATAG |
| | | ATTGTTTTCACTGTGCAAAAATTATGGGTAGT |
| | | TTTGGTGGTCTTGATGCAGTTGTAAGCTTGGG |
| | | GTATG |
| SEQ ID NO: 3 | Human U1 promoter/ | TAAGGACCAGCTTCTTTGGGAGAGAACAGAC |
| | RAB7A 3′UTR | GCAGGGGCGGGAGGGAAAAAGGGAGAGGCA |
| | antisense/hU1 | GACGTCACTTCCTCTTGGCGACTCTGGCAGC |
| | hairpin.hU1 | AGATTGGTCGGTTGAGTGGCAGAAAGGCAG |
| | terminator | ACGGGGACTGGGCAAGGCACTGTCGGTGAC |
| | | ATCACGGACAGGGCGACTTCTATGTAGATGA |
| | | GGCAGCGCAGAGGCTGCTGCTTCGCCACTTG |
| | | CTGCTTCGCCACGAAGGGAGTTCCCGTGCCC |
| | | TGGGAGCGGGTTCAGGACCGCTGATCGGAAG |
| | | TGAGAATCCCAGCTGTGTGTCAGGGCTGGAA |
| | | AGGGCTCGGGAGTGCGCGGGGCAAGTGACC |
| | | GTGTGTGTAAAGAGTGAGGCGTATGAGGCTG |
| | | TGTCGGGGCAGAGCCCGAAGATCTCaccgTGA |
| | | TAAAAGGCGTACATAATTCTTGTGTCTACTGT |
| | | ACAGAATACTGCCGCCAGCTGGATTTCCCAA |
| | | TTCTGAGTAACACTCTGCAATCCAAACAGGG |
| | | TTCgtGGCAGGGGAGATACCATGATCACGAA |
| | | GGTGGTTTTCCCAGGGCGAGGCTTATCCATT |
| | | GCACTCCGGATGTGCTGACCCCTGCGATTTC |
| | | CCCAAATGTGGGAAACTCGACTGCATAATTT |
| | | GTGGTAGTGGGGGACTGCGTTCGCGCTTTCC |
| | | CCTGACTTTCTGGAGTTTCAAAAACAGACTG |
| | | TACGCCAAGGGTCATATC |
| SEQ ID NO: 4 | Human U1 promoter/ | TAAGGACCAGCTTCTTTGGGAGAGAACAGAC |
| | RAB7A 3′UTR | GCAGGGGCGGGAGGGAAAAAGGGAGAGGCA |
| | antisense/SmOPT | GACGTCACTTCCTCTTGGCGACTCTGGCAGC |
| | mU7 hairpin.mU7 | AGATTGGTCGGTTGAGTGGCAGAAAGGCAG |
| | terminator | ACGGGGACTGGGCAAGGCACTGTCGGTGAC |
| | | ATCACGGACAGGGCGACTTCTATGTAGATGA |
| | | GGCAGCGCAGAGGCTGCTGCTTCGCCACTTG |
| | | CTGCTTCGCCACGAAGGGAGTTCCCGTGCCC |
| | | TGGGAGCGGGTTCAGGACCGCTGATCGGAAG |
| | | TGAGAATCCCAGCTGTGTGTCAGGGCTGGAA |
| | | AGGGCTCGGGAGTGCGCGGGGCAAGTGACC |
| | | GTGTGTGTAAAGAGTGAGGCGTATGAGGCTG |
| | | TGTCGGGGCAGAGCCCGAAGATCTCaccgTGA |
| | | TAAAAGGCGTACATAATTCTTGTGTCTACTGT |
| | | ACAGAATACTGCCGCCAGCTGGATTTCCCAA |
| | | TTCTGAGTAACACTCTGCAATCCAAACAGGG |
| | | TTCgtggAATTTTTGGAGCAGGTTTTCTGACTT |
| | | CGGTCGGAAAACCCCTCCCAATTTCACTGGT |
| | | CTACAATGAAAGCAAAACAGTTCTCTTCCCC |
| | | GCTCCCCGGTGTGTGAGAGGGGCTTTGATCC |
| | | TTCTCTGGTTTCCTAGGAAACGCGTATGTG |
| SEQ ID NO: 5 | Mouse U7 promoter/ | TTAACAACATAGGAGCTGTGATTGGCTGTTT |
| | RAB7A exon1 | TCAGCCAATCAGCACTGACTCATTTGCATAG |
| | antisense/SmOPT | CCTTTACAAGCGGTCACAAACTCAAGAAACG |
| | mU7 hairpin.mU7 | AGCGGTTTTAATAGTCTTTTAGAATATTGTTT |
| | terminator | ATCGAACCGAATAAGGAACTGTGCTTTGTGA |
| | | TTCACATATCAGTGGAGGGGTGTGGAAATGG |
| | | CACCTTGATCTCACCCTCATCGAAAGTGGAG |
| | | TTGATGTCCTTCCCTGGCTCGCTACAGACGCA |
| | | CTTCCGCAccGGGGGCTCCGGGCCGGGCGCGT |
| | | CGCGAGGGCTCCCGCCGAGGAGGAGACC |
| | | AAACGGAGGACAGAAGCGAGAAGGTCCAAG |
| | | TTCTGGTTCCAGGGAACTCTgtggAATTTTTGG |
| | | AGCAGGTTTTCTGACTTCGGTCGGAAAACCC |
| | | CTCCCAATTTCACTGGTCTACAATGAAAGCA |
| | | AAACAGTTCTCTTCCCCGCTCCCCGGTGTGTG |
| | | AGAGGGGCTTTGATCCTTCTCTGGTTTCCTAG |
| | | GAAACGCGTATGTG |
| SEQ ID NO: 6 | Human U7 promoter/ | TTAACAACAACGAAGGGGCTGTGACTGGCTG |
| | RAB7A exon3 | CTTTCTCAACCAATCAGCACCGAACTCATTTG |
| | antisense/SmOPT | CATGGGCTGAGAACAAATGTTCGCGAACTCT |
| | hU7 hairpin.hU7 | AGAAATGAATGACTTAAGTAAGTTCCTTAGA |
| | terminator | ATATTATTTTTCCTACTGAAAGTTACCACATG |
| | | CGTCGTTGTTTATACAGTAATAGGAACAAGA |
| | | AAAAAGTCACCTAAGCTCACCCTCATCAATT |
| | | GTGGAGTTCCTTTATATCCCATCTTCTCTCCA |
| | | AACACATACGCAGAccgTGACTAGCCTGTCAT |
| | | CCACCATCACCTCCTTGGTCAGAAAGTCAGC |
| | | TCCCATTGTGGCTTTGTACTGATTGCTGAATT |
| | | TCTTATTCACATACTGGTTCATgtggAATTTTTG |
| | | GAGTAGGCTTTCTGGCTTTTTACCGGAAAGC |
| | | CCCTCTTATGATGTTTGTTGCCAATGATAGAT |
| | | TGTTTTCACTGTGCAAAAATTATGGGTAGTTT |
| | | TGGTGGTCTTGATGCAGTTGTAAGCTTGGGG |
| | | TATG |
| SEQ ID NO: 7 | Mouse U7 promoter/ | TTAACAACATAGGAGCTGTGATTGGCTGTTT |
| | SNCA start codon | TCAGCCAATCAGCACTGACTCATTTGCATAG |
| | antisense/SmOPT | CCTTTACAAGCGGTCACAAACTCAAGAAACG |
| | mU7 hairpin.mU7 | AGCGGTTTTAATAGTCTTTTAGAATATTGTTT |
| | terminator | ATCGAACCGAATAAGGAACTGTGCTTTGTGA |
| | | TTCACATATCAGTGGAGGGGTGTGGAAATGG |
| | | CACCTTGATCTCACCCTCATCGAAAGTGGAG |
| | | TTGATGTCCTTCCCTGGCTCGCTACAGACGCA |
| | | CTTCCGCAccgGCCACAACTCCCTCCTTGGCCT |
| | | TTGAAAGTCCTTTCATGAATACATCCACGGC |
| | | TAATGAATTCCTTTACACCACACTGTCGTCGA |
| | | ATGGCCACTCCCAGTgtggAATTTTTGGAGCAG |
| | | GTTTTCTGACTTCGGTCGGAAAACCCCTCCCA |
| | | ATTTCACTGGTCTACAATGAAAGCAAAACAG |
| | | TTCTCTTCCCCGCTCCCCGGTGTGTGAGAGGG |
| | | GCTTTGATCCTTCTCTGGTTTCCTAGGAAACG |
| | | CGTATGTG |
| SEQ ID NO: 8 | Mouse U7 promoter/ | TTAACAACATAGGAGCTGTGATTGGCTGTTT |
| | SNCA 3′UTR | TCAGCCAATCAGCACTGACTCATTTGCATAG |
| | antisense/SmOPT | CCTTTACAAGCGGTCACAAACTCAAGAAACG |
| | mU7 hairpin.mU7 | AGCGGTTTTAATAGTCTTTTAGAATATTGTTT |
| | terminator | ATCGAACCGAATAAGGAACTGTGCTTTGTGA |
| | | TTCACATATCAGTGGAGGGGTGTGGAAATGG |
| | | CACCTTGATCTCACCCTCATCGAAAGTGGAG |
| | | TTGATGTCCTTCCCTGGCTCGCTACAGACGCA |
| | | CTTCCGCAccgAACATCGTAGATTGAAGCCAC |
| | | AAAATCCACAGCACACAAAGACCCTGCCACC |
| | | ATGTATTCACTTCAGTGAAAGGGAAGCACCG |
| | | AAATGCTGAGTGGGGGCgtggAATTTTTGGAG |
| | | CAGGTTTTCTGACTTCGGTCGGAAAACCCCT |
| | | CCCAATTTCACTGGTCTACAATGAAAGCAAA |
| | | ACAGTTCTCTTCCCCGCTCCCCGGTGTGTGAG |
| | | AGGGGCTTTGATCCTTCTCTGGTTTCCTAGGA |
| | | AACGCGTATGTG |
| SEQ ID NO: 9 | RAB7A3′UTR | TGATAAAAGGCGTACATAATTCTTGTGTCTA |
| | antisense | CTGTACAGAATACTGCCGCCAGCTGGATTTC |
| | | CCAATTCTGAGTAACACTCTGCAATCCAAAC |
| | | AGGGTTC |
| SEQ ID NO: 10 | RAB7A exon 1 | GGGGGCTCCGGGCCGGGCGCGTCGCGAGGG |
| | antisense | CTCCCGCCGAGGAGGAGACCAAACGGAGGA |
| | | CAGAAGCGAGAAGGTCCAAGTTCTGGTTCCA |
| | | GGGAACTCT |
| SEQ ID NO: 11 | RAB7A exon 3 | TGACTAGCCTGTCATCCACCATCACCTCCTTG |
| | antisense | GTCAGAAAGTCAGCTCCCATTGTGGCTTTGT |
| | | ACTGATTGCTGAATTTCTTATTCACATACTGG |
| | | TTCAT |
| SEQ ID NO: 12 | RAB7A exon 4 | TTTCAGGATCTCGGGGACTGGCCTGGATGAG |
| | antisense | AAACTCATCTCTCCAGCCATCTAGGGTTTGG |
| | | AATGTGTTGGGGGCAGTCACATCAAATACCA |
| | | GAACGC |
| SEQ ID NO: 13 | DMD exon 71 | AAGTACTCACGCAGAATCTACTGGCCAGAAG |
| | antisense | TTGATCAGAGTAACGGGACCGCAAAACAAA |
| | | AAATGAGGTGGTGAAGGAGACACACGCAAA |
| | | CTCAGCCGC |
| SEQ ID NO: 14 | DMD exon 74 | CTGGTTCAAACTTTGGCAGTAATGCTGGATT |
| | antisense | AACAAATGTTCATCATCTCCGGAAAATAAAA |
| | | TCAAAGGTTGTGGTTTGTTCCCCCCCTTATGT |
| | | TGCTTT |
| SEQ ID NO: 15 | SNCA start codon | GCCACAACTCCCTCCTTGGCCTTTGAAAGTCC |
| | antisense | TTTCATGAATACATCCACGGCTAATGAATTC |
| | | CTTTACACCACACTGTCGTCGAATGGCCACT |
| | | CCCAGT |
| SEQ ID NO: 16 | SNCA3′UTR | AACATCGTAGATTGAAGCCACAAAATCCACA |
| | antisense | GCACACAAAGACCCTGCCACCATGTATTCAC |
| | | TTCAGTGAAAGGGAAGCACCGAAATGCTGA |
| | | GTGGGGGC |
| SEQ ID NO: 17 | SNCA exon 4 | AGCCAGTGGCTGCTGCAATGCTCCCTGCTCC |
| | antisense | CTCCACTGTCTTCTGGGCCACTGCTGTCACAC |
| | | CCGTCACCACTGCTCCTCCAACATTTGTCACT |
| | | TGCTC |
| SEQ ID NO: 18 | LRRK2 V0118 | GTGCCCTCTGATGTTTTTATCCCCATTCTACA |
| | SmOPT | GCATTACGGAGCAGTGCCGTAGTGTCGTTTC |
| | mU7 hairpin | TTTGCAATGATGGCAGCATTGGGATACAGTG |
| | | TGAAAAgtggAATTTTTGGAGCAGGTTTTCTGA |
| | | CTTCGGTCGGAAAACCCCT |
| SEQ ID NO: 19 | LRRK2 V0118 | CTGGCAACTTCAGGTGCACGAAACCCTGGTG |
| | SmOPT | TGCCCTCTGATGTTTTTATCCCCATTCTACAG |
| | mU7 hairpin | CATTACGGAGCAGTGCCGTAGTGTCGTTTCTT |
| | | TGCAAgtggAATTTTTGGAGCAGGTTTTCTGAC |
| | | TTCGGTCGGAAAACCCCT |
| SEQ ID NO: 20 | ABCA4 U6 Full | GCCGGCACCATTCACTCCCAGGAGGCCAAAG |
| | mimicry | CACTCTCCAGTGAGAACTCGGACCACAGCCT |
| | | CCCGCTGCTGGGCTGGAGGTGCCTGGATAAA |
| | | TCTTGGT |
| SEQ ID NO: 21 | ABCA4 U6 Full | CCCCAGTGAGCATCTTGAATGTGGTTGTATT |
| | mimicry | GCCGGCACCATTCACTCCCAGGAGGCCAAAG |
| | | CACTCTCCAGTGAGAACTCGGACCACAGCCT |
| | | CCCGCTG |
| SEQ ID NO: 22 | ABCA4 U1 SmOPT | CCCCAGTGAGCATCTTGAATGTGGTTGTATT |
| | Full mimicry | GCCGGCACCATTCACTCCCAGGAGGCCAAAG |
| | SmOPT | CACTCTCCAGTGAGAACTCGGACCACAGCCT |
| | mU7 hairpin | CCCGCTGgtggAATTTTTGGAGCAGGTTTTCTG |
| | | ACTTCGGTCGGAAAACCCCT |
| SEQ ID NO: 23 | CAPS1 v0030 U1 | CCCAGTGAGCATCTTGAATGTGGTTGTTTTGC |
| | SmOPT | CGGCACCATTCACTCCCAGGAGGCCAAAGCA |
| | SmOPT | CGCTCCAGGGCGAACTTATCACATACAGCCT |
| | mU7 hairpin | GTCCACgtggAATTTTTGGAGCAGGTTTTCTGA |
| | | CTTCGGTCGGAAAACCCCT |
| SEQ ID NO: 24 | SOD1 start codon | GCCCTGCACTGGGCCGTCGCCCTTCAGCACG |
| | antisense | CACACGGCCTTCGTCGCCACAACTCGCTAGG |
| | | CCACGCCGAGGTCCTGGTTCCGAGGACTGCA |
| | | ACGGAAA |
| SEQ ID NO: 25 | hnRNPA1/SOD1 start | TATGATAGGGACTTAGGGTGGCCCTGCACTG |
| | codon antisense | GGCCGTCGCCCTTCAGCACGCACACGGCCTT |
| | | CGTCGCCACAACTCGCTAGGCCACGCCGAGG |
| | | TCCTGGTTCCGAGGACTGCAACGGAAA |
| SEQ ID NO: 26 | SERPINA1 guide | accgAUGGGUAUGGCCUCUAAAAACAUGGCC |
| | | CCAGCAGCUUCAGUCCCUUUCUCGUCGAUG |
| | | GUCAGCACAGCCUUAUGCACGGCCUGGAGG |
| | | GGAGAGAAGCAGAguggAAUUUUUGGAG |
| | | CAGGUUUUCUGACUUCGGUCGGAAAACCCC |
| | | U |
| SEQ ID NO: 27 | SERPINA1 guide | accgAUGGGUAUGGCCUCUAAAAACAUGGCC |
| | | CCAGCAGCUUCAGUCCCUUACUCGUCGAUG |
| | | GUCAGCACAGCCUUAUGCACGGCCUGGAGG |
| | | GGAGAGAAGCAGAguggAAUUUUUGGAG |
| | | CAGGUUUUCUGACUUCGGUCGGAAAACCCC |
| | | U |
| SEQ ID NO: 28 | Backbone Only (no | TAAGGACCAGCTTCTTTGGGAGAGAACAGAC |
| | guide, only hU1 | GCAGGGGCGGGAGGGAAAAAGGGAGAGGCA |
| | promoter and smOPT | GACGTCACTTCCTCTTGGCGACTCTGGCAGC |
| | mU7 hairpin.mU7 | AGATTGGTCGGTTGAGTGGCAGAAAGGCAG |
| | terminator | ACGGGGACTGGGCAAGGCACTGTCGGTGAC |
| | | ATCACGGACAGGGCGACTTCTATGTAGATGA |
| | | GGCAGCGCAGAGGCTGCTGCTTCGCCACTTG |
| | | CTGCTTCGCCACGAAGGGAGTTCCCGTGCCC |
| | | TGGGAGCGGGTTCAGGACCGCTGATCGGAAG |
| | | TGAGAATCCCAGCTGTGTGTCAGGGCTGGAA |
| | | AGGGCTCGGGAGTGCGCGGGGCAAGTGACC |
| | | GTGTGTGTAAAGAGTGAGGCGTATGAGGCTG |
| | | TGTCGGGGCAGAGCCCGAAGATCTC |
| | | accgagagaccagctggatggtctcagtggAATTTTTGGAG |
| | | CAGGTTTTCTGACTTCGGTCGGAAAACCCCT |
| | | CCCAATTTCACTGGTCTACAATGAAAGCAAA |
| | | ACAGTTCTCTTCCCCGCTCCCCGGTGTGTGAG |
| | | AGGGGCTTTGATCCTTCTCTGGTTTCCTAGGA |
| | | AACGCGTATGTG |
| SEQ ID NO: 29 | hU1 promoter | TAAGGACCAGCTTCTTTGGGAGAGAACAGAC |
| | 2xhnRNP linear | GCAGGGGCGGGAGGGAAAAAGGGAGAGGCA |
| | RAB7A 3′UTR | GACGTCACTTCCTCTTGGCGACTCTGGCAGC |
| | smOPT U7 | AGATTGGTCGGTTGAGTGGCAGAAAGGCAG |
| | hairpin.mU7 | ACGGGGACTGGGCAAGGCACTGTCGGTGAC |
| | terminator | ATCACGGACAGGGCGACTTCTATGTAGATGA |
| | | GGCAGCGCAGAGGCTGCTGCTTCGCCACTTG |
| | | CTGCTTCGCCACGAAGGGAGTTCCCGTGCCC |
| | | TGGGAGCGGGTTCAGGACCGCTGATCGGAAG |
| | | TGAGAATCCCAGCTGTGTGTCAGGGCTGGAA |
| | | AGGGCTCGGGAGTGCGCGGGGCAAGTGACC |
| | | GTGTGTGTAAAGAGTGAGGCGTATGAGGCTG |
| | | TGTCGGGGCAGAGCCCGAAGATCTCaccgTAT |
| | | GATAGGGACTTAGGGTGTGATAAAAGGCGTA |
| | | CATAATTCTTGTGTCTACTGTACAGAATACTG |
| | | CCGCCAGCTGGATTTCCCAATTCTGAGTAAC |
| | | ACTCTGCAATCCAAACAGGGTTCgtggAATTTT |
| | | TGGAGCAGGTTTTCTGACTTCGGTCGGAAAA |
| | | CCCCTCCCAATTTCACTGGTCTACAATGAAA |
| | | GCAAAACAGTTCTCTTCCCCGCTCCCCGGTGT |
| | | GTGAGAGGGGCTTTGATCCTTCTCTGGTTTCC |
| | | TAGGAAACGCGTATGTG |
| SEQ ID NO: 30 | hU1 promoter | TAAGGACCAGCTTCTTTGGGAGAGAACAGAC |
| | 2xhnRNP linear | GCAGGGGCGGGAGGGAAAAAGGGAGAGGCA |
| | RAB7A 3′UTR | GACGTCACTTCCTCTTGGCGACTCTGGCAGC |
| | smOPT mU7 | AGATTGGTCGGTTGAGTGGCAGAAAGGCAG |
| | hairpin.mU7 | ACGGGGACTGGGCAAGGCACTGTCGGTGAC |
| | terminator | ATCACGGACAGGGCGACTTCTATGTAGATGA |
| | | GGCAGCGCAGAGGCTGCTGCTTCGCCACTTG |
| | | CTGCTTCGCCACGAAGGGAGTTCCCGTGCCC |
| | | TGGGAGCGGGTTCAGGACCGCTGATCGGAAG |
| | | TGAGAATCCCAGCTGTGTGTCAGGGCTGGAA |
| | | AGGGCTCGGGAGTGCGCGGGGCAAGTGACC |
| | | GTGTGTGTAAAGAGTGAGGCGTATGAGGCTG |
| | | TGTCGGGGCAGAGCCCGAAGATCTCaccgTAT |
| | | GATAGGGACTTAGGGTGTCTTGTGTCTACTG |
| | | TACAGAATACTGCCGCCAGCTGGATTTCCCA |
| | | ATTCTGAGTAACACTCTGCAATCCAAACAGG |
| | | GTTCAACCCTCCACCTTACAGGCCTGCATTAC |
| | | AGGACTTAAACACATAATCCAAGAATTTCTT |
| | | ACACTAATTTATACATTTTAATTGGTTGCATA |
| | | TATTAACATGTACTATAAGATTCTTTTCTgtgg |
| | | AATTTTTGGAGCAGGTTTTCTGACTTCGGTCG |
| | | GAAAACCCCTCCCAATTTCACTGGTCTACAA |
| | | TGAAAGCAAAACAGTTCTCTTCCCCGCTCCC |
| | | CGGTGTGTGAGAGGGGCTTTGATCCTTCTCT |
| | | GGTTTCCTAGGAAACGCGTATGTG |
| SEQ ID NO: 31 | SmOPT | TAATCTTTGAAAGTCCTTTCATGAATACATCC |
| | mU7 hairpin | ACGGCTAATGAATTCCTTTACACCACATTAG |
| | | CCAGAAGGCTTGAAGGCAAGGCGTGAGGGA |
| | | GCGCCCAGGACGCTCTCGGAG)TATATAAATT |
| | | TTTGGAGCAGGTTTTCTGACTTCGGTCGGAA |
| | | AACCCCT |
| SEQ ID NO: 32 | SmOPT | TAATCTTTGAAAGTCCTTTCATGAATACATCC |
| | mU7 hairpin | ACGGCTAATGAATTCCTTTACACCACAAGGG |
| | | GCGAATGGCCACTCCCAGTTCTCCGCTCACG |
| | | AGGGTGGAAATAATTAAGGCGTGAGGGAGC |
| | | GCCCAGGACGCTCTCATATATAAATTTTTGG |
| | | AGCAGGTTTTCTGACTTCGGTCGGAAAACCC |
| | | CT |
| SEQ ID NO: 33 | SmOPT | TAATTTTCTCAGCAGCAGCCACAACTCCGAG |
| | mU7 hairpin | GAACCCCTTTGAAAGTCCTTTCATGAATACA |
| | | TCCACGGCTAAACTTCTCCTTTACACCACACT |
| | | GTCGTCGAATGGCCACTCCCAGTATATATAA |
| | | ATTTTTGGAGCAGGTTTTCTGACTTCGGTCGG |
| | | AAAACCCCT |
| SEQ ID NO: 34 | Human U6 promoter | GAGGGCCTATTTCCCATGATTCCTTCATATTT |
| | | GCATATACGATACAAGGCTGTTAGAGAGATA |
| | | ATTAGAATTAATTTGACTGTAAACACAAAGA |
| | | TATTAGTACAAAATACGTGACGTAGAAAGTA |
| | | ATAATTTCTTGGGTAGTTTGCAGTTTTAAAAT |
| | | TATGTTTTAAAATGGACTATCATATGCTTACC |
| | | GTAACTTGAAAGTATTTCGATTTCTTGGCTTT |
| | | ATATATCTTGTGGAAAGGACGAAACACC |
| SEQ ID NO: 35 | Mouse U6 promoter | GTACTGAGTCGCCCAGTCTCAGATAGATCCG |
| | | ACGCCGCCATCTCTAGGCCCGCGCCGGCCCC |
| | | CTCGCACAGACTTGTGGGAGAAGCTCGGCTA |
| | | CTCCCCTGCCCCGGTTAATTTGCATATAATAT |
| | | TTCCTAGTAACTATAGAGGCTTAATGTGCGA |
| | | TAAAAGACAGATAATCTGTTCTTTTTAATACT |
| | | AGCTACATTTTACATGATAGGCTTGGATTTCT |
| | | ATAAGAGATACAAATACTAAATTATTATTTT |
| | | AAAAAACAGCACAAAAGGAAACTCACCCTA |
| | | ACTGTAAAGTAATTGTGTGTTTTGAGACTAT |
| | | AAATATCCCTTGGAGAAAAGCCTTGTTTG |
| SEQ ID NO: 36 | Human U7 promoter | TTAACAACAACGAAGGGGCTGTGACTGGCTG |
| | | CTTTCTCAACCAATCAGCACCGAACTCATTTG |
| | | CATGGGCTGAGAACAAATGTTCGCGAACTCT |
| | | AGAAATGAATGACTTAAGTAAGTTCCTTAGA |
| | | ATATTATTTTTCCTACTGAAAGTTACCACATG |
| | | CGTCGTTGTTTATACAGTAATAGGAACAAGA |
| | | AAAAAGTCACCTAAGCTCACCCTCATCAATT |
| | | GTGGAGTTCCTTTATATCCCATCTTCTCTCCA |
| | | AACACATACGCA |
| SEQ ID NO: 37 | Human U7 promoter | TTAACAACAACGAAGGGGCTGTGACTGGCTG |
| | | CTTTCTCAACCAATCAGCACCGAACTCATTTG |
| | | CATGGGCTGAGAACAAATGTTCGCGAACTCT |
| | | AGAAATGAATGACTTAAGTAAGTTCCTTAGA |
| | | ATATTATTTTTCCTACTGAAAGTTACCACATG |
| | | CGTCGTTGTTTATACAGTAATAGGAACAAGA |
| | | AAAAAGTCACCTAAGCTCACCCTCATCAATT |
| | | GTGGAGTTCCTTTATATCCCATCTTCTCTCCA |
| | | AACACATACGCAG |
| SEQ ID NO: 38 | Mouse U7 promoter | TTAACAACATAGGAGCTGTGATTGGCTGTTT |
| | | TCAGCCAATCAGCACTGACTCATTTGCATAG |
| | | CCTTTACAAGCGGTCACAAACTCAAGAAACG |
| | | AGCGGTTTTAATAGTCTTTTAGAATATTGTTT |
| | | ATCGAACCGAATAAGGAACTGTGCTTTGTGA |
| | | TTCACATATCAGTGGAGGGGTGTGGAAATGG |
| | | CACCTTGATCTCACCCTCATCGAAAGTGGAG |
| | | TTGATGTCCTTCCCTGGCTCGCTACAGACGCA |
| | | CTTCCGC |
| SEQ ID NO: 39 | Human U1 promoter | TAAGGACCAGCTTCTTTGGGAGAGAACAGAC |
| | | GCAGGGGCGGGAGGGAAAAAGGGAGAGGCA |
| | | GACGTCACTTCCTCTTGGCGACTCTGGCAGC |
| | | AGATTGGTCGGTTGAGTGGCAGAAAGGCAG |
| | | ACGGGGACTGGGCAAGGCACTGTCGGTGAC |
| | | ATCACGGACAGGGCGACTTCTATGTAGATGA |
| | | GGCAGCGCAGAGGCTGCTGCTTCGCCACTTG |
| | | CTGCTTCGCCACGAAGGGAGTTCCCGTGCCC |
| | | TGGGAGCGGGTTCAGGACCGCTGATCGGAAG |
| | | TGAGAATCCCAGCTGTGTGTCAGGGCTGGAA |
| | | AGGGCTCGGGAGTGCGCGGGGCAAGTGACC |
| | | GTGTGTGTAAAGAGTGAGGCGTATGAGGCTG |
| | | TGTCGGGGCAGAGCCCGAAGATCTC |
| SEQ ID NO: 40 | CMV promoter | ATACGCGTTGACATTGATTATTGACTAGTTAT |
| | | TAATAGTAATCAATTACGGGGTCATTAGTTC |
| | | ATAGCCCATATATGGAGTTCCGCGTTACATA |
| | | ACTTACGGTAAATGGCCCGCCTGGCTGACCG |
| | | CCCAACGACCCCCGCCCATTGACGTCAATAA |
| | | TGACGTATGTTCCCATAGTAACGCCAATAGG |
| | | GACTTTCCATTGACGTCAATGGGTGGAGTAT |
| | | TTACGGTAAACTGCCCACTTGGCAGTACATC |
| | | AAGTGTATCATATGCCAAGTACGCCCCCTAT |
| | | TGACGTCAATGACGGTAAATGGCCCGCCTGG |
| | | CATTATGCCCAGTACATGACCTTATGGGACT |
| | | TTCCTACTTGGCAGTACATCTACGTATTAGTC |
| | | ATCGCTATTACCATGGTGATGCGGTTTTGGC |
| | | AGTACATCAATGGGCGTGGATAGCGGTTTGA |
| | | CTCACGGGGATTTCCAAGTCTCCACCCCATT |
| | | GACGTCAATGGGAGTTTGTTTTGGCACCAAA |
| | | ATCAACGGGACTTTCCAAAATGTCGTAACAA |
| | | CTCCGCCCCATTGACGCAAATGGGCGGTAGG |
| | | CGTGTACGGTGGGAGGTCTATATAAGCAGAG |
| | | CTCGTTTAGTGAACCGTCAGATCGCCTGGAG |
| | | ACGCCATCCACGCTGTTTTGACCTCCATAGA |
| | | AGACACCGGGACCGATCCAGCCTCCGGACTC |
| | | TAGAGGATCGAACC |
| SEQ ID NO: 41 | SmOPT sequence | AATTTTTGGAG |
| SEQ ID NO: 42 | Mouse U7 Hairpin | CAGGTTTTCTGACTTCGGTCGGAAAACCCCT |
| | Sequence | |
| SEQ ID NO: 43 | Human U7 Hairpin | TAGGCTTTCTGGCTTTTTACCGGAAAGCCCCT |
| | Sequence | |
| SEQ ID NO: 44 | variant Sm binding | AATTTKTGGAGCAGGYTTTCTGACTTCGGTC |
| | domain and U7 hairpin | GGAAARCCCCT |
| | sequence | |
| SEQ ID NO: 45 | SmOPT mU7 hairpin. | AATTTTTGGAGCAGGTTTTCTGACTTCGGTCG |
| | mU7 terminator | GAAAACCCCTCCCAATTTCACTGGTCTACAA |
| | | TGAAAGCAAAACAGTTCTCTTCCCCGCTCCC |
| | | CGGTGTGTGAGAGGGGCTTTGATCCTTCTCT |
| | | GGTTTCCTAGGAAACGCGTATGTG |
| SEQ ID NO: 46 | Human U1 hairpin | GGCAGGGGAGATACCATGATCACGAAGGTG |
| | sequence | GTTYTCCCAGGGCGAGGCTTATCCATTGCAC |
| | | TCCGGATGTGCTGACCCCTGCGATTTCCCCA |
| | | AATGTGGGAAACTCGACTGCATAATTTGTGG |
| | | TAGTGGGGGACTGCGTTCGCGCTTTCCCCTG |
| SEQ ID NO: 47 | hnRNPA1 sequence 1 | TATGATAGGGACTTAGGGTG |
| SEQ ID NO: 48 | hnRNPA1 sequence 2 | TAGGGATAGGGATAGGGA |
| SEQ ID NO: 49 | GluR2 sequence | GUGGAAUAGUAUAACAAUAUGCUAAAUGU |
| | | UGUUAUAGUAUCCCAC |
| SEQ ID NO: 50 | spacer domain | ATATA |
| SEQ ID NO: 51 | spacer domain | ATAAT |
| SEQ ID NO: 52 | spacer domain | AUAAU |
| SEQ ID NO: 53 | spacer domain | AUAUA |
| SEQ ID NO: 54 | spacer domain | UAAUA |
| SEQ ID NO: 55 | spacer domain | AGTTTTTTTTA |
| SEQ ID NO: 56 | spacer domain | AGAAAAAAAATA |

What is claimed:

1. An engineered polynucleotide comprising:
a targeting sequence that at least partially hybridizes to at least a portion of a target RNA and contains at least one mismatch when at least partially hybridized to the portion of the target RNA;
an Sm or Sm-like protein binding domain, or variant thereof, from a spliceosomal snRNA or a non-spliceosomal small nuclear RNA (snRNA);
a hairpin from a spliceosomal snRNA or a non-spliceosomal snRNA, or a variant of either of these;
wherein the engineered polynucleotide is configured to facilitate editing of a base of the target RNA by an RNA editing entity.

2. An engineered polynucleotide comprising:
a targeting sequence that at least partially hybridizes to at least a portion of a target RNA and contains at least one mismatched nucleotide, wherein the target RNA comprises a mutation in an exon that is implicated in a disease or condition;
an Sm or Sm-like protein binding domain or variant thereof from a spliceosomal snRNA or a non-spliceosomal small nuclear RNA (snRNA); and
a hairpin from a spliceosomal snRNA, a non-spliceosomal snRNA, or a variant of either of these;
wherein the engineered polynucleotide is configured to facilitate exon skipping of the exon in the target RNA.

3. The engineered polynucleotide of claim 1 or 2, wherein the mismatch comprises at least one adenine-guanine (A-G) mismatch, at least one adenine-adenine (A-A) mismatch, or at least one adenine-cytosine (A-C).

4. The engineered polynucleotide of claim 3, wherein the mismatch comprises an A-C mismatch.

5. The engineered polynucleotide of any one of claims 1-4, wherein the Sm or Sm-like protein binding domain or variant thereof and the hairpin are on a 3′ end of the engineered polynucleotide.

6. The engineered polynucleotide of any one of claims 1-5, wherein the targeting sequence is from about 25 bases to about 200 bases in length.

7. The engineered polynucleotide of any one of claims 1-5, wherein the targeting sequence is at least about 30 bases in length.

8. The engineered polynucleotide of any one of claims 1-9, wherein the engineered polynucleotide is operably linked to an RNA polymerase II-type promoter.

9. The engineered polynucleotide of claim 10, wherein the RNA polymerase II-type promoter comprises a U7 promoter.

10. The engineered polynucleotide of any one of claims 1-12, wherein the engineered polynucleotide is operably linked to a U6 promoter.

11. The engineered polynucleotide of any one of claims 1-10, wherein Sm or Sm-like protein binding domain, or variant thereof is a SmOPT sequence.

12. The engineered polynucleotide of claim 11, wherein the SmOPT sequence comprises at least about 80% sequence identity to SEQ ID NO: 41.

13. The engineered polynucleotide of claim 11, wherein the SmOPT sequence comprises the sequence of SEQ ID NO: 41.

14. The engineered polynucleotide of any one of claims 1-13, wherein the hairpin is from a mouse U7 snRNA, a human U7 snRNA, or a human U1 snRNA.

15. The engineered polynucleotide of any one of claims 1-14, wherein the hairpin comprises a sequence that has at least 80%, at least 85%, at least 90%, at least 92%, at least 95%, at least 97%, or at least 99% sequence identity to the hairpin sequence of any one of SEQ ID NO: 42, SEQ ID NO: 43, SEQ ID NO: 45, or SEQ ID NO: 46.

16. The engineered polynucleotide of any one of claim 15, wherein the hairpin comprises the hairpin sequence of any one of SEQ ID NO: 42, SEQ ID NO: 43, SEQ ID NO: 45, or SEQ ID NO: 46.

17. The engineered polynucleotide of any one of claims 1-16, wherein the hairpin comprises the hairpin sequence of SEQ ID NO: 43.

18. The engineered polynucleotide of any one of claims 1-17, further comprising a U7 box terminator at the 3′ end of the engineered polynucleotide.

19. The engineered polynucleotide of any one of claims 1-18, wherein the targeting sequence from 5′ to 3′ comprises the targeting sequence, the Sm or Sm-like protein binding domain or variant thereof, and the hairpin.

20. The engineered polynucleotide of claim 2, wherein the engineered polynucleotide is configured to facilitate editing of a base of a nucleotide of the target RNA by an RNA editing entity.

21. The engineered polynucleotide of any one of claims 1-20, wherein the RNA editing entity comprises an ADAR protein, an APOBEC protein, or both.

22. The engineered polynucleotide of any one of claims 1-20, wherein the RNA editing entity comprises ADAR and wherein the ADAR comprises ADAR1 or ADAR2.

23. The engineered polynucleotide of any one of claims 1-22, wherein the targeting sequence at least partially binds to a target RNA that is implemented in a disease or condition.

24. The engineered polynucleotide of claim 23, wherein the target RNA is selected from the group consisting of RAB7A, ABCA4, SERPINA1, HEXA, LRRK2, SNCA, DMD, APP, Tau, CFTR, ALAS1, ATP7B, HFE, LIPA, PCSK9 start site, or SCNN1A start site, a fragment any of these, and any combination thereof.

25. The engineered polynucleotide of claim 24, wherein the target RNA is SERPINA1, and wherein the SERPINA1 comprises an E342K mutation.

26. The engineered polynucleotide of claim 24, wherein the target RNA is LRRK2, and wherein the LRRK2 comprises an G2019S mutation.

27. The engineered polynucleotide of any one of claims 1-26, wherein the disease or condition comprises Rett syndrome, Huntington's disease, Parkinson's Disease, Alzheimer's disease, a muscular dystrophy, or Tay-Sachs Disease.

28. The engineered polynucleotide of any one of claims 1-27, wherein the targeting sequence is at least partially complementary to a splice signal proximal to an exon within the target RNA.

29. The engineered polynucleotide of claim 28, wherein the targeting sequence is:
(a) at least partially complementary to a branch point upstream of an exon within the target RNA; or
(b) the targeting sequence is at least partially complementary to a donor splice site downstream of an exon within the target RNA.

30. The engineered polynucleotide of any one of claims 1-29, wherein the mismatch is located from about 1 to about 200 bases from either end of the targeting sequence.

31. The engineered polynucleotide of any one of claims 1-29, wherein the mismatch is located at least 45, 46, 47, 48, 49, 50, 51, 52, 53, 54, or 55 bases from either end of the targeting sequence.

32. The engineered polynucleotide of any one of claims 1-31, further comprising a deaminase recruiting domain.

33. The engineered polynucleotide of claim 32, where the deaminase recruiting domain is selected from the group consisting of: GluR2, Alu, a portion of either of these, a variant of either of these, and any combination thereof.

34. The engineered polynucleotide of claim 32 or 33, wherein the deaminase recruiting domain comprises a stem loop.

35. The engineered polynucleotide of claim 34, wherein the stem loop comprises at least about 80% sequence identity to a GluR2 domain.

36. The engineered polynucleotide of any one of claims 1-35, wherein the targeting sequence is configured to at least partially associate with at least a portion of a 3′ or 5′ untranslated region (UTR) of the target RNA.

37. The engineered polynucleotide of any one of claims 1-35, wherein the targeting sequence is configured to at least partially associate with at least a portion of a translation initiation site.

38. The engineered polynucleotide of any one of claims 1-35, wherein the targeting sequence is configured to at least partially associate with at least a portion of an intronic region of the target RNA.

39. The engineered polynucleotide of any one of claims 1-35, wherein the targeting sequence is configured to at least partially associate with at least a portion of an exonic region of the target RNA.

40. The engineered polynucleotide of any one of claims 1-39, wherein the engineered polynucleotide is about 80 nucleotides to about 600 nucleotides.

41. A vector comprising or encoding the engineered polynucleotide of any one of claims 1-40.

42. The vector of claim 41, wherein the vector comprises a liposome, a nanoparticle, or a dendrimer.

43. The vector of claim 41, wherein the vector is a viral vector.

44. The vector of claim 43, wherein the viral vector is an adeno-associated viral (AAV) vector.

45. The vector of claim 44, wherein the AAV vector is an AAV2 vector, AAV5 vector, AAV8 vector, AAV9 vector, or a hybrid of any of these.

46. A pharmaceutical composition in unit dose form comprising the engineered polynucleotide of any of claims 1-40, a polynucleotide encoding engineered polynucleotide of any of claims 1-40, or the vector of any one of claims 41-45; and a pharmaceutically acceptable: excipient, diluent, or carrier.

47. A method of treating or preventing a condition in a subject in need thereof, comprising administering to the subject an effective amount of the engineered polynucleotide of any of claims 1-40, a polynucleotide encoding engineered polynucleotide of any of claims 1-40, or the vector of any one of claims 41-45, or the pharmaceutical composition of claim 46.

48. The method of claim 47, wherein the condition is Duchenne's Muscular Dystrophy (DMD), Rett's syndrome, Charcot-Marie-Tooth disease, Alzheimer's disease, a tauopathy, Parkinson's disease, alpha-1 anti trypsin deficiency, or Stargardt's disease.

49. The method of claim 47, wherein the condition is associated with a mutation in a gene selected from the group consisting of RAB7A, ABCA4, SERPINA1, SERPINA1 E342K, HEXA, LRRK2, SNCA, DMD, APP, Tau, CFTR, ALAS1, ATP7B, ATP7B G1226R, HFE C282Y, LIPA c.894 G>A, PCSK9 start site, or SCNN1A start site, a fragment any of these, and any combination thereof.

50. The method of any one of claims 47-49, wherein the administering is inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration. Delivery can include topical administration (such as a lotion, a cream, an ointment) to an external surface of a surface, such as a skin. In some cases, administration is by parenchymal injection, intra-thecal injection, intra-ventricular injection, intra-cisternal injection, intravenous injection, or intranasal administration or any combination thereof.

51. The method of any one of claims 47-50, wherein the subject is human.
